SEQUENCE LISTING (4 sequences)

SEQ ID NO: 1 - protein, 1054 aa, Streptococcus dysgalactiae
MGTILGTHHDSLISVKAEEKITQVSQTSTSIDDLHYLSENSKKEFKEELSKEKVPEKVKEILSKAQQANKQAQELAEMKVPDKIPMKPLNGPLYGGYFRTWHDKTSDPLEKDKVNSMGELPKEVDLAFVFHDWTKDYSLFWKELATKHVPKLNKQGTRVIRTIPWRFLAGGDNSGIAEDASKYPNTPEGNKALAKAIVDEYVYKYNLDGLDVMIEHDSIPKVNGEASDENLKRSIDVFEEIGKLIGPKGADKSRLFIMDSTYMADKNPLIERGAPYIDLLLVQVYGSQGEQGEFQNDTKSVTKTPEERWQGYSKYIRPEQYMIGFSFYEEKAGSGNLWYDINARKDEDTANGINDDITGTRAERYARWQPKTGGVKGGIFSYAIDRDGVAHQPKQIAEKDKQSVKNNRPLISEITDNIFHSNYSVSKTLKTVMLKDKAYDLIDEKDFPDKALREAVMAQVGTRKGDLERFNGTLRLDNPAIQSLEGLNKFKKLAQLDLIGLSRIIKLDQSVLPANMKPGKDPLETVLETYKKNGKEEPAIIPPVSLTVSGLTGLKELDLSGFDRETLAGIDAATLTSLEKVDISDNKLDLAPKTENRQIFDVMLSTVNNNAGISEQSIKFDNQKPAGNYPQTYGATNLQLPVRQEKIDLQHQLLFGTITNQGTLINSEADYKTYRNQKIAGRNFVDPDYPYNNFKVSHDNYTVKVTDSTLGTTTDKMLATDKEETYKVDFFSPTDKTKAVHTAKVIVGDEKTMMVNLAEGATVIKSENDENAQKVFNGIMEYNPLSFNNKSSIIFEIKDPSLAKYWRLFNDSSKDKKDYIKEAKLEVFTGQLNAEADVKTILEKPDNWVTVSTYSGEEKVFSHSLDNISAKYWRVTVDNKKDQYGYVSLPELQILGYPLPNADTIMKTVTVAKELSQQKDKFPQQLLDESTAKEAVVEASLNSKLFDTGVINTNVEALKNVVDECLAYEKNKETAFKATEDYRAAVNGVKAESVTVEEMAQLKDLIGKAAHLNSKIDAKLADREYDKDLLGLIGELTNITRTVKSFVKHHHHHH

SEQ ID NO: 2 - protein, 998 aa, Streptococcus equi
MVAILAAQHDSLIRVKAEDKLVQTSPSVSAIDALHYLSENSKKEFKEELSKVEKAQPEKLKEIVSKAQQADKQAKTLAEMKVPEKIPMKPLKGPLYGGYFRTWHDKTSDPAEKDKVNSMGELPKEVDLAFVFHDWTKDYSLFWQELATKHVPTLNKQGTRVIRTIPWRFLAGGDHSGIAEDAQKYPNTPEGNKALAKAIVDEYVYKYNLDGLDVMIERDSIPKVNKEESKEGIERSIQVFEEIGKLIGPKGADKSRLFIMDSTYMADKNPLIERGAPYIDLLLVQVYGTQGEKGGFDNANHKAVDTMEERWESYSKYIRPEQYMVGFSFYEEKANSGNLWYDVNVEDDTNPNIGSEIKGTRAERYAKWQPKTGGVKGGIFSYGIDRDGVAHPKKNGPKTPDLDKIVKSDYKVSKALKKVMENDKSYELIDQKDFPDKALREAVIAQVGSRRGNLERFNGTLRLDNPDIKSLEGLNKLKKLAKLELIGLSQITKLDSSVLPENIKPTKDTLVSVLETYKNDDRKEEAKAIPQVALTISGLTGLKELNLAGFDRDSLAGIDAASLTSLEKVDLSSNKLDLAAGTENRQILDTMLATVTKHGGVSEKTFVFDHQKPTGLYPDTYGTKSLQLPVANDTIDLQAKLLFGTVTNQGTLINSEADYKAYQEQEIAGHRFVDSSYDYKAFAVTYKDYKIKVTDSTLGVTDHKDLSTSKEETYKVEFFSPINSTKPVHEAKIVVGEEKTMMVNLAEGATIIGGDADPTNAKKVFDGLLNNDTTTLSTSNKASIIFELKEPGLVKHWRFFNDSKISKADYIKEAKLEAFVGHLEDSSKVKDSLEKSTEWVTVSDYSGEAQEFSQPLNNIGAKYWRITIDNKKSQYGYVSLPELQIIGHRLPEAATVMTTMAAAEELSQQKDKFSQEQLKELEVKVAALKAALDNKMFNADTINASFADVKAYIDKLLADAAGKKTLGKATKEAQPVATDAKEKAESENPKADHHHHHH

SEQ ID NO: 3 - DNA, 3147 bp, Streptococcus dysgalactiae
  source 1..3147: mol_type = genomic DNA
atgggcaccatcctgggtacccaccacgacagcctgatcagcgtgaaggcggaggaaaaaattacccaagttagccaaaccagcaccagcattgacgatctgcactacctgagcgaaaacagcaagaaagagttcaaagaggagctgagcaaggagaaagtgccggaaaaggttaaagagatcctgagcaaagcgcagcaagcgaacaagcaggcgcaagagctggcggaaatgaaggtgccggacaaaattccgatgaagccgctgaacggtccgctgtatggtggctactttcgtacctggcacgacaaaaccagcgatccgctggaaaaggacaaagttaacagcatgggcgaactgccgaaagaggtggatctggcgttcgtttttcacgactggaccaaagattatagcctgttctggaaagagctggcgaccaagcacgtgccgaagctgaacaaacagggtacccgtgttatccgtaccattccgtggcgttttctggcgggtggcgacaacagcggtattgcggaagatgcgagcaagtacccgaacaccccggagggtaacaaagcgctggcgaaggcgattgtggacgaatacgtttataaatacaacctggacggtctggatgtgatgatcgagcacgatagcattccgaaagttaacggcgaagcgagcgacgagaacctgaagcgtagcatcgatgtgttcgaggaaatcggtaaactgattggtccgaaaggcgcggacaagagccgtctgtttattatggacagcacctatatggcggataagaacccgctgatcgaacgtggcgcgccgtatattgacctgctgctggtgcaggtttacggtagccagggcgagcagggtgaattccaaaacgataccaaaagcgttaccaagaccccggaggaacgttggcagggctatagcaaatacatccgtccggagcaatatatgattggtttcagcttttacgaggaaaaggcgggtagcggcaacctgtggtacgacatcaacgcgcgtaaagacgaagataccgcgaacggcatcaacgacgatattaccggtacccgtgcggagcgttatgcgcgttggcagccgaaaaccggtggcgtgaagggtggcatctttagctacgcgattgaccgtgatggtgttgcgcaccagccgaagcaaatcgcggaaaaggacaaacaaagcgtgaaaaacaaccgtccgctgatcagcgagattaccgataacattttccacagcaactatagcgtgagcaagaccctgaaaaccgttatgctgaaggacaaagcgtacgacctgatcgatgaaaaagactttccggataaagcgctgcgtgaggcggtgatggcgcaggttggcacccgtaagggtgacctggaacgtttcaacggcaccctgcgtctggataacccggcgatccagagcctggagggtctgaacaagtttaagaaactggcgcaactggacctgattggcctgagccgtatcattaaactggatcaaagcgtgctgccggcgaacatgaagccgggtaaagacccgctggaaaccgttctggagacctacaagaaaaacggcaaagaggagccggcgatcattccgccggttagcctgaccgttagcggtctgaccggtctgaaagaactggacctgagcggcttcgatcgtgagaccctggcgggtatcgatgcggcgaccctgaccagcctggaaaaggtggacattagcgataacaaactggacctggcgccgaagaccgagaaccgtcagatcttcgatgtgatgctgagcaccgttaacaacaacgcgggtatcagcgagcagagcattaaatttgacaaccaaaagccggcgggcaactatccgcaaacctacggtgcgaccaacctgcagctgccggttcgtcaagaaaaaatcgacctgcagcaccaactgctgttcggcaccatcaccaaccagggtaccctgattaacagcgaggcggattataaaacctaccgtaaccaaaagattgcgggtcgtaacttcgtggacccggattatccgtacaacaactttaaagttagccacgacaactataccgtgaaggttaccgatagcaccctgggcaccaccaccgacaaaatgctggcgaccgataaagaggaaacctacaaggtggacttctttagcccgaccgataagaccaaagcggttcacaccgcgaaagtgatcgttggcgacgaaaagaccatgatggtgaacctggcggagggtgcgaccgttatcaaaagcgagaacgatgaaaacgcgcagaaggttttcaacggtattatggaatataacccgctgagcttcaacaacaagagcagcatcatttttgagatcaaagacccgagcctggcgaagtattggcgtctgttcaacgatagcagcaaagacaagaaagattacatcaaggaagcgaaactggaagtgtttaccggtcagctgaacgcggaagcggacgttaaaaccattctggagaagccggataactgggtgaccgttagcacctatagcggcgaggaaaaggtgtttagccacagcctggacaacatcagcgcgaaatactggcgtgtgaccgttgacaacaagaaagatcagtatggctacgttagcctgccggagctgcaaatcctgggttacccgctgccgaacgcggataccattatgaaaaccgtgaccgttgcgaaggaactgagccagcaaaaggacaaattcccgcagcaactgctggatgagagcaccgcgaaggaagcggtggttgaggcgagcctgaacagcaaactgtttgacaccggtgtgatcaacaccaacgttgaagcgctgaagaacgtggttgatgagtgcctggcgtatgaaaagaacaaagagaccgcgttcaaggcgaccgaagactaccgtgcggcggtgaacggtgttaaagcggagagcgtgaccgttgaggaaatggcgcagctgaaagatctgatcggcaaggcggcgcacctgaacagcaaaattgacgcgaagctggcggatcgtgaatacgacaaagatctgctgggcctgatcggcgagctgaccaacattacccgtaccgtgaaaagctttgttaagtga

SEQ ID NO: 4 - DNA, 2979 bp, Streptococcus equi
  source 1..2979: mol_type = genomic DNA
atggttgcgatcctggcggcgcaacacgatagcctgattcgtgtgaaggcggaggacaaactggtgcagaccagcccgagcgttagcgcgattgatgcgctgcactacctgagcgaaaacagcaagaaagaattcaaagaggaactgagcaaggttgaaaaagcgcaaccggagaagctgaaagaaatcgtgagcaaggcgcagcaagcggacaagcaggcgaaaaccctggcggagatgaaggttccggaaaaaattccgatgaagccgctgaaaggcccgctgtatggtggctactttcgtacctggcacgataaaaccagcgacccggcggagaaggataaagtgaacagcatgggcgagctgccgaaagaagtggacctggcgttcgtttttcacgattggaccaaggactatagcctgttctggcaagaactggcgaccaaacacgttccgaccctgaacaagcagggcacccgtgtgatccgtaccattccgtggcgttttctggcgggtggcgatcacagcggtatcgcggaggacgcgcagaaatacccgaacaccccggaaggcaacaaggcgctggcgaaagcgattgtggatgagtacgtttataagtacaacctggacggtctggatgttatgatcgaacgtgacagcattccgaaggtgaacaaagaggaaagcaaagagggtatcgaacgtagcattcaggttttcgaggaaatcggcaagctgattggtccgaagggcgcggataaaagccgtctgtttatcatggatagcacctatatggcggacaagaacccgctgatcgagcgtggtgcgccgtatattgacctgctgctggtgcaggtttacggtacccagggcgaaaaaggtggcttcgataacgcgaaccacaaggcggttgacaccatggaggaacgttgggagagctatagcaaatacatccgtccggaacaatatatggtgggtttcagcttttacgaggaaaaggcgaacagcggcaacctgtggtacgacgtgaacgttgaggacgataccaacccgaacatcggtagcgagattaaaggcacccgtgcggaacgttatgcgaagtggcagccgaaaaccggtggcgttaagggtggcatctttagctacggtattgaccgtgatggcgtggcgcacccgaagaaaaacggtccgaaaaccccggacctggataagatcgtgaaaagcgattataaagttagcaaagcgctgaagaaagttatggagaacgacaagagctacgaactgatcgaccaaaaggatttcccggacaaagcgctgcgtgaggcggtgattgcgcaggttggtagccgtcgtggcaacctggaacgttttaacggtaccctgcgtctggataacccggacatcaaaagcctggagggcctgaacaaactgaagaaactggcgaagctggaactgatcggtctgagccaaattaccaagctggatagcagcgttctgccggagaacattaagccgaccaaagacaccctggtgagcgttctggaaacctacaaaaacgacgatcgtaaggaagaggcgaaagcgatcccgcaggtggcgctgaccattagcggtctgaccggcctgaaggagctgaacctggcgggtttcgaccgtgatagcctggcgggtattgatgcggcgagcctgaccagcctggagaaagtggatctgagcagcaacaagctggacctggcggcgggtaccgaaaaccgtcaaattctggacaccatgctggcgaccgtgaccaaacacggtggcgttagcgagaagaccttcgtgtttgatcaccagaaaccgaccggtctgtatccggacacctacggcaccaagagcctgcagctgccggttgcgaacgataccatcgacctgcaagcgaaactgctgttcggtaccgtgaccaaccagggcaccctgatcaacagcgaagcggactataaggcgtaccaggagcaagaaattgcgggccaccgtttcgttgatagcagctatgactacaaagcgtttgcggtgacctacaaggattacaagatcaaggttaccgacagcaccctgggtgtgaccgatcacaaagacctgagcaccagcaaagaggagacctataaagttgagttctttagcccgatcaacagcaccaaaccggtgcacgaagcgaagattgtggttggcgaggaaaagaccatgatggttaacctggcggagggcgcgaccatcattggtggcgacgcggacccgaccaacgcgaagaaagtgttcgatggcctgctgaacaacgacaccaccaccctgagcaccagcaacaaagcgagcatcatttttgagctgaaagaaccgggtctggtgaagcactggcgtttctttaacgatagcaagatcagcaaagcggactacattaaagaggcgaaactggaagcgttcgttggtcacctggaagatagcagcaaggtgaaagacagcctggagaaaagcaccgaatgggtgaccgttagcgattatagcggcgaggcgcaggaatttagccaaccgctgaacaacatcggcgcgaagtactggcgtatcaccattgacaacaagaaaagccagtatggttacgttagcctgccggagctgcaaatcattggtcatcgtctgccggaagcggcgaccgtgatgaccaccatggcggcggcggaggaactgagccagcaaaaggataaattcagccaggagcaactgaaggagctggaagtgaaagttgcggcgctgaaggcggcgctggataacaaaatgttcaacgcggacaccatcaacgcgagctttgcggacgttaaagcgtacattgataagctgctggcggacgcggcgggtaagaaaaccctgggcaaagcgaccaaagaggcgcagccggtggcgaccgatgcgaaggaaaaagcggagagcgaaaacccgaaggcggactaa

(19) United States
(12) Patent Application Publication
(10) Pub. No.: US 2026/0224724 A1
(43) Pub. Date: Aug. 6, 2026

(54) GLYCOSYNTHASE VARIANTS FOR ANTIBODY-DRUG CONJUGATE ENGINEERING

(71) Applicant: OBI Pharma, Inc., Taipei City (TW)

(72) Inventors: Ming-Tain LAI, Taipei City (TW); Jun-Hong CHOU, Taipei City (TW); Yin-Cheng HSIEH, Taipei City (TW); Teng-Yi HUANG, Taipei City (TW); Chen-Ping HUANG, Taipei City (TW); Ka-Shu FUNG, Taipei City (TW); Chi-Sheng HSIA, Taipei City (TW)

(21) Appl. No.: 19/127,895

(22) PCT Filed: Sep. 22, 2023
(86) PCT No.: PCT/US2023/074849

§ 371 Date: May 7, 2025

Related U.S. Application Data
(60) Provisional application No. 63/382,951, filed on Nov. 9, 2022.

(51) Int. Cl. A61K 47/68; A61K 47/60; A61P 35/00; C07K 16/24; C07K 16/28; C07K 16/30; C07K 16/32; C12N 9/24; C12P 21/00

(52) U.S. Cl. CPC A61K 47/68037; A61K 47/60; A61K 47/6845; A61K 47/6849; A61K 47/6855; A61K 47/6889; A61P 35/00; C07K 16/241; C07K 16/2818; C07K 16/2827; C07K 16/2863; C07K 16/2887; C07K 16/30; C07K 16/32; C12N 9/2402; C12P 21/005; C07K 2319/30; C07K 2319/91; C12Y 302/01096

(57) ABSTRACT

The present disclosure relates to novel glycosynthase enzymes for antibody-drug conjugates (ADCs) engineering. The enzyme variants, termed EndoSd-D232M and EndoSz-D234M, contain the glycan conjugation and/or modification activity at the conserved N297 glycosylation site of Fc region of an exemplary antibody. It has been demonstrated that the glycosynthase activities of EndoSd-D232M and EndoSz-D234M can be applied to various mAbs targeting different receptors, including, but not limited to, Globo H, SSEA-4, SSEA-3 series of receptors (OBI-888; Globo H ganglioside), Herceptin (Her 2 receptor), Perjeta (Her 2 receptor) and Vectibix (EGFR receptor). It has been found that both mAb-GlcNAc and mAb-GlucNAc(F) were suitable substrates for both EndoSd-D232M and EndoSz-D234M.

### CROSS-REFERENCE TO RELATED APPLICATIONS

This application is a 371 National Phase application of International Patent Application No. PCT/US23/74849, filed Sep. 22, 2023, which claims the priority to and benefit of U.S. Provisional Patent Applications No. 63/382,951, filed Nov. 9, 2022. The entirety of the aforementioned applications are incorporated herein by reference.

### SEQUENCE LISTING

The instant application contains a Sequence Listing which has been submitted electronically in ASCII XML format under Rule ST.26 and is hereby incorporated by reference in its entirety. Said ASCII copy, created on Apr. 25, 2025, is named G3004-01800NP_20250425_SeqListing.xml and is 12,780 bytes in size.

### FIELD OF THE INVENTION

The present disclosure relates to glycosylated antibody conjugates and methods for preparing the same.

### BACKGROUND OF THE INVENTION

Numerous surface carbohydrates are expressed in malignant tumor cells. For example, the carbohydrate antigen Globo H (Fucα1→2 Galβ1→3 GalNAcβ1→3 Galα1→4 Galβ1→4 Glc) was first isolated as a ceramide-linked Glycolipid and identified in 1984 from breast cancer MCF-7 cells. (Bremer E G, et al. (1984) J Biol Chem 259:14773-14777). Previous studies have also shown that Globo H and stage-specific embryonic antigen 3 (Galα1→3GalNAcβ1→3Galα1→4Galβ1→4Glcβ1) (SSEA-3, also called Gb5) were observed on breast cancer cells and breast cancer stem cells (W W Chang et al. (2008) Proc Natl Acad Sci USA, 105(33): 11667-11672). In addition, SSEA-4 (stage-specific embryonic antigen-4) (Neu5Acβ2→3Galβ1→3GalNAcβ1→3Galα1→4Galβ1→4Glcβ1) has been commonly used as a cell surface marker for pluripotent human embryonic stem cells and has been used to isolate mesenchymal stem cells and enrich neural progenitor cells (Kannagi R et al. (1983) EMBO J, 2:2355-2361). These findings suggest that Globo series antigens (Globo H, SSEA-3, and SSEA-4) can be unique targets for cancer therapies and may be used as direct therapeutic agents to target cancer cells effectively.

Program death 1 (PD-1) is an inhibitory receptor expressed on T cells, B cells, or monocytes (Ishida et al. (1992) EMBO J. 11: 3887-2895; Agata et al. (1996) Int. Immunol. 8: 765-772). PD-L1 and PD-L2 are ligands for PD-1 that have been identified to downregulate T cell activation and cytokine secretion upon binding to PD-1 (Freeman et al. (2000) J Exp Med 192:1027-34; Latchman et al. (2001) Nat Immunol 2:261-8). Engagement of PD-1 with PD-L1 or PD-L2 leads to down-regulation of immune responses. Hence, blocking of the PD-1 or PD-L1 antigen pathway has been proposed to attenuate central and peripheral immune responses against cancer. Targeting PD-1 and PD-L1 antigens pathway have shown to have clinical efficacy in more than 15 cancer types including melanoma, non-small cell lung cancer (NSCLC), renal cell carcinoma (RCC), bladder carcinoma, and Hodgkin's lymphoma (Sharma et al. (2015) Science 348(6230):56-61). However, many patients still fail to respond to PD-1 or PD-L1 antigen therapy. In some cases, patients showed initial responses but acquired resistance over time. Therefore, there is an urgent need to identify mechanisms of resistance for PD-1 or PD-L1 antigen combination therapy.

Therapeutic monoclonal antibodies (mAbs) have been developed for the treatment of many diseases, such as cancer, autoimmune, and infectious (Adams, G. P., and Weiner, L. M. (2005) Nat. Biotechnol. 23: 1147-1157; Aggarwal, S. R. (2012) Nat. Biotechnol. 30: 1191-1197; Aggarwal, S. R. (2014) Nat. Biotechnol. 32:323-330). For cancer therapy, several mAbs have already been approved. Those mAbs recognize particular biomarkers on the tumor cell surface and enhance cell apoptosis by different mechanisms, such as turning on antibody-dependent cellular cytotoxicity (ADCC) and complement dependent cytotoxicity (CDC) or blocking signal pathway. Her 2 receptor is the most famous biomarker overexpressed in breast cancer, which led to the development of two related mAbs, Herceptin (trastuzumab) and Perjeta (pertuzumab), by Roche. EGFR receptor is also a well-known cancer target for mAb development. For example, Vectibix (panitumumab) and Erbitux (cetuximab) have been developed by Amgen and Merck Company, respectively, targeting metastatic colorectal cancer therapy. In addition, Rituxan (rituximab, Roche) and Arzerra (ofatumumab, GSK) designed to recognize CD20 receptors are commonly used to treat non-Hodgkin's B-cell lymphomas and chronic lymphocytic leukemia cancer. Most recently, OBI Pharma. Inc. is developing OBI-888 and OBI-898 antibodies based on the ganglioside biomarkers Globo H and SSEA-4, which are found in breast, lung, ovary, stomach, and small-cell lug (Hakomori, S. I. (2008) Biochim. Biophys. Acta. 1780: 325-346; Hakomori, S. and Zhang, Y. (1997) Chem. Biol. 4:97-104; Zhang, S. et al. (1997) Int. J. Cancer 73: 42-49; Zhang, S. et al. (1997) Int. J. Cancer 73: 50-56.) but not detectable in normal cells. Erbitux, Rituxan, Arzerra, OBI-888, and OBI-898 kill cancer cells through cytotoxicity in terms of ADCC and CDC. Moreover, several mAbs have been developed to block the function of protein-protein interaction. For example, Humira (adalimumab, AbbVie) blocks the TNF-α receptor mediate signal pathway for autoimmune disease, rheumatoid arthritis; Keytruda (pembrolizumab, Merck) blocks PD-1 receptor to destroy the protective mechanism of cancer cells that treats metastatic melanoma. Compared with small molecule drugs, mAbs are more specific to the target cells and have relatively fewer side-effect to the patients. These two important features have become powerful tools against various diseases.

Monoclonal antibodies (mAbs) have a molecular weight of ~150 kDa composed of two heavy chains (~50 kDa) and two light chains (~25 kDa), which form three domains separated by a flexible hinge region. Two Fab domains contain variable complementarity-determining regions (CDR) for identifying antigens. One Fc domain is a constant region with N-glycan for mediation of ADCC and CDC cytotoxicity (Jefferis, R. (2009) Nat. Rev. Drug Discoy. 8:226-234). The amino acid N297 in the Fc domain is a conserved N-glycosylation site that connects with heterogeneous glycan types, such as biantennary (M3, G0F, G1F, G2F, G0, G1, and G2 complex type) and triantennary (high-mannose and hybrid types), while expressed in various cell systems. The X-ray structure analysis results indicated that the core fucose of Fc glycan obstructed the particular carbohydrate-carbohydrate interactions between Fc and FcγRIIIa and decreased the binding constant for approximately hundred folds (Ferrara, C. et al. (2011) Proc. Natl. Acad. Sci. USA 108: 12669-12674) and reduced cell killing efficiency. The most common cell system used in the biopharmaceutical industry is CHO cell. CHO cells generally produce the mAbs that contain glycan compositions predominately in the form of G0F, G1F and G2F. These glycan forms limit the ADCC activity of the mAbs due to the reduced ADCC binding efficiency caused by fucose. Although modified CHO cell systems have been available by FUT8 (α-1,6-fucosyltransferase 8) gene knock-out (Yamane-Ohnuki, N. and Satoh, M. (2009) MAbs, 1: 230-236; Yamane-Ohnuki, N. et al. (2004) Biotechnol. Bioeng. 87: 614-622) or up-regulation of bisecting GlcNAc (N-acetylglycosamine) transferase GnT-III (Umana, P. et al. (1999) Nat. Biotechnol. 17: 176-180) to reduce the glycan complicity of mAbs, there is still a great interest to find a better and more general methodology to obtain the desired N-glycan on mAbs. In addition, it has also been reported that removing the Fc glycan will result in loss of the ADCC activity (Kurogochi, M. et al. (2015) PLoS One 10: e0132848). All these data suggest that mAbs cytotoxicity can be effectively controlled by the N-glycan type attached on the Fc region.

Enzymatic modification of the Fc region is a solution to establish homogeneous mAbs. Lai-Xi Wang and coworkers have tried the chemoenzymatic remodeling by removing the glycan mixture and conjugating homogeneous glycans (Huang, W. et al. (2012) J. Am. Chem. Soc. 134: 12308-12318). Several endo-β-N-acetylglycosaminidases (ENGases) have also been reported to remove glycan mixture on mAbs. For instance, EndoD (Tai, T. et al. (1975) J. Biol. Chem. 250: 8569-8575), EndoH (Tarentino, A. L. et al. (1974) J. Biol. Chem. 249: 818-824), EndoLL (Kurogochi, M. et al. (2015) PLoS One 10: e0132848) and EndoM (Kadowaki, S. et al. (1990) Agric. Biol. Chem. 54: 97-106) are able to hydrolyze the glycan with high mannose or terminal mannose types. EndoS (Collin M and Olsen A. (2001) EMBO J. 20: 3046-3055) and EndoSd (Shadnezhad, A. et al. (2016) Future Microbiol 11: 721-736.) have the ability to hydrolyze non-fucosylated and fucosylated N-glycans on the Fc domain, but not high-mannose types. Up-to-date, no single endoglycosidase could completely hydrolyze all glycan types on mAbs. The EndoS crystal structure was recently solved, which revealed five functional domains (Trastoy, B. et al. (2014) Proc. Natl. Acad. Sci. USA 111: 6714-6719), in which the endoglycosidase domain is highly conserved with rigid β-barrel structure that is suitable for site mutation studies. On the other hand, glycosynthases for antibody Fc were also reported. EndoD-N322Q (Fan, S. Q. et al. (2012) J. Biol. Chem. 287: 11272-11281) and EndoM-N175Q (Umekawa, M., Li, C. et al. (2010) J. Biol. Chem. 285: 511-521) only transferred short chain complex-type N-glycan to Fc. Endo-F3-D165Q (Giddens, J. P. et al. (2016) J. Biol. Chem. 291: 9356-9370) only transferred glycan to the fucosylated Fc domain. EndoS-D233Q (Huang, W. et al. (2012) J. Am. Chem. Soc. 134: 12308-12318) enables conjugation of various bi-antennary complex types, whereas EndoS2-D184M (Li., T., Tong, et al. (2016) J. Biol. Chem. 291: 16508-16518) has wild substrates including complex, high-mannose and hybrid types.

The use of Antibody-Drug Conjugates (ADCs) for the local delivery of cytotoxic or cytostatic drugs to kill or inhibit tumor cells in the treatment of cancer (Syrigos and Epenetos (1999) Anticancer Research 19:605-614; Niculescu-Duvaz and Springer (1997) Adv. Drg. Del. Rev. 26:151-172; U.S. Pat. No. 4,975,278) allows targeted delivery of the drug moiety to tumors, and intracellular accumulation therein, while systemic administration of the unconjugated cytotoxic or cytostatic drugs may result in unacceptable levels of toxicity to normal cells as well as to the tumor cells sought to be eliminated (Baldwin et al., 1986, Lancet pp. (Mar. 15, 1986):603-05; Thorpe, 1985, “Antibody Carriers Of Cytotoxic Agents In Cancer Therapy: A Review,” in Monoclonal Antibodies '84: Biological And Clinical Applications, A. Pinchera et al. (ed.s), pp. 475-506). Both polyclonal antibodies and monoclonal antibodies have been reported as useful in these strategies (Rowland et al., 1986, Cancer Immunol. Immunother. 21:183-87). Drugs used in these methods include daunomycin, doxorubicin, methotrexate, and vindesine (Rowland et al., 1986, supra). Some cytotoxic drugs tend to be inactive or less active when conjugated with large antibodies or protein receptor ligands.

### SUMMARY OF INVENTION

In one aspect, the present disclosure provides a method for preparing an engineered bioconjugate, comprising contacting a biomolecule with a glycosynthase and a modified glycan thereby obtaining a first engineered bioconjugate, wherein the biomolecule further comprises a N-linked initial glycan; wherein the glycosynthase comprises SEQ ID NO. 1 or SEQ ID NO. 2, and the glycosynthase comprises a mutation located within residues 176-186, residues 225-237, residues 273-289 in the sequence of SEQ ID NO. 1 or within residues 178-188, residues 227-239, residues 275-291 in the sequence of SEQ ID NO. 2; wherein the modified glycan comprises a substrate moiety and a first reactive moiety; wherein the substrate moiety is configured to react with the glycosynthase; and wherein the biomolecule comprises an antibody or antigen binding fragment thereof, a protein, or a peptide.

In another aspect, the present disclosure provides an engineered bioconjugate, comprising: a biomolecule and a modified glycan, coupled with the biomolecule; wherein the modified glycan comprises: (i) a first polyethylene glycol (PEG) moiety, and (ii) a first reactive moiety or a resultant moiety thereof from a biorthogonal reaction; wherein the resultant moiety comprises a triazole moiety, a DBCO-derived moiety, or a maleimide-derived moiety; and wherein the biomolecule comprises an antibody or antigen binding fragment thereof, a protein, or a peptide.

In another aspect, the present disclosure provides a pharmaceutical composition, comprising the plurality of engineered bioconjugates of the present disclosure and a pharmaceutically acceptable carrier.

In another aspect, the present disclosure provides a method for treating cancer, comprising administering to a patient in need thereof an effective amount of the pharmaceutical composition of the present disclosure.

In another aspect, the present disclosure provides a therapeutic conjugate comprising a formula of:

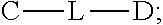

wherein C is a reactive moiety, configured to react in a biorthogonal reaction; wherein L is a linker unit comprising a hydrophilic moiety, a cleavable moiety, and a spacer; and wherein D is a therapeutic agent.

### BRIEF DESCRIPTION OF THE DRAWINGS

FIG. 1. Summarizes aspects of the overall process of homogeneous platform. (A) The mAbs were heterogeneous with glycan mixtures which were removed by wild type EndoSz and α-fucosidase to generate mAb-GlcNAc. Then the EnodSd-D232M and EnodSz-D234M were used to conjugate glycan-oxazoline and produce homogeneous mAbs. (B) In the glycan cleavage step, only EndoSz enzyme is used to generate mAbs-GlcNAc-F and the product will be homogeneous-mAb-glycan-F after conjugation. (C) The exemplary picture of biantennary glycans.

FIG. 2. LC/MS/MS results of Herceptin-GlcNAc glycopeptide. The Herceptin wild type was mixed with EndoSz wild type and α-fucosidase to remove the glycans on the Fc region. The result showed all of the glycans were removed and generated >99% Herceptin-GlcNAc.

FIG. 3. The exemplary detection methods for homogeneous platform (demonstrated by EndoSz-D234M). (A) The HPLC analysis method. Original Herceptin (green) had retention time of 12.5 minute, and Herceptin-GlcNAc (magenta) shifted to 11.4 minutes. In the transglycosylation of NSCT-oxa, the Herceptin-G2S2 (blue) could be clearly distinguished by 1N-G2S2 (hemi-glycosylated) and 2N-G2S2 (fully glycosylated) with retention time 12.6 and 13.9 minutes, respectively. In the transglycosylation process, the peaks sequentially shifted from Herceptin-GlcNAc to Herceptin-1N-G2S2 and Herceptin-2N-G2S2, which allowed us to monitor the process. (B) The SDS-PAGE results for comparison. Hemi-glycosylated and fully glycosylated Herceptin-G2S2 could not be easily identified.

FIG. 4. The multiple sequence alignment of exemplary EndoS2, EndoS, EndoSz and EndoSd. The loops surrounding the active site are labeled as light blue.

FIG. 5. The time dependent transglycosylation results (A) The transglycosylation of NSCT-oxa and Herceptin-GlcNAc with molar ratio 20:1 by EndoSz-D234M. (B) EndoSd-D232M used a higher molar ratio of 150:1 (NSCT-oxa:Herceptin-GlcNAc). The reaction was started with 100% Herceptin-GlcNAc. According to the efficiency, the percentage of Herceptin-1N-G2S2 and Herceptin-2N-G2S2 were formed. Tansglycosylation efficiency of >90% Herceptin-2N-G2S2 could be reached in both enzymes.

FIG. 6. The relative transglycosylation activities in different exemplary mutants. (A) EndoSz (B) EndoSd.

FIG. 7. Demonstration of homogeneous mAbs efficacy: The ADCC assay results. (A) The ADCC result of Herceptin and Herceptin-G2S2. The data showed Herceptin had higher ADCC activity. The EC₅₀ of Herceptin and Herceptin-G2S2 was 15.29 (μg/mL) and 5.10 (μg/mL), respectively. (B) The summary of the transglycosylation efficiency and ADCC in various mAbs.

FIG. 8. Overall architecture of apo EndoSz. (A) The glycosidase (red), leucine-rich repeat (yellow), hybrid lg (light blue), carbohydrate-binding motif (orange), and C-3HB (purple) domains are shown as cartoons. (B) Top and side views of the electrostatic surfaces are shown. (C) The views of the conservation surface of EndoSz.

FIG. 9. Structure of the bound complex biantennary glycan on EndoSz-D234M. (A) The 2Fo-Fc electron density map contoured to 1.00 is shown as blue meshes. (B) Structure of the bound N-glycan (gray sticks) in the β-barrel flanked with loops which are annotated. The EndoSz-D234M is shown as transparent gray. (C) Cartoon representation of the substrate and of EndoSz-D234M, the complex-type N-linked glycan. The product is shown in the dashed rectangle. (D) The variable loops (green) in the conservation plot are labeled. The loop1 (red ribbon), loop2 (orange ribbon), loop3 (yellow ribbon), loop4 (green ribbon), loop5 (blue ribbon), loop6 (cyan ribbon), loop7 (purple ribbon) and loop8 (wheat ribbon) surrounding the active site are labeled. The α (1-6) antenna of the CT N-glycan is labeled.

FIG. 10. The sugar substrate selectivity is dominated by the loop 4 between EndoSz and EndoS2. (A) The superimposed structures of EndoSz/CT-N-glycan and EndoS2/HM-N-glycan. The tilted Helix 3 (labeled as H3) in EndoSz structurally results in the large loop 4 variation to hinder the binding of HM-N-glycan is labeled. The loop 4 is indicated by the red arrow. The EndoSz/CT-N-glycan (green) and EndoS2/HM-N-glycan (blue) are colored respectively. (B) The superimposed structures of EndoS2/HM-N-glycan and unbound EndoS2. The EndoS2/HM-N-glycan (blue) and unbound EndoS2 (wheat) are colored respectively.

FIG. 11. Movement of the loop2 shapes the two binding grooves after the N-glycan binding. (A) Stereo view of the superimposed structures of apo- and holo-EndoSzD234M. The apo-EndoSz-D234M (blue) and holo-EndoSz-D234M (gold) are shown as cartoons. The bound N-glycan in the holo-EndoSz-D234M is shown as balls and sticks. The key residues W154(sticks) and interacted Man (−2) and NAG (−8) are labeled. (B) Electrostatic surfaces of the unbound (left) and the N-glycan bound (right) EndoSz-D234M structures. W154 is indicated in the black circle. (C) Sequence alignment and the conservation plot of the loop2 of EndoSz-D234M.

FIG. 12. The 2D diagram of EndoSz-D234M-sugar interactions. (A) The conformation A of GlcNAc (−1). (B) The conformation B of GlcNAc. The bound CT N-glycan is shown as purple sticks. The interacted residues of EndoSz with hydrogen bonds (orange) and hydrophobic interactions (red) are shown as orange sticks and red eyes respectively. The hydrogen-bound residues (blue) and the residues with the hydrophobic contacts (black) are labeled.

FIG. 13. The SDS-PAGE and CE-SDS results of mAb-(NSCT-di-N3)₂ production. (A) SDS-PAGE of R4702 (Anti-TROP2 mAb), (B) SDS-PAGE of TX05 (Anti-H,ER2 mAb) and (C) CE-SDS result analysis.

FIG. 14. The result of MW analysis by intact MS.

FIG. 15. The LC and HC MS spectrum of R4702-MCCA-ADC (ADC-1) and R4702-DBCO-ADC (ADC-2) incubated with HSA for (A) Day 0 and 6 of ADC-1; (B) Day 0 and 6 of ADC-2; (C) DAR change profiles. LC0, LC1, HC0, HC1, HC2, and HC3 devote the spectrum of the light chain and heavy chain with 0, 1, 2, 3 payloads, respectively.

FIG. 16. The Payload release over time of R4702-MCCA-ADC (ADC-1) and R4702-DBCO-ADC (ADC-2) in human plasma.

FIG. 17. Human tumor cell line cytotoxicity assay. Serial dilutions of ADCs were used to evaluate the in vitro cytotoxicity efficacy in several human tumor cell lines. Tumor cells were cultured with ADCs for 6 days and viable cells were analyzed by CellTiter-Glo®. The IC₅₀ of each ADC was calculated by Prism. (A) NCI-H1975-C797S (human lung cancer) and DU-145 (human prostate cancer) tumor cells were used to evaluate the efficacy of R4702-MCCA-ADC (ADC-1) and R4702-DBCO-ADC (ADC-2). (B) NCI-N87 (human gastric cancer) and Capan-1 (human pancreatic cancer) tumor cells were used to evaluate the efficacy of TX05-MCCA-ADC (ADC-3) and TX05-DBCO-ADC (ADC-4).

FIG. 18. Evaluation of in vivo efficacy in NCI-H1975-C797S human lung cancer xenograft mouse model. NCI-H1975-C797S cells were cultivated and implanted subcutaneously into the right flank of BALB/c nude mice. Tumor-bearing mice were treated at 10 or 3 mg/kg as a single dose when the average tumor volume reached 150-200 mm³. Tumor volume (A) and body weight (B) were monitored twice weekly until Day 22. The tumor growth inhibition (TGI) was calculated by the following formula: TGI (%)=[1−(Ti−T1)/(Ci−C1)]×100%. Ti and Ci indicate the mean tumor volume in the treatment groups and vehicle group at the end of the study (Day 22), while T1 and C1 indicate the mean tumor volumes in the treatment group and vehicle group at the beginning of test item administration.

### ABBREVIATIONS

ACN: acetonitrile; ADCC: antibody-dependent cellular cytotoxicity; DBCO: Dibenzocyclooctyne; CDC: complement dependent cytotoxicity; CDR: complementarity-determining region; FA: Formic acid; FUT8: α-1,6-fucosyltransferase 8; GlcNAc: N-acetylglycosamine; HFIP: 1,1,1,3,3,3-Hexafluoro-2-propanol; IPTG: isopropyl-β-D-thiogalactopyranoside; TFA: trifluoroacetic acid; DMSO: Dimethyl sulfoxide; NaOAc: Sodium acetate; NaOH: Sodium hydroxide; HIC: hydrophilic interaction chromatography; ADC: Antibody drug conjugate; mAb: Monoclonal antibody; ISTD: internal standard; HSA: human serum albumin; PBS: phosphate buffered saline; HC: heavy chain; LC: light chain; DAR: Drug antibody ratio; HRMS: High Resolution Mass Spectrometer; DL: Drug linker; NSCT: Sialylated complex type N-glycan; ADC-1: R4702-MCCA-ADC; ADC-2: R4702-DBCO-ADC; ADC-3: TX05-MCCA-ADC; ADC-4: TX05-MCCA-ADC.

### DETAILED DESCRIPTION OF THE INVENTION

Antibody-Drug Conjugates (ADCs) are a promising approach to deliver a therapeutic agent or an imaging agent to a target cell with reliable specificity and efficiency. The conjugation of a payload (e.g., a therapeutic agent or an imaging agent) on an antibody usually takes place on the lysine or cysteine residues of the antibody. The drug-to-antibody ratio (DAR) of an ADC will therefore be decided according to the amount of the lysine or cystine residues on the antibody. However, practically, not every antibody in a conjugation reaction will be conjugated identically, resulting in isomers of the ADC having a variety of DARs and conjugation sites. On top of that, conjugation via lysine or cysteine residues entails using a reducing reagent, which is another concern as the reducing reagent might affect the conformation of the antibody thereby affecting the efficacy of the ADC. With the aforesaid technical problem in mind, the present disclosure is directed to an engineered antibody conjugate configured to conjugate a payload via a glycan on the antibody. The preparation of the engineered antibody conjugate of the present disclosure involves glycosylation (e.g., N-glycosylation) that couples a proper glycan with the antibody.

N-glycosylation is one of the most complex post-translational modifications that often result in a remarkable heterogeneity of glycan structures including high mannose, hybrid and complex types, depending on the recombinant expression system. Commercially available therapeutic antibodies typically exist as mixtures of glycoforms that are not optimal for their respective therapeutic activities. Recently, glycoengineering has gathered attention to control Fc glycosylation for improving efficacy. Endoglycosidases are a family of at least 18 glycoside hydrolase (GH) from the Streptococcus pyogenes and have recently become the point of attention for glycoengineering of therapeutic antibodies. These enzymes can catalyze the hydrolysis of the β-1,4 linkage between the two N-acetylglucosamines (GlcNAcs) in the core of the N-linked glycan of human IgG. Additionally, the enzymes remove complex type glycans at IgG Fc domain.

In order to conjugate with a payload, the glycan coupled with the antibody needs to be modified to provide functional groups for the conjugation. Functional groups of a modified glycan can affect the glycosylation efficiency of an endoglycosidase. Consequently, the glycosylated antibodies might have a poor homogeneity. Although there are endoglycosidases known in the field, their capabilities of glycosylating a modified glycan cannot be foreseen from their capabilities of glycosylating a non-modified glycan. Therefore, it is critical to identify a capable endoglycosidase and a suitable modified glycan for the purpose of solving the aforesaid technical problems.

One aspect of the present disclosure is directed to methods for preparing an engineered bioconjugate. The bioconjugate can be a glycosylated antibody or antigen-binding fragment thereof, a glycoprotein, or a glycopeptide. Another aspect of the present disclosure is directed to an engineered bioconjugate or a plurality of the engineered bioconjugates. Yet another aspect of the present disclosure is directed to pharmaceutical compositions comprising the engineered bioconjugates. Yet another aspect of the present disclosure is directed methods for treating cancer using the pharmaceutical composition. Yet another aspect of the present disclosure is directed to a therapeutic conjugate.

Embodiments of the present disclosure relate to selected variants of glycosynthase that show remarkable transglycosylation activities to transfer a broad range of N-glycans of high mannose, hybrid or complex types, from activated oligosaccharide oxazolines to fucosylated or non-fucosylated GlcNAc-peptides, proteins or IgGs with little or negligible product hydrolysis. The novel Glycosynthase enzymes acted with surprisingly high efficiency to provide homogeneously glycosylated glycopeptides, glycoproteins and therapeutic antibodies and Fc fragments thereof, having various defined glycoforms. Still, further, embodiments of the present disclosure may provide glycoengineered antibodies with enhancement of their effector functions, such as FcγIIIA bindings and antibody dependent cell mediated cytotoxicity (ADCC), etc., as well as pharmacological properties. Embodiments of the present disclosure also allow for rapid investigation of effects of diverse Fc glycosylations of therapeutic antibodies on their effector functions.

In accordance with embodiments of the present disclosure, a novel glycosynthase enzyme comprises a sequence selected from the sequences of SEQ ID NOs. 1-2. These mutants show unexpectedly improved transglycosylation activities and reduced hydrolyzing activities. Therefore, they can catalyze efficient transfer of activated oligosaccharide donors to core GlcNAc-acceptors, which may be fucosylated or non-fucosylated.

In accordance with certain embodiments, a glycosynthase enzyme may have a sequence identity of at least about 80% (e.g., 80%, 85%, 90%, 95%, or 98% (or a value ranging between any of the two numbers listed herein) to a sequence in SEQ ID Nos. 1-2 and have the desired transglycosylation activity, or fragment thereof having the transglycosylation activity. In some embodiments, the glycosynthase is as described in the U.S. Pat. No. 11,203,645, filed on Jun. 27, 2019, which is hereby incorporated by reference.

### Definitions

It must be noted that as used herein and in the appended claims, the singular forms “a”, “an”, and “the” include plural reference unless the context clearly dictates otherwise. The term “ones” means more than one. As used herein, the term “plurality” can be 2, 3, 4, 5, 6, 7, 8, 9, 10, or more.

As used herein, the term “comprise,” “include,” or “have” used herein is intended to describe the presence of state features, integers, steps, operations, members, components and/or a combination thereof but does not preclude the possibility of the presence or addition of one or more other features, integers, steps, operations, members, components, or a combination thereof. Only the transitional phrases “consisting of” and “consisting essentially of” shall be closed or semi-closed transitional phrases, respectively.

As used herein, the term “can” and “may” are used interchangeably in the present disclosure, and indicate that the referred to element, components, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

As used herein, the term “about” as used herein refers to a measurable value such as an amount, a temporal duration, and the like, is meant to encompass variations of ±20%, ±10%, ±5%, ±1%, or ±0.1% from the specified value, as such variations are appropriate.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “coupled” as used herein refers to two components connected directly to each other or indirectly to another component.

As used herein: μm means micrometer, μm\hat{ }3 or um\hat{ }3 means cubic micrometer, pL means picoliter, nL means nanoliter, and μL (or uL) means microliter.

As used herein, the term “glycan” refers to a polysaccharide, oligosaccharide or monosaccharide. Glycans can be monomers or polymers of sugar residues and can be linear or branched. A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′ sulfo N-acetylglucosamine, etc.).

As used herein, the terms “fucose,” “core fucose,” and “core fucose residue” are used interchangeably and refer to a fucose in α-1,6-position linked to the N-acetylglucosamine.

As used herein, the terms “N-glycan”, “N-linked glycan”, “N-linked glycosylation”, “Fc glycan” and “Fc glycosylation” are used interchangeably and refer to a glycan attached by an N-acetylglucosamine (GlcNAc) linked to the amide nitrogen of an asparagine residue in a Fc-containing polypeptide. The term “Fc-containing polypeptide” refers to a polypeptide, such as an antibody, which comprises an Fc region.

As used herein, the term “glycosylation pattern” and “glycosylation profile” are used interchangeably and refer to the characteristic “fingerprint” of the N-glycan species that have been released from a glycoprotein or antibody, either enzymatically or chemically, and then analyzed for their carbohydrate structure, for example, using LC-HPLC, or MALDI-TOF MS, and the like. See, for example, the review in Current Analytical Chemistry, Vol. 1, No. 1 (2005), pp. 28-57; herein incorporated by reference in its entirety.

As used herein, the term “glycoengineered Fc” when used herein refers to N-glycan on the Fc region that has been altered or engineered either enzymatically or chemically. The term “Fc glycoengineering” as used herein refers to the enzymatic or chemical process used to make the glycoengineered Fc.

As used herein, the term “N-linked initial glycan” refers to a N-linked glycan that is initially attached to a biomolecule before the biomolecule is processed in a reaction conducted according to an embodiment of the present disclosure. The glycan can be a polysaccharide, oligosaccharide or monosaccharide or can be monomers or polymers of sugar residues and can be linear or branched as described herein.

As stated herein, the term “bioconjugate” refers to a molecule formed by a combination of at least two entities, where at least one or all of the at least two entities are biological entities. The entities can all be biological. For example, a bioconjugate can be a polypeptide conjugated with a glycan, a protein conjugated with a glycan, or an antibody or antigen binding fragment thereof conjugated with a glycan. In some examples, a bioconjugate can further comprise a non-biological moiety, such as a chemical entity. The chemical entity can be therapeutic, diagnostic, or providing other functionalities. As used herein, “bioconjugate” and “engineered bioconjugate” can be interchangeable and refer to a conjugate made or obtained by a laboratory-based technology.

The term “antibody drug conjugate” or “immunoconjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the antibody drug conjugate. Additionally, the antibody drug conjugate can be provided in the form of a fusion protein that may be expressed from a polynucleotide encoding the immunoconjugate. As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

The terms “homogeneous”, “uniform”, “uniformly” and “homogeneity” in the context of a glycosylation profile of Fc region are used interchangeably and are intended to mean a single glycosylation pattern represented by one desired N-glycan species, with little or no trace amount of precursor N-glycan, including, for example, less than 95, 96, 97, 98, 99% starting precursor material.

TABLE 1
Listed the four classes of amino acids.

| Side Chain | Amino Acid |
|---|---|
| Basic | Arginine (R), Lysine (K) or Histidine (H) |
| Neutral | Cysteine (C), Tyrosine (Y), Glycine (G), Glutamine (Q), |
| | Threonine (T), Asparagine (N) or Serine (S) |
| Hydrophobic | Isoleucine (I), Leucine (L), Methionine (M), Tryptophan |
| | (W), Proline (P), Valine (V), Phenylalanine (F) or |
| | Alanine (A) |
| Acidic | Aspartic Acid (D) or Glutamic Acid (E) |

As used herein, the terms “IgG”, “IgG molecule”, “monoclonal antibody”, “immunoglobulin”, and “immunoglobulin molecule” are used interchangeably.

As used herein, the term “Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain, (see review M. in Daëron (1997) Annu. Rev. Immunol. 15:203-234). FcRs are reviewed in Ravetch and Kinet (1991) Annu. Rev. Immunol 9:457-92; Capel et al. (1994) Immunomethods 4:25-34; Haas et al. (1995) J. Lab. Clin. Med. 126:330-41). Other FcRs, including those to be identified in the future, are encompassed by the term “FcR” herein. The term also includes the neonatal receptor, FcRn, which is responsible for the transfer of maternal IgGs to the fetus (Guyer et al. (1976) J. Immunol. 117:587 and Kim et al. (1994) J. Immunol. 24:249).

The term “effector function” as used herein refers to a biochemical event that results from the interaction of an antibody Fc region with an Fc receptor or ligand. Exemplary “effector functions” include C1q binding; complement dependent cytotoxicity; Fc receptor binding; antibody-dependent cell-mediated cytotoxicity (ADCC); phagocytosis; downregulation of cell surface receptors (e.g. B cell receptor; BCR), etc. Such effector functions can be assessed using various assays known in the art.

As used herein, the term “Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. (1998) PNAS (USA) 95:652-656.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

The term “hypervariable region”, “HVR”, or “HV”, when used herein refers to the regions of an antibody variable domain which are hypervariable in sequence and/or form structurally defined loops. Generally, antibodies comprise six hypervariable regions: three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)).

“Framework” or “FW” residues are those variable domain residues other than the hypervariable region residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “antigen binding fragment”, as used herein, refers to one or more portions of an antibody that retain the ability to specifically interact with (e.g., by binding, steric hindrance, stabilizing/destabilizing, spatial distribution) an epitope of an antigen. Examples of binding fragments include, but are not limited to, single-chain Fvs (scFv), camelid antibodies (e.g., VHH), disulfide-linked Fvs (sdFv), Fab fragments, F(ab′) fragments, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., Nature 341:544-546, 1989), which consists of a VH domain; and an isolated complementarity determining region (CDR), or other epitope-binding fragments of an antibody.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO93/1161; and Hollinger et al., Proc. Natl. Acad. Sci. USA 90: 6444-6448 (1993).

A “human antibody” is one which possesses an amino acid sequence that corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more HVRs thereof which result in an improvement in the affinity of the antibody for antigen, compared to a parent antibody that does not possess those alteration(s). In one embodiment, an affinity matured antibody has nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “blocking” antibody or an “antagonist” antibody is one which inhibits or reduces biological activity of the antigen it binds. Certain blocking antibodies or antagonist antibodies substantially or completely inhibit the biological activity of the antigen.

An “agonist antibody”, as used herein, is an antibody that mimics at least one of the functional activities of a polypeptide of interest.

A “disorder” is any condition that would benefit from treatment with an antibody of the invention. This includes chronic and acute disorders or diseases including those pathological conditions which predispose the mammal to the disorder in question. Non-limiting examples of disorders to be treated herein include cancer.

The terms “cell proliferative disorder” and “proliferative disorder” refer to disorders that are associated with some degree of abnormal cell proliferation. In one embodiment, the cell proliferative disorder is cancer.

“Tumor” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The terms “cancer” and “cancerous” refer to or describe the physiological condition in mammals that is typically characterized by unregulated cell growth/proliferation. A “tumor” comprises one or more cancerous cells. Examples of cancer include, but are not limited to, carcinoma, lymphoma (e.g., Hodgkin's and non-Hodgkin's lymphoma), blastoma, sarcoma, and leukemia. More particular examples of such cancers include lung cancer, breast cancer, head-and-neck cancer, esophagus cancer, stomach cancer, bladder cancer, pancreatic cancer, colorectal cancer, cervix cancer, endometrial cancer, ovarian cancer, laryngeal cancer, prostate cancer, thyroid cancer and oral cancer.

As used herein, “treatment” refers to clinical intervention in an attempt to alter the natural course of the individual or cell being treated and can be performed either for prophylaxis or during the course of clinical pathology. Desirable effects of treatment include preventing occurrence or recurrence of disease, alleviation of symptoms, diminishment of any direct or indirect pathological consequences of the disease, preventing or decreasing inflammation and/or tissue/organ damage, decreasing the rate of disease progression, amelioration or palliation of the disease state, and remission or improved prognosis. In some embodiments, antibodies of the invention are used to delay development of a disease or disorder.

An “individual” or a “subject” is a vertebrate. In certain embodiments, the vertebrate is a mammal. Mammals include, but are not limited to, farm animals (such as cows), sport animals, pets (such as cats, dogs, and horses), primates, mice and rats. In certain embodiments, the vertebrate is a human.

“Mammal” for purposes of treatment refers to any animal classified as a mammal, including humans, domestic and farm animals, and zoo, sports, or pet animals, such as dogs, horses, cats, cows, etc. In certain embodiments, the mammal is human.

An “effective amount” refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount is also one in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

A “combination” refers to combination therapy would be the amount of the ADC and/or the amount of other biological or chemical drugs that when administered together (either as co-administration and/or co-formulation), either sequentially or simultaneously, on the same or different days during a treatment cycle, have a synergistic effect that is therapeutically effective and more than therapeutically additive.

The term “cytotoxic agent” as used herein refers to a substance that inhibits or prevents the function of cells and/or causes destruction of cells. The term is intended to include radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁰C, and radioactive isotopes of lutetium-177, strontium-89 and samarium (153Sm)), chemotherapeutic agents, and toxins such as small molecule toxins or enzymatically active toxins of bacterial, fungal, plant or animal origin, including synthetic analogs and derivatives thereof.

The term “photodynamic therapy (PDT)’, sometimes called photochemotherapy, is a form of phototherapy involving light and a photosensitizing chemical substance, used in conjunction with molecular oxygen to elicit cell death (phototoxicity). It is used clinically to treat a wide range of medical conditions, including wet age-related macular degeneration, psoriasis, atherosclerosis and has shown some efficacy in anti-viral treatments, including herpes. It also treats malignant cancers including head and neck, lung, bladder, skin and prostate cancer (Wang, S S et al. Cancer Journal. 8 (2): 154-63. 2002). The “photodynamic therapeutic agent” is selected from Photofrin, Laserphyrin, Aminolevulinic acid (ALA), Silicon Phthalocyanine Pc 4, m-tetrahydroxyphenylchlorin (mTHPC), chlorin e6 (Ce6), Allumera, Levulan, Foscan, Metvix, Hexvix, Photochlor, Photosens, Photrex, Lumacan, Visonac, Amphinex, Verteporfin, Purlytin, ATMPn, Zinc phthalocyanine (ZnPc), Protoporphyrin IX (PpIX), Pyropheophorbidea (PPa) or Pheophorbide a (PhA).

A “chemotherapeutic agent” is a chemical compound useful in the treatment of cancer. Examples of chemotherapeutic agents include Monomethyl auristatin E (MMAE), Monomethyl auristatin F (MMAF), mertansine (also called DM1), anthracycline, pyrrolobenzodiazepine, α-amanitin, tubulysin, benzodiazepine, erlotinib (TARCEVA®), Genentech/OSI Pharm.), bortezomib (VELCADE®, Millenium Pharm.), fulvestrant (FASLODEX®, Astrazeneca), sunitinib (SUTENT®, SU11248, Pfizer), letrozole (FEMARA®), Novartis), imatinib mesylate (GLEEVEC®, Novartis), PTK787/ZK 222584 (Novartis), oxaliplatin (ELOXATIN®, Sanofi), leucovorin, rapamycin (Sirolimus, RAPAMUNE®, Wyeth), lapatinib (TYKERB®, GSK572016, GlaxoSmithKline), lonafarnib (SARASAR®, SCH 66336), sorafenib (NEXAVAR®, BAY43-9006, Bayer Labs.), and gefitinib (IRESSA®, Astrazeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine (XELODA®, Roche); and pharmaceutically acceptable salts, acids or derivatives of any of the above.

Also included in this definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON. toremifene; (ii) aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in abherant cell proliferation, such as, for example, PKC-alpha, Ralf and H-Ras; (viii) ribozymes such as a VEGF expression inhibitor (e.g., ANGIOZYME® ribozyme) and a HER2 expression inhibitor; (ix) vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; (x) anti-angiogenic agents such as bevacizumab (AVASTIN®, Genentech); and (xi) pharmaceutically acceptable salts, acids or derivatives of any of the above.

Protein kinase inhibitors include tyrosine kinase inhibitors which inhibit to some extent tyrosine kinase activity of a tyrosine kinase such as an ErbB receptor. Examples of tyrosine kinase inhibitors include EGFR-targeted drugs such as: (i) antibodies which bind to EGFR, including MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBITUX®, Imclone) and reshaped human 225 (H225) (WO 96/40210, Imclone Systems Inc.); antibodies that bind type II mutant EGFR (U.S. Pat. No. 5,212,290); humanized and chimeric antibodies that bind EGFR (U.S. Pat. No. 5,891,996); and human antibodies that bind EGFR, such as ABX-EGF (WO 98/50433); (ii) anti-EGFR antibody conjugated with a cyotoxic agent (EP 659439A2); and small molecules that bind to EGFR including ZD1839 or Gefitinib (IRESSA™; Astra Zeneca), Erlotinib HCl (CP-358774, TARCEVA™; Genentech/OSI) and AG1478, AG1571 (SU 5271; Sugen), quinazolines such as PD 153035,4-(3-chloroanilino) quinazoline, pyridopyrimidines, pyrimidopyrimidines, pyrrolopyrimidines, such as CGP 59326, CGP 60261 and CGP 62706, and pyrazolopyrimidines, 4-(phenylamino)-7H-pyrrolo[2,3-d]pyrimidines, curcumin (diferuloyl methane, 4,5-bis(4-fluoroanilino)phthalimide), tyrphostines containing nitrothiophene moieties; PD-0183805 (Warner-Lambert); antisense molecules (e.g., those that bind to ErbB-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-ErbB inhibitors such as CI-1033 (Pfizer); Affinitac (ISIS 3521; Isis/Lilly); Imatinib mesylate (Gleevac; Novartis); PKI 166 (Novartis); GW2016 (Glaxo SmithKline); CI-1033 (Pfizer); EKB-569 (Wyeth); Semaxanib (Sugen); ZD6474 (AstraZeneca); PTK-787 (Novartis/Schering AG); INC-1C11 (Imclone); or as described in: U.S. Pat. No. 5,804,396; WO 99/09016 (American Cyanamid); WO 98/43960 (American Cyanamid); WO 97/38983 (Warner Lambert); WO 99/06378 (Warner Lambert); WO 99/06396 (Warner Lambert); WO 96/30347 (Pfizer, Inc); WO 96/33978 (Zeneca); WO 96/3397 (Zeneca); and WO 96/33980 (Zeneca).

An “anti-angiogenic agent” refers to a compound that blocks, or interferes with to some degree, the development of blood vessels. The anti-angiogenic factor may, for instance, be a small molecule or antibody that binds to a growth factor or growth factor receptor involved in promoting angiogenesis. An exemplary anti-angiogenic agent is an antibody that binds to Vascular Endothelial Growth Factor (VEGF) such as bevacizumab (AVASTIN®, Genentech).

The term “cytokine” is a generic term for proteins released by one cell population that act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-0; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

The phrase “pharmaceutically acceptable salt,” as used herein, refers to pharmaceutically acceptable organic or inorganic salts of an ADC. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

“Pharmaceutically acceptable solvate” refers to an association of one or more solvent molecules and an ADC. Examples of solvents that form pharmaceutically acceptable solvates include, but are not limited to, water, isopropanol, ethanol, methanol, DMSO, ethyl acetate, acetic acid, and ethanolamine.

The complex N-linked oligosaccharide on each CH2 domain of IgGs is crucial for the structure of the Fc region and thus the interaction with the Fc receptors (Krapp et al. 2003; Woof and Burton 2004). The oligosaccharide chain at IgG-Fc domain contains several N-Acetyl-Glucosamine (GlcNAc) and mannose (Man) residues, and eventually galactose (Gal) and fucose (Fuc) residues as well as sialic acid (Sia or NANA for N-acetylneuraminic acid). A GlcNAc, with or without α1-6 Fuc, is attached to the Asn297. A GlcNAcpi-4 is attached to this first GlcNAc. A manβ1-4 is then found, to which two Manal-6 and Manul-3 arms are attached. Both arms contain an additional GlcNAcβ1-2 to which a Galβ1-4 can be attached or not. Thus, the carbohydrate chain can contain 0, 1 or 2 galactose residues, defining G0, G1, and G2 glycoforms, respectively. Further variations occur, including the presence of a bisecting GlcNAcβ1-4 and the capping of one or both of the terminal galactose residues with a sialic acid or even a Galal-3 residue. The enzymatic cleavage of the Fc-glycan with Endoglycosidases causes the Fc region to deform, and thus, dramatically decrease in IgGs binding to Fcγ receptors (Allhorn et al. 2008). Despite their 37% sequence identity, both EndoS and EndoS2 catalyze the hydrolysis of the β-1,4 linkage between the two N-acetylglucosamines (GlcNAcs) in the core of the N-linked glycan of human IgG. However, in addition to complex type glycans, EndoS2 hydrolyze hybrid and oligomannose structures to a greater extent compared with EndoS (Sjögren et al. 2015).

### Methods for Preparing an Engineered Bioconjugate

Since the introduction of the first antibody therapy in the 1980s, there are more than 240 therapeutic antibodies in clinical trials and the field is steadily expanding (Chan and Carter 2010). The role of the IgG-Fc glycans on antibody functions has gained a huge attention in the growing field of monoclonal therapeutic antibodies. Therefore, to improve the efficacy of the therapeutic antibodies, the major focus is turning towards the engineering the Fc-glycan that specifically interacts with selected Fcγ receptors (Sondermann et al. 2013; Bournazos et al. 2014; Monnet et al. 2014; Quast and Lünemann 2014). Some of the important glycan modifications that dramatically affect the effector functions includes, i) the lack of a core fucose residue attached to the reducing end GlcNAc residue leads to increased affinity for Fcγ RIIIa and thus increased antibody-dependent cytotoxicity (Iidaet et al. 2006); ii) sialic acid rich glycans on IgG that have been claimed to increase the anti-inflammatory response of IgGs through increased interaction with DC-SIGN receptors on dendritic cells and macrophages (Anthony et al. 2008; Anthony and Ravetch 2010; Pincetic et al. 2014); iii) having bisecting GlcNAc induces a strong ADCC as compared to its parental counterpart. The recent improvements in biotechnology tools to control the Fc-glycosylation states of IgG facilitate the development of therapeutic antibodies with predefined glycoforms. Accordingly, the Glycosynthase enzymes of present disclosure are a great advancement in the field of glyco-engineering of peptides, proteins, and antibodies of interest to attach broad range of N-glycans of high mannose, hybrid and complex types for functional and structural studies.

One aspect of the present disclosure provides a method for preparing an engineered bioconjugate. The method comprises contacting a biomolecule with a glycosynthase and a modified glycan thereby obtaining a first engineered bioconjugate. The biomolecule comprises an antibody or antigen binding fragment thereof, a protein, or a peptide, which coupled with a N-linked initial glycan. The modified glycan comprises a substrate moiety and a first reactive moiety, wherein the substrate moiety is configured to interact with the glycosynthase. In some embodiments, contacting the biomolecule with the glycosynthase and the modified glycan comprising contact a plurality of the biomolecules with the glycosynthase and the modified glycan thereby obtaining a plurality of the first engineered bioconjugates. In certain embodiments, the plurality of the first engineered bioconjugates a homogeneity of the plurality of the first engineered bioconjugate is at least or above 80%, 85%, 90%, 95%, or 99%. A high homogeneity indicates less isomer within the plurality of the first engineered bioconjugates and suggests a better consistency and efficacy of the engineered bioconjugates prepared by using the method of the present disclosure. Without wishing to be bound by theories, the glycosynthase and/or the modified glycan of the present disclosure attributes to the favorable high homogeneity.

In some embodiments, the method further comprises contacting the first engineered bioconjugate with a payload conjugate or a salt thereof thereby obtaining a second engineered bioconjugate. In some embodiments, the second engineered bioconjugate is an ADC having a therapeutic agent conjugated with an antibody via the modified glycan. The structure and characteristics of the payload conjugates and the second engineered bioconjugate are described in more detail below.

In some embodiments, contacting the first engineered bioconjugate with a payload conjugate or a salt thereof comprises contacting the first engineered bioconjugate with a first payload conjugate and a second payload conjugate, wherein the first payload conjugate and the second payload conjugate are different. In certain embodiments, the first payload conjugate and the second payload conjugate are different in the respective payloads of the two payload conjugates, the respective reactive moiety units (C) of the two payload conjugates, and/or the respective linker unit (L) of the two payload conjugates.

Glycosylation (deglycosylation and transglycosylation). Contacting a biomolecule with a glycosynthase and a modified glycan, in some embodiments, comprises coupling the modified glycan with the N-linked initial glycan. The modified glycan can be attached to the N-linked initial glycan directly or indirectly. In some embodiments, coupling the modified glycan with the N-linked initial glycan comprise removing the N-linked initial glycan of the biomolecule thereby obtaining a deglycosylated biomolecule; and contacting the deglycosylated biomolecule with the glycosynthase in the presence of the modified glycan.

Removing the N-linked initial glycan of the biomolecule can be removing the N-linked initial glycan entirely or removing parts of the N-linked initial glycan. In the embodiments where only parts of the N-linked initial glycan are removed, the modified glycan will be coupled with the residues of the N-linked initial glycan. In certain embodiments, after the step of removing the N-linked initial glycan, a GlcNAc monosaccharide, which can be fucosylated or non-fucosylated, would be left on the biomolecule, and the modified glycan will be coupled with the GlcNAc monosaccharide.

In some embodiments, removing the N-linked initial glycan of the biomolecule can be conducted by using a glycosynthase of the present disclosure. The glycosynthase of the present disclosure comprises SEQ ID NO. 1 or SEQ ID NO. 2, and the glycosynthase comprises a mutation located within residues 176-186, residues 225-237, residues 273-289 in the sequence of SEQ ID NO. 1 or within residues 178-188, residues 227-239, residues 275-291 in the sequence of SEQ ID NO. 2. The glycosynthase of the present disclosure will be described in more detail below.

Alternatively, the N-linked initial glycan of the biomolecule can be removed by using a glycosynthase different from the glycosynthase of the present disclosure. In certain embodiments, removing the N-linked initial glycan of the biomolecule thereby obtaining a deglycosylated biomolecule comprises, in the absence of the modified glycan, mixing the glycosynthase and the biomolecule at a ratio of from 1:500 to 1:1, from 1:500 to 1:10, from 1:500 to 1:20, from 1:500 to 1:30, from 1:500 to 1:50, from 1:100 to 1:1, from 1:100 to 1:10, from 1:100 to 1:20, from 1:100 to 1:30, from 1:100 to 1:50, from 1:50 to 1:1, from 1:50 to 1:10, from 1:50 to 1:20, or from 1:50 to 1:30. However, the methods of the present disclosure are not so limited. A ratio can vary based on different situations.

In the present disclosure, exemplary glycosynthase enzymes for transglycosylation at core fucosylated or non-fucosylated GlcNAc-acceptor are provided, wherein the core fucosylated or non-fucosylated GlcNAc-acceptor comprising core fucosylated or non-fucosylated GlcNAc-peptides, proteins and IgG Fc domain or fragment thereof.

In some embodiments, two glycosynthase enzyme variants are provided, EndoSd-D232M and EndoSz-D234M, which have the glycosynthase activity enabling the production of homogeneous mAbs remodeling. Besides the previously reported EndoSd from Streptococcus dysgalactiae subsp. Dysgalactiae (NCBI GenBank accession No.: ANI26082.1), we have identified and isolated novel enzymes by protein BLAST database on the EndoS and EndoSd sequences. We selected EndoSz from Streptococcus equi subsp. Zooepidemicus Sz105 (NCBI GenBank accession No.: KIS14581.1) as another candidate despite it is a putative protein. We generated EndoSd and EndoSz mutants according to multiple sequence alignment methodology. The results showed that both mutated enzymes have unexpectedly improved/enhanced glycosynthase activity to conjugate bi-antennary complex-type glycan to mAbs. The mAbs suitable for conjugation are obtained/derived from a wide-range targets of various biomarkers or different IgG types. Here we demonstrated that the conjugation results of OBI-888, Herceptin, Perjeta, Erbitux, Rituxan, OBI-898, Vectibix, Humira, Keytruda and Bavencio were satisfactory. We also demonstrated the enzyme efficiency in the conjugation reaction and compared the ADCC activities between heterogeneous and homogeneous mAbs in the related cell systems.

In some embodiments, the present disclosure provides the glycosynthase enzymes variants, wherein the variants have at least about 80%, 81, 82, 83, 84, 85, 86, 87, 88, 89, 90, 91, 92, 93, 94, 95, 96, 97, 98, 99% sequence and/or structural homology thereto and exhibit improved transglycosylation activity on both fucosylated and non-fucosylated GlcNAc acceptors against broad range of N-glycans of high mannose, hybrid and complex types, wherein the said variants enable efficient transfer of an activated oligosaccharide donors on fucosylated and non-fucosylated GlcNAc acceptors to form new homogenous glycoform of glycopeptide or glycoprotein or therapeutic antibodies. The EndoSz and EndoSd mutants are listed in the following table: In some embodiments, the glycosynthase is as described in the U.S. Pat. No. 11,203,645, filed on Jun. 27, 2019, which is hereby incorporated by reference.

TABLE 2
Listed the EndoSz and EndoSd mutants.

| EndoSz mutant | EndoSd mutant |
|---|---|
| D234E | D232E |
| D234R | D232R |
| D234H | D232H |
| D234M | D232M |
| D234V | D232V |
| D234L | D232L |
| D234F | D232F |
| D234S | D232S |
| D234T | D232T |
| D234Q | D232Q |
| T183Q | T181Q |
| D232Q | D230Q |
| D280Q | D278Q |
| S281Q | S279Q |
| T282Q | T280Q |

Modified glycan. As used herein, a “modified glycan” refers to a glycan modified with a chemical entity. The chemical entity can comprise a polymer and at least one functionality that can be served as a functionalization site or conjugation site for another moiety. Furthermore, the modified glycan comprises a substrate moiety, which is configured to be catalyzed by the glycosynthase as described herein thereby coupling the modified glycan with the biomolecule. In some embodiments, a structure of the substrate moiety can be chosen based on the nature of the glycosynthase. In certain embodiments, the substrate moiety is an oxazoline moiety.

In one embodiment, the modified glycan is a synthetic glycan oxazoline comprising diverse N-glycans of high mannose, hybrid and complex types having the formula:

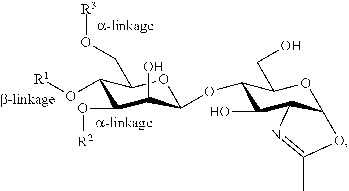

wherein, R¹ is —H or N-acetyl glucosamine attached via β-1,4 linkage and R² and R³ are same or different and are independently selected from the group consisting of:

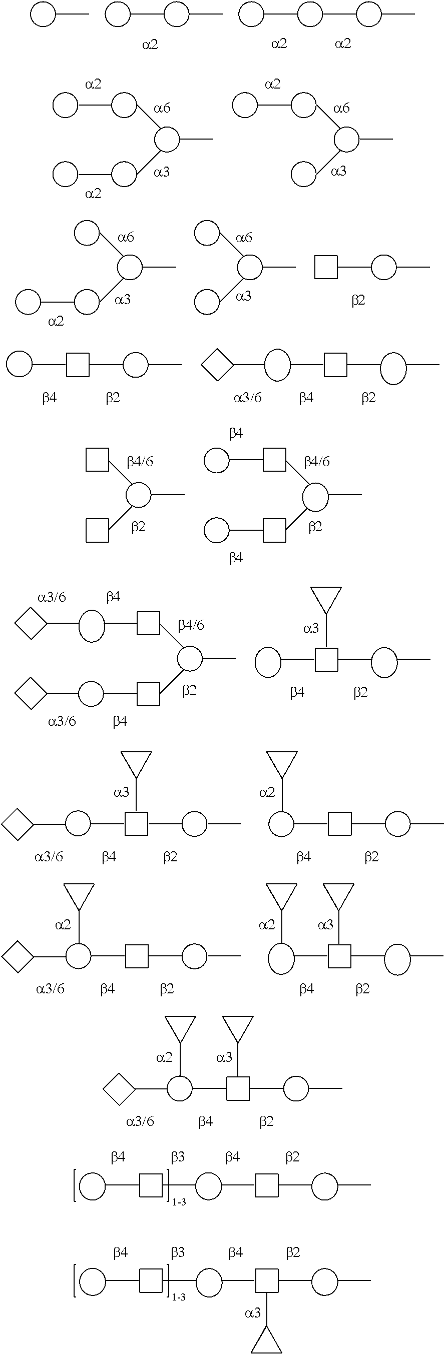

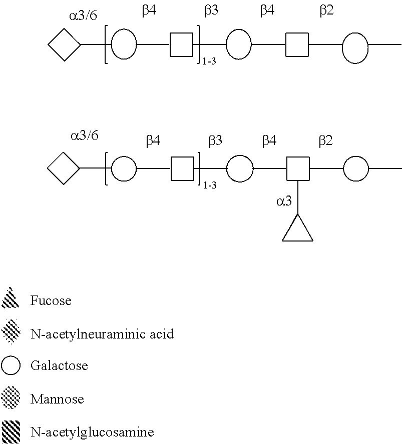

The first reactive moiety of the modified glycan of the present disclosure, in some embodiments, is configured to react with an unsaturated moiety in a biorthogonal reaction, which can be a copper-free click chemistry. In one example, the first reactive moiety comprises an azido group, but the modified glycan is not so limited.

In some embodiments, the modified glycan is a PEGylated glycan modified with a polyethylene glycol (PEG) moiety. Without wishing to be bound by any theories, the PEG moiety provides a better spacing for click reactions to take place, and a PEG moiety is selected for its hydrophilicity facilitating bioreaction and good biocompatibility for clinical uses. In other words, in some embodiments, the PEG moiety can be replaced with other hydrophilic polymeric moieties that exhibit suitable biocompatibility. In certain embodiments, an end of the PEGylated glycan is covalently coupled with the first reactive moiety, for example, and another end of the PEGylated glycan is coupled with the substrate moiety. For example, the first reactive moiety can be coupled with the PEG moiety, and the substrate moiety can be coupled with a glycol moiety of the PEGylated glycan.

While the length of PEG moiety is not limited, in some embodiments, the PEG moiety can comprise 2, 3, 4, 5, 6, 7, 8, 9, 10, 11, 12, 13, 14, 15, 20, 25, 30, 35, 40, 45, 50, 55, 60, 65, 70, or 72 ethylene oxide (OCH₂CH₂) subunits or a range defined by the foregoing endpoints, for example, from 2 to 72, from 2 to 60, from 2 to 50, from 2 to 40, from 2 to 30, from 2 to 20, from 2 to 15, from 2 to 10, from 2 to 5, from 2 to 4, from 3 to 72, from 3 to 65, from 3 to 55, from 3 to 45, from 3 to 35, from 3 to 25, from 3 to 15, from 3 to 10, from 4 to 72, from 4 to 60, from 4 to 50, from 4 to 40, from 4 to 30, from 4 to 20, from 4 to 15, or from 4 to 10 OCH₂CH₂ subunits. In some embodiments, the PEG moiety can be a linear PEG, a branched PEG, or a star PEG. In certain embodiments, the PEG moiety can have 2, 3, 4, 5, 6, 7, 8 arms. In certain embodiments, the PEG moiety can have a molecular weight of 4K Da, 6K Da, 8K Da, 10K Da, or 20K Da.

Biomolecule. In some embodiments, the biomolecule comprises an antibody or antigen binding fragment thereof, and the N-linked initial glycan is located at a constant region of the antibody or antigen-binding fragment. In certain embodiments, the N-linked initial glycan is located at a Fc region of the antibody or antigen-binding fragment, for example, at N297 site of the Fc region. In some embodiments, the biomolecule is an antibody, and one or two N-linked initial glycan is coupled at the N297 site(s) of the Fc region of the antibody.

In some embodiments, the antibody is an IgG, IgM, IgA, IgE, or IgD. A typical IgG consists of two antigen-binding fragments (Fabs), which are connected via a flexible region to a constant region (Fc). The Fab domains are responsible for antigen recognition while the N-glycan at Asn297 of Fc domain interacts with respective Fcγ receptors (such as FcγRIIIa and FcγRIIb) on effector cells and C1q component of the complements that activate the effector functions, including antibody-dependent cellular cytotoxicity (ADCC) and complement-dependent cytotoxicity (CDC). Almost all therapeutic antibodies are N-glycosylated on each of the homodimeric Fc domains at the conserved asparagine residue (N297). These N-linked glycans result in more than 30 different glycoforms and are typical biantennary complex type with considerable structural heterogeneity, in which the core heptasaccharide can be differentially decorated with core fucose (Fuc), bisecting N-acetylglucosamine (GlcNAc), terminal galactose (Gal), and terminal sialic acid (Sia). The composition of N-glycans could influence the Fc domain conformation, therefore, modulating the antibody's stability, pharmacokinetic profile, immunogenicity, effector functions, antibody-mediated inflammation, and complement activation. For example, the absence of the core fucose, as well as the attachment of a bisecting GlcNAc moiety, dramatically enhances the affinity of antibody for the FcγIIIa receptor (FcγRIIIa) on effector cells, resulting in more effective elimination of the target. In addition, the terminal a-2,6-sialylated glycan, which is a minor component of antibodies and the intravenous immunoglobulin (IVIG), is an optimized structure that enhances the anti-inflammatory properties.

In some embodiments, the biomolecule is an anti-Globo series antigen antibody or antigen-binding fragment thereof, an anti-HER2 antibody or antigen-binding fragment thereof, an anti-CD20 antibody or antigen-binding fragment thereof, an anti-TNF-alpha antibody or antigen-binding fragment thereof, an anti-PD-1 antibody or antigen-binding fragment thereof, an anti-PD-L1 antibody or antigen-binding fragment thereof, an anti-TROP2 antibody, an anti-Nectin-4 antibody, or antigen-binding fragment thereof, an anti-EGFR antibody or antigen-binding fragment thereof, an anti-HER3 antibody or antigen-binding fragment thereof, an anti-cMet antibody or antigen-binding fragment thereof, an anti-B7H3 antibody or antigen-binding fragment thereof, an anti-B7H4 antibody or antigen-binding fragment thereof, an anti-VEGF antibody or antigen-binding fragment thereof, an anti-Claudin 18.2 antibody or antigen-binding fragment thereof, an anti-Sirp-Alpha antibody or antigen-binding fragment thereof, an TROP2×HER2 bispecific antibody, or a combination thereof. The Globo series antigen can comprise Globo H, stage-specific embryonic antigen 4 (SSEA-4) or stage-specific embryonic antigen 3 (SSEA-3).

In certain embodiments, the antibody is OBI-888 (Anti-Globo H monoclonal antibody). Exemplary OBI-888 is as described in PCT patent publications (WO2015157629A2 and WO2017062792A1), patent applications, the contents of which are incorporated by reference in its entirety.

In certain embodiments, the antibody is OBI-898 (Anti-SSEA4 monoclonal antibody). Exemplary OBI-898 is as described in PCT patent publication (WO2017172990A1), patent applications, the contents of which are incorporated by reference in its entirety.

In certain embodiments, the antibody is R4702 (Anti-TROP2 monoclonal antibody). Exemplary R4702 is as described in PCT patent publication (WO2022222992A1), patent applications, the contents of which are incorporated by reference in its entirety.

In some embodiments, the biomolecule is selected from, but not limited to, Herceptin (trastuzumab), TX05 (trastuzumab biosimilar), Perjeta (pertuzumab), Erbitux (cetuximab), Rituxan (rituximab), Vectibix (panitumumab), Humira (adalimumab), Keytruda (pembrolizumab) Bavencio (avelumab), and R4702 (anti-TROP2 antibody).

In further aspect, the present disclosure provides a composition of fucosylated or non-fucosylated glyco-engineered antibodies or antigen binding fragments comprising IgG molecules having the same N-glycan structure at each site of the Fc region, wherein the N-glycan is of high mannose, hybrid, and complex types and is selected from the group consisting of:

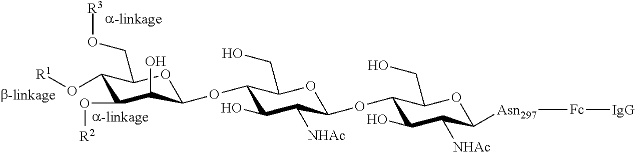

Wherein, R¹ is —H or N-acetyl glucosamine attached via β-1,4 linkage and R² and R³ are same or different and are independently selected from the group consisting of:

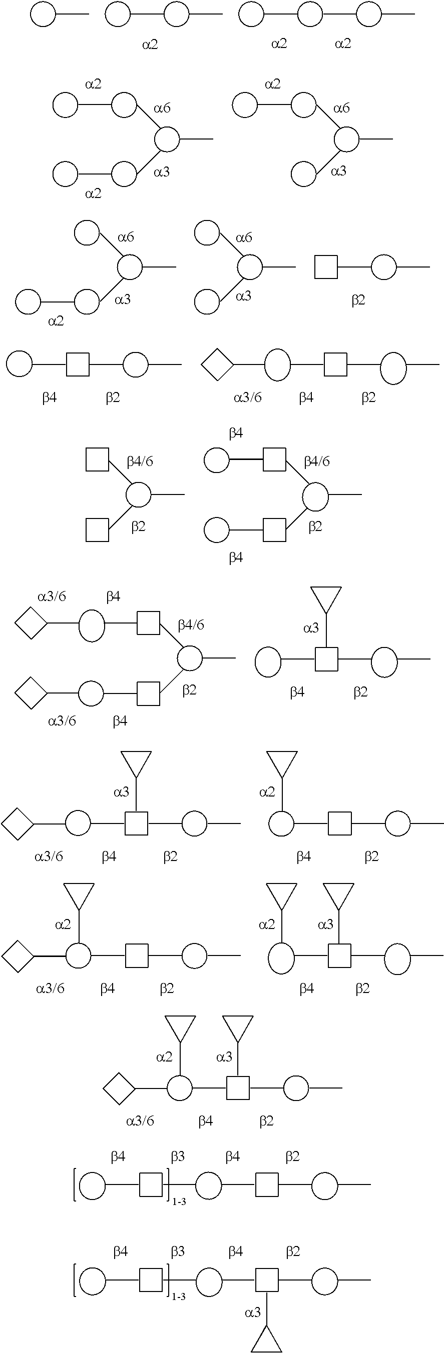

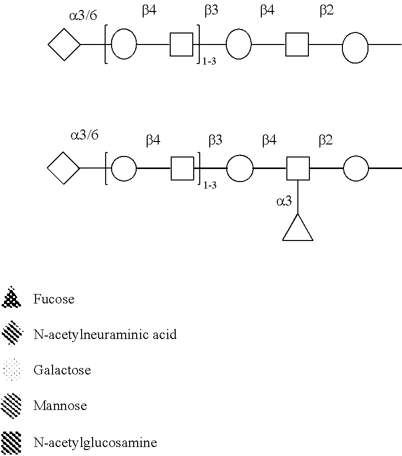

In another aspect, the present disclosure provides the engineered bioconjugate with unexpectedly improved effector functions such as bindings to FcγIIIA, ADCC and regulates immune response, as compared to non-modified antibodies.

### Payload Conjugate (Linker-Drug Conjugate)

Another aspect of the present disclosure provides a payload conjugate, which can be a therapeutic conjugate or a linker-drug compound represented by the following formula:

C-L-D,

wherein C is a second reactive moiety, configured to react with the first reactive moiety of the modified glycan in a biorthogonal reaction; wherein L is a linker unit comprising a hydrophilic moiety; and wherein D is a payload. The payload conjugate is configured to couple the payload with the biomolecule covalently via the first reactive moiety of the modified glycan.

Second reactive moiety Unit (C). In order to react with the first reactive moiety of the modified glycan, the second reactive moiety of the payload conjugate, in some embodiments, comprises an unsaturated moiety, which can react with an azido moiety thereby resulting in a triazole moiety. The unsaturated moiety can be, but not limited to, an alkene moiety or an alkyne moiety. In some embodiments, the second reactive moiety is a bioorthogonal group that is a non-native and nonperturbing chemical group. Some specific examples of the second reactive moiety include, but not limited to a dibenzocyclooctyne group (DBCO), a bicyclononyne (BCN), a cyclic alkyne, a maleimide group, a α,β-unsaturated carbonyl group, or a sulfonyl pyrimidine. Nevertheless, the method of the present disclosure is not so limited. Other functional groups that are suitable for a biorthogonal reaction with the first reactive moiety of the modified glycan can also be selected for the payload conjugation.

Linker unit (L). The hydrophilic moiety of the linker (L) can be selected based on the hydrophilicity and biocompatibility for clinical uses. In certain embodiments, the hydrophilic moiety comprises a second polyethylene glycol (PEG) moiety. In some embodiments, the PEG moiety can be replaced with other hydrophilic polymeric moieties that exhibit suitable biocompatibility.

While the length of PEG moiety is not limited, in some embodiments, the PEG moiety can comprise 2, 3, 4, 5, 6, 7, 8, 9, 10, 11, 12, 13, 14, 15, 20, 25, 30, 35, 40, 45, 50, 55, 60, 65, 70, or 72 ethylene oxide (OCH₂CH₂) subunits or a range defined by the foregoing endpoints, for example, from 2 to 72, from 2 to 60, from 2 to 50, from 2 to 40, from 2 to 30, from 2 to 20, from 2 to 15, from 2 to 10, from 2 to 5, from 2 to 4, from 3 to 72, from 3 to 65, from 3 to 55, from 3 to 45, from 3 to 35, from 3 to 25, from 3 to 15, from 3 to 10, from 4 to 72, from 4 to 60, from 4 to 50, from 4 to 40, from 4 to 30, from 4 to 20, from 4 to 15, or from 4 to 10 OCH₂CH₂ subunits. In some embodiments, the PEG moiety can be a linear PEG, a branched PEG, or a star PEG. In certain embodiments, the PEG moiety can have 2, 3, 4, 5, 6, 7, 8 arms. In certain embodiments, the PEG moiety can have a molecular weight of 4K Da, 6K Da, 8K Da, 10K Da, or 20K Da.

The linker, in some embodiments, can further comprise a cleavable moiety and a spacer. The cleavable moiety is configured to release the payload from the second engineered conjugate in vivo. Therefore, some functionalities that can be catalyzed or digested by enzymes commonly existing in a target environment in vivo can be selected. The enzyme can be a protease, such as matrix metalloproteinase 2 or matrix metalloproteinase 9, or a glycosidase. In certain embodiments, the cleavable moiety is a protease sensitive peptide, including but not limited to, Val-Cit, Val-Ala, Phe-Lys, Glu-Val-Cit, Glu-Val-Ala, Glu-Gly-Cit, Glu-Gly-Ala, Gly-Gly-Phe-Gly, Gly-Gly-Val-Cit, Gly-Gly-Val-Ala. In other embodiments, the cleavable moiety is a glycosidase sensitive sugar unit, including but not limited to, glucuronic acid, iduronic acid, or galactose.

In some embodiments, the spacer can be an aromatic group, including but not limited to, a 1,4-phenyl group, a 2,5-pyridyl group, a 3,6-pyridyl group, a 2,5-pyrimidyl group, 2,5-thienyl group, or an amino methylene, such as —NH—CH₂—.

In some embodiments, the linker unit comprises a formula of:

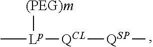

wherein (PEG)m is a PEG moiety, and m is an integer selected from 2 to 72, Q^{SP} is a spacer comprising an aromatic group or amino methylene, Q^{CL} is a cleavable moiety and is configured to link to the payload, L^{P} is a connector unit configured to link to the second reactive moiety.

In some embodiments, the PEG moiety comprises a formula of

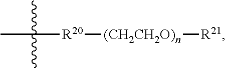

wherein the wavy line indicates the site of covalent attachment to L^{P}, wherein R²⁰ is a PEG attachment unit, wherein the PEG attachment unit is —C(O)—, —O—, —S—, —NH—, —C(O)O—, alkyl-C(O)—NH—, alkyl-NH—C(O)—, alkyl-CO₂—, alkyl-S—, or

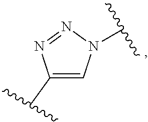

wherein R²¹ is a PEG capping unit; wherein the PEG capping unit is selected from H, SO₃H, PO₃H₂, a sugar derivative, C₁-C₁₀ (hetero) alkyl group, C₃-C₁₀ (hetero) cycloalkyl group, C₂-C₁₀ alkyl-NH₂, C₁-C₁₀ alkyl-COOH, C₂-C₁₀ alkyl-NH(C₁-C₃ alkyl), C₂-C₁₀ alkyl-N(C₁-C₃ alkyl)₂, and n is selected from 2 to 72, 4 to 72, or 8 to 72.

In a specific example, the linker unit (L) has a structure of:

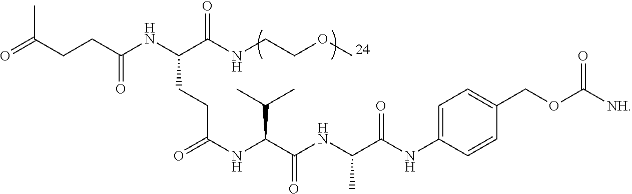

Payload (D). The payload can be a therapeutic agent or a diagnostic agent. In some embodiments, the diagnostic agent can be used for imaging.

In some embodiments, the therapeutic agent can be a toxin, a cytokine, a growth factor, a radionuclide, a hormone, an anti-viral agent, an anti-bacterial agent, an immunoregulatory, an immunostimulatory agent, an anti-tumor agent, a chemotherapeutic agent, or a combination thereof.

In some embodiments, the toxin is at least one selected from the group moiety consisting of: a pyrrolobenzodiazepine (e.g. PBD); an auristatin (e.g. MMAE, MMAF); a maytansinoid (e.g. maytansine, DM1, DM4, DM21); a duocarmycin; a nicotinamide phosphoribosyltransferase (NAMPT) inhibitor; a tubulysin; an enediyne (e.g. calicheamicin); an anthracycline derivative (PNU) (e.g. doxorubicin); a pyrrole-based kinesin spindle protein (KSP) inhibitor; a cryptophycin; a drug efflux pump inhibitor; a sandramycin; an amanitin (e.g. α-amanitin); and a camptothecin (e.g. exatecan, deruxtecan).

In certain embodiments, the chemotherapeutic agent is Topoisomerase inhibitor, which comprises Topoisomerase I inhibitor and Topoisomerase II inhibitor. In certain embodiments, the chemotherapeutic agent is Topoisomerase I inhibitor, which is Camptothecin (CPT) or Non-camptothecins, selected from Irinotecan, Topotecan, Camptothecin, Rubitecan, MLN576, Exatecan, Belotecan, Seconeolitsine, SN-38, Genz-644282, Betulinic acid, β-Lapachone, Karenitecin, Gimatecan, Namitecan, Edotecarin, SW044248, LMP744, T-2513, Podocarpusflavone A, Indimitecan, Lurtotecan, TP3011 or 10-hydroxycamptothecin.

Exatecan could be selected in the payload (D) as an example and Linker-drug compound (i.e., the payload conjugate) may be further represented by the following formula:

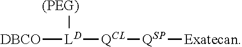

In one embodiment, the structure of linker-drug compound (i.e., the payload conjugate) is represented by the following formula:

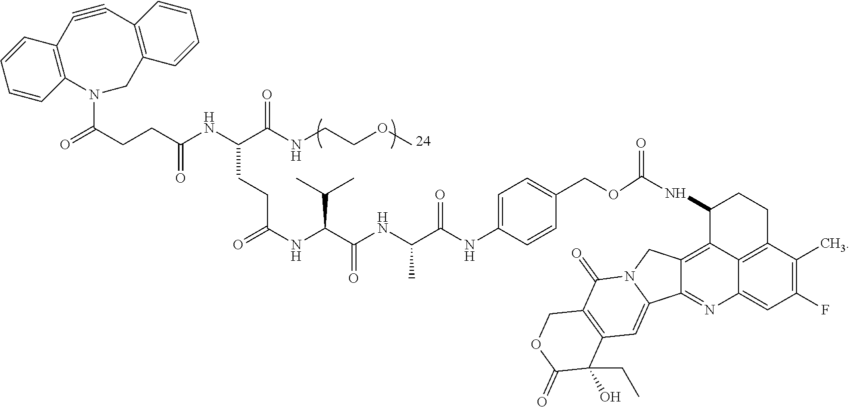

In one embodiment, the structure of linker-drug compound (i.e., the payload conjugate) is represented by the following formula:

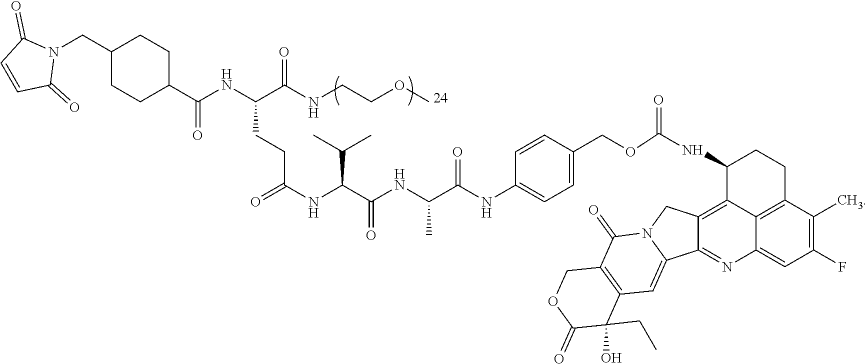

### Engineered Bioconjugate

Another aspect of the present disclosure provides an engineered bioconjugate. The engineered bioconjugate comprises a biomolecule and a modified glycan, coupled with the biomolecule, wherein the modified glycan comprises (i) a first polyethylene glycol (PEG) moiety and (ii) a first reactive moiety or a resultant moiety thereof from a biorthogonal reaction; wherein the biomolecule comprises an antibody or antigen binding fragment thereof, a protein, or a peptide. In some embodiments, the first PEG moiety and the first reactive moiety are as those described herein.

In some embodiments where the biomolecule comprises the antibody or antigen binding fragment thereof, the modified glycan is coupled with the antibody or antigen-binding fragment thereof at a Fc region thereof. In certain embodiments, the modified glycan is located at a Fc region of the antibody or antigen-binding fragment, for example, at N297 site of the Fc region. In some embodiments where the biomolecule is an antibody, one or two N-linked initial glycan is coupled at the N297 site(s) of the Fc region of the antibody, for example, with a GlcNAc monosaccharide at the N297 site. The GlcNAc monosaccharide can be fucosylated or non-fucosylated.

In some embodiments, the engineered bioconjugate further comprises a payload moiety, wherein the payload moiety is coupled with the modified glycan via the resultant moiety. In such embodiments, the payload-to-biomolecule ratio (e.g., a drug-to-antibody ratio) is 2 to 1, 4 to 1, 5 to 1, 6 to 1, 7 to 1, 8 to 1, 9 to 1, or 10 to 1.

In certain embodiments, the payload moiety has a structure represented by the following formula:

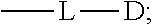

wherein L is a linker unit comprising a hydrophilic moiety and is linked to the resultant moiety, and D is a payload. In some embodiments, the linker unit (L) and the payload (D) are as those described herein. In some embodiments, the engineered bioconjugate is an engineered bioconjugate prepared by the method for preparing an engineered bioconjugate according to an embodiment of the present disclosure.

In some embodiments, the resultant moiety can be a moiety resulted from a biorthogonal reaction or click reaction between the first reactive moiety and the second reactive moiety as described herein. In certain embodiments, the resultant moiety comprises a triazole moiety. In certain embodiments, the resultant moiety can comprise a DBCO-derived moiety or a maleimide-derived moiety.

In some embodiments, the present disclosure provides a plurality of engineered bioconjugates, each is of the engineered bioconjugate according to an embodiment of the present disclosure, wherein a homogeneity of the plurality of engineered bioconjugates is at least or above 80%, 85%, 90%, 95%, or 99%.

In some embodiments, the engineered bioconjugate comprises a first payload moiety and a second payload moiety, wherein the first payload moiety and the second payload moiety are different. In certain embodiments, the first payload moiety and the second payload moiety are different in the respective payloads of the two payload conjugates, the respective reactive moiety units (C) of the two payload conjugates, and/or the respective linker unit (L) of the two payload conjugates.

In certain embodiments where the first payload moiety and the second payload moiety are different in the respective payloads of the two payload conjugates, the payload of the first payload moiety can be a therapeutic agent and the payload of the second payload moiety can be an imaging agent (e.g., an imaging probe). In other embodiments, the payload of the first payload moiety can be a first therapeutic agent and the payload of the second payload moiety can be a second therapeutic agent.

The engineered bioconjugate of the present disclosure can include those with utility for anticancer activity. In particular embodiments, the engineered bioconjugate includes an antibody conjugated, i.e. covalently attached by a linker, to a therapeutic agent/payload forming an ADCs. The therapeutic agent when not conjugated to an antibody has a cytotoxic or cytostatic effect. The biological activity of the therapeutic agent/payload is thus modulated by conjugation to an antibody. In certain embodiments, the ADCs of the present disclosure is able to selectively deliver an effective dose of a cytotoxic agent to a tumor whereby a lower efficacious dose may be achieved.

In some embodiments that the engineered bioconjugate is an ADC, the ADC may be represented by the following formula:

Ab-(L-D)ₙ or a pharmaceutically acceptable salt or solvate thereof,
wherein: Ab is an antibody which binds TROP2, HER2, Nectin-4, HER3, cMet, B7H3, B7H4, VEGF, Claudin 18.2, Sirp-Alpha, or which binds to one or more tumor-associated antigens or cell-surface receptors; D is a drug unit; L is linker; and n is the drug-to-antibody ratio (DAR) and ranging from 10 to 1.

Suitable exemplary linkers for the ADC are described in, for example, U.S. Pat. No. 7,595,292 (WO2005/007197). The entire content directed to linkers is hereby incorporated by reference herein. The linker, L, attaches the antibody to a drug moiety/payload through covalent bond(s), not comprising a disulfide group. The linker is a bifunctional or multifunctional moiety which can be used to link one or more drug moieties/payloads (D) and an antibody unit (Ab) to form ADCs of Formula I. ADCs can be conveniently prepared using a linker having reactive functionality for binding to the Drug and to the Antibody. A cysteine thiol, or an amine, e.g. N-terminus or amino acid side chain such as lysine, of the antibody (Ab) can form a bond with a functional group of a linker reagent, drug moiety/payload or drug-linker reagent.

The linkers are preferably stable extracellularly. Before transport or delivery into a cell, the ADC is preferably stable and remains intact, i.e. the antibody remains linked to the drug moiety/payload. The linkers are stable outside the target cell and may be cleaved at some efficacious rate inside the cell. An effective linker will: (i) maintain the specific binding properties of the antibody; (ii) allow intracellular delivery of the conjugate or drug moiety/payload; (iii) remain stable and intact, i.e. not cleaved, until the conjugate has been delivered or transported to its targeted site; and (iv) maintain a cytotoxic, cell-killing effect or a cytostatic effect of the maytansinoid drug moiety/payload. Stability of the ADC may be measured by standard analytical techniques such as mass spectroscopy, HPLC, and the separation/analysis technique LC/MS.

In another embodiment, the ADC specifically binds to TROP2, HER2, Nectin-4, HER3, cMet, B7H3, B7H4, VEGF, Claudin 18.2, and Sirp-Alpha. The ADC may inhibit growth of tumor cells which express TROP2, HER2, Nectin-4, HER3, cMet, B7H3, B7H4, VEGF, Claudin 18.2, and Sirp-Alpha.

Another aspect includes diagnostic and therapeutic uses for the compounds and compositions disclosed herein.

Another aspect is a method for killing or inhibiting the proliferation of tumor cells or cancer cells comprising treating the cells with an amount of an engineered conjugate or an ADC according to an embodiment of the present disclosure, or a pharmaceutically acceptable salt or solvate thereof, being effective to kill or inhibit the proliferation of the tumor cells or cancer cells.

Another aspect includes methods of treating a disease or disorder characterized by the overexpression of TROP2, HER2, Nectin-4, HER3, cMet, B7H3, B7H4, VEGF, Claudin 18.2, and Sirp-Alpha in a patient with the engineered conjugate or the ADC.

Another aspect includes methods of making, methods of preparing, methods of synthesis, methods of conjugation, and methods of purification of the engineered conjugate or the ADC, and the intermediates for the preparation, synthesis, and conjugation of the ADCs.

### Pharmaceutical Formulations

Another aspect of the present disclosure provides a pharmaceutical composition comprising a plurality of engineered bioconjugates of the present disclosure and a pharmaceutically acceptable carrier. Pharmaceutical formulations comprising an engineered conjugate of the present disclosure may be prepared for storage by mixing the antibody having the desired degree of purity with one or more optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, including, but not limited to, those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nano-capsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the immunoglobulin of the invention, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated immunoglobulins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The amount of antibody in the pre-lyophilized formulation is determined by taking into account the desired dose volumes, mode(s) of administration etc. Where the protein of choice is an intact antibody (a full-length antibody), from about 2 mg/mL to about 50 mg/mL, preferably from about 5 mg/mL to about 40 mg/mL and most preferably from about 20-30 mg/mL is an exemplary starting protein concentration. The protein is generally present in solution. For example, the protein may be present in a pH-buffered solution at a pH from about 4-8, and preferably from about 5-7. Exemplary buffers include histidine, phosphate, Tris, citrate, succinate and other organic acids. The buffer concentration can be from about 1 mM to about 20 mM, or from about 3 mM to about 15 mM, depending, for example, on the buffer and the desired isotonicity of the formulation (e.g. of the reconstituted formulation). The preferred buffer is histidine in that, as demonstrated below, this can have lyoprotective properties. Succinate was shown to be another useful buffer.

The lyoprotectant is added to the pre-lyophilized formulation. In preferred embodiments, the lyoprotectant is a non-reducing sugar such as sucrose or trehalose. The amount of lyoprotectant in the pre-lyophilized formulation is generally such that, upon reconstitution, the resulting formulation will be isotonic. However, hypertonic reconstituted formulations may also be suitable. In addition, the amount of lyoprotectant must not be too low such that an unacceptable amount of degradation/aggregation of the protein occurs upon lyophilization. Where the lyoprotectant is a sugar (such as sucrose or trehalose) and the protein is an antibody, exemplary lyoprotectant concentrations in the pre-lyophilized formulation are from about 10 mM to about 400 mM, and preferably from about 30 mM to about 300 mM, and most preferably from about 50 mM to about 100 mM.

The ratio of protein to lyoprotectant is selected for each protein and lyoprotectant combination. In the case of an antibody as the protein of choice and a sugar (e.g., sucrose or trehalose) as the lyoprotectant for generating an isotonic reconstituted formulation with a high protein concentration, the molar ratio of lyoprotectant to antibody may be from about 100 to about 1500 moles lyoprotectant to 1 mole antibody, and preferably from about 200 to about 1000 moles of lyoprotectant to 1 mole antibody, for example from about 200 to about 600 moles of lyoprotectant to 1 mole antibody.

In preferred embodiments of the invention, it has been found to be desirable to add a surfactant to the pre-lyophilized formulation. Alternatively, or in addition, the surfactant may be added to the lyophilized formulation and/or the reconstituted formulation. Exemplary surfactants include nonionic surfactants such as polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethyl glycol, polypropyl glycol, and copolymers of ethylene and propylene glycol (e.g. Pluronics, PF68, etc.). The amount of surfactant added is such that it reduces aggregation of the reconstituted protein and minimizes the formation of particulates after reconstitution. For example, the surfactant may be present in the pre-lyophilized formulation in an amount from about 0.001-0.5%, and preferably from about 0.005-0.05%.

In certain embodiments of the invention, a mixture of the lyoprotectant (such as sucrose or trehalose) and a bulking agent (e.g., mannitol or glycine) is used in the preparation of the pre-lyophilization formulation. The bulking agent may allow for the production of a uniform lyophilized cake without excessive pockets therein etc.

Other pharmaceutically acceptable carriers, excipients, or stabilizers such as those described in Remington's Pharmaceutical Sciences 16th edition, Osol, A. Ed. (1980) may be included in the pre-lyophilized formulation (and/or the lyophilized formulation and/or the reconstituted formulation) provided that they do not adversely affect the desired characteristics of the formulation. Acceptable carriers, excipients or stabilizers are nontoxic to recipients at the dosages and concentrations employed and include additional buffering agents; preservatives; co-solvents; antioxidants including ascorbic acid and methionine; chelating agents such as EDTA; metal complexes (e.g. Zn-protein complexes); biodegradable polymers such as polyesters; and/or salt-forming counterions such as sodium.

The pharmaceutical compositions and formulations described herein are preferably stable. A “stable” formulation/composition is one in which the antibody therein essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period.

The formulations to be used for in vivo administration must be sterile. This is readily accomplished by filtration through sterile filtration membranes, prior to, or following, lyophilization and reconstitution. Alternatively, sterility of the entire mixture may be accomplished by autoclaving the ingredients, except for protein, at about 120° C. for about 30 minutes, for example.

After the protein, lyoprotectant and other optional components are mixed together, the formulation is lyophilized. Many different freeze-dryers are available for this purpose such as Hull50© (Hull, USA) or GT20© (Leybold-Heraeus, Germany) freeze-dryers. Freeze-drying is accomplished by freezing the formulation and subsequently subliming ice from the frozen content at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will mainly dictate the time required for drying, which can range from a few hours to several days (e.g., 40-60 hours). A secondary drying stage may be carried out at about 0-40° C., depending primarily on the type and size of container and the type of protein employed. However, it was found herein that a secondary drying step may not be necessary. For example, the shelf temperature throughout the entire water removal phase of lyophilization may be from about 15-30° C. (e.g., about 20° C.). The time and pressure required for secondary drying will be that which produces a suitable lyophilized cake, dependent, e.g., on the temperature and other parameters. The secondary drying time is dictated by the desired residual moisture level in the product and typically takes at least about 5 hours (e.g. 10-15 hours). The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial. As a general proposition, lyophilization will result in a lyophilized formulation in which the moisture content thereof is less than about 5%, and preferably less than about 3%.

At the desired stage, typically when it is time to administer the protein to the patient, the lyophilized formulation may be reconstituted with a diluent such that the protein concentration in the reconstituted formulation is at least 50 mg/mL, for example from about 50 mg/mL to about 400 mg/mL, more preferably from about 80 mg/mL to about 300 mg/mL, and most preferably from about 90 mg/mL to about 150 mg/mL. Such high protein concentrations in the reconstituted formulation are considered to be particularly useful where subcutaneous delivery of the reconstituted formulation is intended. However, for other routes of administration, such as intravenous administration, lower concentrations of the protein in the reconstituted formulation may be desired (for example from about 5-50 mg/mL, or from about 10-40 mg/mL protein in the reconstituted formulation). In certain embodiments, the protein concentration in the reconstituted formulation is significantly higher than that in the pre-lyophilized formulation. For example, the protein concentration in the reconstituted formulation may be about 2-40 times, preferably 3-10 times and most preferably 3-6 times (e.g. at least three fold or at least four fold) that of the pre-lyophilized formulation.

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution. The diluent optionally contains a preservative. Exemplary preservatives have been described above, with aromatic alcohols such as benzyl or phenol alcohol being the preferred preservatives. The amount of preservative employed is determined by assessing different preservative concentrations for compatibility with the protein and preservative efficacy testing. For example, if the preservative is an aromatic alcohol (such as benzyl alcohol), it can be present in an amount from about 0.1-2.0% and preferably from about 0.5-1.5%, but most preferably about 1.0-1.2%. Preferably, the reconstituted formulation has less than 6000 particles per vial which are >10 μm in size.

### Therapeutic Applications

The engineered bioconjugate described herein may be used for treating a patient having a cancer. The method of the treatment comprises administering to a patient in need an effective amount of the engineered bioconjugate or the pharmaceutical composition described herein. Examples of the cancers include, but are not limited to, cancers associated with and/or expressing Globo series antigens, including, but not limited to, Globo H, SSEA-4, SSEA-3; cancers associated with and/or expressing HER2, TROP2, Nectin-4, HER3, cMet, B7H3, B7H4, VEGF, Claudin 18.2, or Sirp-Alpha.

The subject to be treated by the methods described herein can be a mammal, more preferably a human. Mammals include, but are not limited to, farm animals, sports animals, pets, primates, horses, dogs, cats, mice and rats. A human subject who needs the treatment may be a human patient having, at risk for, or suspected of having cancer, which includes, but not limited to, sarcoma, skin cancer, leukemia, lymphoma, brain cancer, lung cancer, breast cancer, oral cancer, esophagus cancer, stomach cancer, liver cancer, bile duct cancer, pancreas cancer, colon cancer, kidney cancer, cervix cancer, ovary cancer and prostate cancer. A subject having cancer can be identified by routine medical examination. Particularly, the cancer is Globo series antigen expressing cancer.

In certain embodiments, the cancer is a breast cancer.

Further, the engineered bioconjugate described herein may be used for treating a patient having an autoimmune or inflammatory disease. The method of the treatment comprises administering to the patient an effective amount of a glycoengineered antibody or a pharmaceutical composition described herein. Examples of the autoimmune or inflammatory disease include, but are not limited to, rheumatoid arthritis, juvenile rheumatoid arthritis, systemic lupus erythematosus (SLE), Wegener's disease, inflammatory bowel disease, idiopathic thrombocytopenic purpura (ITP), thrombotic thrombocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Crohn's disease, ulcerative colitis, gastritis, Hashimoto's thyroiditis, ankylosing spondylitis, hepatitis C-associated cryoglobulinemic vasculitis, chronic focal encephalitis, bullous pemphigoid, hemophilia A, membranoproliferative glomerulonephritis, adult and juvenile dermatomyositis, adult polymyositis, chronic urticaria, primary biliary cirrhosis, neuromyelitis optica, Graves' dysthyroid disease, bullous pemphigoid, membranoproliferative glomerulonephritis, Churg-Strauss syndrome, asthma, psoriatic arthritis, dermatitis, respiratory distress syndrome, meningitis, encephalitis, uveitis, eczema, atherosclerosis, leukocyte adhesion deficiency, juvenile onset diabetes, Reiter's disease, Behcet's disease, hemolytic anemia, atopic dermatitis, Wegener's granulomatosis, Omenn's syndrome, chronic renal failure, acute infectious mononucleosis, HIV and herpes-associated disease, systemic sclerosis, Sjogren's syndrome and glomerulonephritis, dermatomyositis, ANCA, aplastic anemia, autoimmune hemolytic anemia (AIHA), factor VIII deficiency, hemophilia A, autoimmune neutropenia, Castleman's syndrome, Goodpasture's syndrome, solid organ transplant rejection, graft versus host disease (GVHD), autoimmune hepatitis, lymphoid interstitial pneumonitis (HIV), bronchiolitis obliterans (non-transplant), Guillain-Barre Syndrome, large vessel vasculitis, giant cell (Takayasu's) arteritis, medium vessel vasculitis, Kawasaki's Disease, and polyarteritis nodosa.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Other Aspects of the Present Disclosure

In a separate aspect, the present disclosure provides a remodeling method of core fucosylated or non-fucosylated GlcNAc-peptide, protein, and IgG or IgG-Fc fragment, wherein the method comprising: providing peptide/protein/antibody-GlcNAc acceptor or Fc fragment and reacting with an activated oligosaccharide donors under the catalysis of Streptococcus dysgalactiae subsp. Dysgalactiae and Streptococcus equi subsp. Zooepidemicus Sz105 glycosynthase enzymes, and thereby preparing substantially, essentially, and/or pure glycoforms of pre-existing peptides, proteins and monoclonal antibodies having heterogeneous glycosylation states.

In further aspect, the present disclosure provides method of using Glycosynthase enzymes for glycan remodeling of therapeutic IgG or Fc fragment thereof, wherein the method comprising:
A. Treating natural or recombinant core fucosylated or non-fucosylated therapeutic IgG or IgG-Fc fragment carrying heterogeneous N-glycans with Endoglycosidase (e.g., wild type EndoS2) together with or without bacterial alpha fucosidases to hydrolyze bond between two reducing end GlcNAc residues to form core fucosylated or non-fucosylated GlcNAc-IgG acceptor;
B. Transferring the wide range of predefined oligosaccharide building units in the form of activated oligosaccharide donors to core fucosylated or non-fucosylated GlcNAc-IgG to reconstitute natural beta 1,4 linkage through transglycosylation using Streptococcus dysgalactiae subsp. Dysgalactiae and Streptococcus equi subsp. Zooepidemicus Sz105 Glycosynthase enzymes, thereby attaching the predefined oligosaccharide to remodel core fucosylated or non-fucosylated IgG or Fc fragment thereof.

### EXAMPLES

Embodiments of the invention will be further illustrated with the following specific examples. One skilled in the art would appreciate that these specific examples are for illustration only and that other modifications and variations are possible without departing from the scope of the invention. For example, the enzyme variants of the invention may be used to glycoengineer any glycoproteins or glycopeptides, including antibodies. The specific examples described herein use anti-CD20 antibodies. However, one skilled in the art would appreciate that other glycoproteins or antibodies may also be used in a similar manner.

### Materials

Monoclonal anti-Globo H antibody, OBI-888 was produced according to our previous procedure disclosed in PCT patent publications (WO2015157629A2 and WO2017062792A1). Monoclonal anti-SSEA4 antibody, OBI-898 was produced according to our previously disclosed procedure in the PCT patent publication (WO2017172990A1). The commercial antibodies Herceptin (trastuzumab), Perjeta (pertuzumab), Erbitux (cetuximab), Rituxan (rituximab), Vectibix (panitumumab), Humira (adalimumab), Keytruda (pembrolizumab) and Bavencio (avelumab). were purchased from:

| | Herceptin | Perjeta | Erbitux | Rituxan | Vectibix | Humira | Keytruda | Bavencio |
|---|---|---|---|---|---|---|---|---|
| Brand | Roche | Roche | MerckSerono | Roche | Amgen | AbbVie | MSD Ireland | Merck |
| Lot | N7208B06 B3066 | H0239B09 | 245011 | H0229B13 | 1080265 | 83347XH04 | 7302614A13 | 04150132280583 |

Biantennary glycan, Sialylated complex type N-glycan (NSCT), was purchase from Tokyo Chemical Industry Co., Ltd. (Tokyo, Japan, D4065) and NSCT-oxazoline were synthesized according to previously reported (Noguchi, M. et al. (2012) Helvetica chimica acta 95: 1928-1936). Other glycans (M3, G0, and G2) and Bf-α-fucosidase was produced according to previous papers (Tsai, T. I. et al. (2017) ACS Chem. Biol. 12: 63-72; Fairbanks, A. J. (2013) Pure Appl. Chem. 85, 1847-1863).

### Example 1: Cloning, Overexpression and Purification of EndoSd-D232M and EndoSz-D234M and Mutants

The genes of EndoSd and EndoSz from Streptococcus dysgalactiae subsp. Dysgalactiae (ANI26082.1) and Streptococcus equi subsp. Zooepidemicus Sz105 (KIS14581.1) were used for this study. The signal peptides were deleted in both enzymes at N-terminal. To enhance transglycosylation activity we aligned the EndoSd and EndoSz protein sequence to EndoS-D233Q (Huang, W. et al. (2012) J. Am. Chem. Soc. 134: 12308-12318) and found the relative position is D232 and D234 for EndoSd and EndoSz, respectively. We decided to mutate the relative position D to M. Therefore, the gene encoding amino acids 20-1067 of EndoSd-D232M and 20-1011 of EndoSz-D234M were synthesized and sub-cloned into pGEX-4T-1 with 5′-BamHI and 3′-XhoI restriction sites. For the purification purpose, we inserted an additional six histidine at the C-terminal of EndoSd-D232M and EndoSz-D234M for affinity Ni-NTA column. Other mutates used in this investigation were generated by site-directed mutagenesis. Related primers were designed based on mutated sites. Taking EndoSd-D232M and EndoSz-D234M as template vectors, the mutated vectors were amplified by Pfu DNA polymerase (Protech). Then, the template vectors (methylated DNA) were digested by DpnI (Promega) for 2 hours (37° C.). The mutated vectors were transformed to the DH5α competent cell for selection. All mutants were confirmed by DNA sequencing.

All vectors were transformed into BL21 (DE3) and cultured at 37° C. in TB medium containing ampicillin antibiotic (50 μg/mL). The proteins were induced by 0.2 mM isopropyl-β-D-thiogalactopyranoside (IPTG) while the cell density OD600 reached 0.6. After 5 hours, the cells were harvested at 25° C. by centrifugation (BACKMAN/JLA-8.1, 9000 g) for 15 minutes. The cell pellet was resuspension with wash buffer containing 50 mM MOPS pH 7.0, 300 mM NaCl and 10 mM imidazole (100 mL buffer/1 L cell pellet) for the homogenizer (NanoLyzer N-10) to break the cell. After 60 min/12,000 g (BACKMAN/JA-10) centrifugation at 4° C. and discard pellet, the supernatant was mixed with Ni-NTA resin (Roche) and gentle rocked overnight at 4° C. for protein binding completely. The resin was loaded onto an open column and washed non-bound protein with wash buffer until the concentration of non-bound protein was less than 1 mg/mL (defined by Bradford assay, Thermo). The bound protein was eluted with elute buffer containing 50 mM MOPS pH 7.0, 300 mM Nacl and 250 mM imidazole. The eluted fraction was dialysis to a storage buffer containing 50 mM MOPS pH 6.7 and concentrated using TFF (Millipore lab scale) by 30 kDa cutoff cassette. The final samples were assayed by SDS-PAGE and Braford for detecting MW and concentrates, respectively.

### Example 2: Deglycosylation of OBI-888 by EndoS-WT and Bf-α-Fucosidase to Generate mAb-GlcNAc and mAb-GlcNAc(F)

The OBI-888 and Herceptin monoclonal antibody (10 mg) were incubated with EndoS (10 μg) in a 25 mM sodium citrate buffer (pH 6.5) and 100 mM NaCl at 37° C. for 4 h. The complete cleavage of Fc N-glycans was analyzed by 4-12% gradient SDS-PAGE.

The N-glycans of OBI-888 (10 mg) were digested by incubation with EndoS-WT (10 μg) and Bf-α-fucosidase (10 mg) in the Tris-HCl buffer (pH 7.4) at 37° C. for 16 hours to generate OBI-888-GlcNAc. The commercial antibodies Herceptin (trastuzumab), Perjeta (pertuzumab), Erbitux (cetuximab), Rituxan (rituximab), Vectibix (panitumumab), Humira (adalimumab), Keytruda (pembrolizumab) and Bavencio (avelumab) (10 mg) used the same procedure as OBI-888 except temperature at 30° C. The complete cleavage of Fc N-glycans was analyzed by 4-12% gradient SDS-PAGE. Fucosylated mAb-GlcNAc (mAbs-GlcNAc-F) were produced by only EndoSz wild type in similar condition with 4 hours incubation time.

### Example 3: Tansglycosylation of Glycans to mAb-GlcNAc and mAb-GlcNAc-F

In general procedures, mAb-GlcNAc/mAb-GlucNAc-F (5 mg) was incubated with glycan-oxa with molar ratio 1:20 and 1:150 (mAb-GlcNAc:NSCT-oxa) by EndoSz-D234M (167 μg) or EndoSd-D232M (1002 μg), respectively, at 30° C. for 20 minutes in a MOPS buffer (50 mM, pH 6.7), to final volume 500 μL. There were some slight modifications according to experiment purposes and designs (see result section). HLPC was employed to monitor the transglycosylation efficiency.

### Example 4: Purification of Deglycosylated and Homogeneous mAb

The reaction mixture was applied to a HiTrap Protein-A HP (5 mL, GE) prepack column which pre-equilibrated with PBS buffer. The non-bound contaminations were washed by two steps pH gradient, PBS (pH 7.4) buffer and glycine-HCl (pH 5.0) buffer, with five times of column volume in each step. Sodium citrate (pH 3.0) was employed to elute bound antibody. The eluted fractions were immediately neutralized with Tris-HCl buffer (1M, pH 9.0) to pH 7.4 and dialyzed to the storage buffer containing 50 mM MOPS (pH 6.7) for mAb-GlcNAc(F) and 5 mM Histidine and 150 mM Nacl for mAb-G2S2, respectively, with 30 kDa cutoff dialysis cassette (Thermo) overnight at 4° C. All samples were concentrated by Amicon centrifugation membrane (30 kDa cutoff, Millipore) and storage at 4° C. [mAb-GlcNAc(F)] or −80° C. [mAb-G2S2(F)].

### Example 5: LC/MS/MS of Glycopeptide Analysis

Samples were first processed for buffer exchange into ddH₂O by 10 kDa cut-off Amicon Ultra-0.5 device. Samples were denatured in 0.1% RapiGest SF solution/50 mM triethylammonium bicarbonate (TEABC), reduced with 5 mM Dithiothreitol (DTT) at 60° C. for 30 minutes, then alkylated using 15 mM iodoacetamide in dark at room temperature for 30 minutes. The resulting samples were subjected to in-solution digestion with trypsin (trypsin:sample protein=1:30) in 50 mM TEABC at 37° C. overnight. After digestion, the samples were acidified with 0.5% trifluoroacetic acid (TFA) (v/v) and incubated at 37° C. for 45 minutes. The acid treated samples were centrifuged at 4° C., 14000 rpm for 30 minutes to precipitate the hydrolytic RapiGest SF by-product. The samples were analyzed with Thermo Q-Exactive mass spectrometer (Thermo Scientific) coupled with Ultimate 3000 RSLC system (Dionex). The LC separation was performed using the C18 column (Acclaim PepMap RSLC, 75 μm×150 mm, Thermo) with mobile phase A: 0.1% FA (Formic acid) and B: 95% ACN (acetonitrile)/0.1% FA and Table 3 listed the analysis solvent gradient.

TABLE 3
Solvent gradient of LC

| Time (min) | A % | B % | Flow (μL/min) |
|---|---|---|---|
| 0 | 99 | 1 | 0.25 |
| 5.5 | 99 | 1 | 0.25 |
| 45 | 75 | 25 | 0.25 |
| 48 | 40 | 60 | 0.25 |
| 50 | 20 | 80 | 0.25 |
| 60 | 20 | 80 | 0.25 |
| 65 | 99 | 1 | 0.25 |
| 70 | 99 | 1 | 0.25 |

Full MS scan was performed with m/z range of 300 to 2000 and the ten most intense ions from MS scan were selected for MS/MS scans.

### Example 6: Enzymatic Conjugation Assay by HPLC

The glycosynthase activity was analyzed by HPLC (Waters e2695) using 2.1×150 mm UPLC Glycoprotein Amide Column (Waters) under two different buffers (Buffer A, ddH₂O/0.3% v/v HFIP (1,1,1,3,3,3-Hexafluoro-2-propanol), 0.1% v/v TFA; Buffer B, ACN, 0.3% v/v HFIP, 0.1% v/v TFA) with the gradient as Table 4.

TABLE 4
Solvent gradient of HPLC

| Time | Flow (mL/min) | A (%) | B (%) |
|---|---|---|---|
| 0.00 | 0.20 | 15 | 85 |
| 0.50 | 0.20 | 15 | 85 |
| 1.00 | 0.20 | 33 | 67 |
| 17.0 | 0.20 | 38.6 | 61.4 |
| 20.0 | 0.20 | 38.6 | 61.4 |
| 21.0 | 0.20 | 15 | 85 |
| 30.0 | 0.20 | 15 | 85 |

Before experiment the column was washed by 50% ACN and 50% of ddH₂O for 30 minutes and equilibrated with 15% buffer A and 85% buffer B until the system pressure was stable. After ensuring the baseline stability by injection water blank, the process was started by 2 μL sample injection. In the process duration (29 mins), the flow rate was 0.2 mL/min under the column temperature of 65° C. and sample tray temperature of 5° C.

### Example 7: ADCC Assay of the Engineered Antibody

The ADCC activity was analyzed by ADCC reporter bioassay complete kit (Promega, G7015) using luciferase reporter cells. The related cell lines MCF7W (OBI-888), SKBR-3 (Herceptin, Perjeta), BxPC3 (Erbitux) and Raji (Rituxan) were selected for the analysis. Cell lines shared with the same procedures. Target cells were seeded at 96-well cell culture plate and incubated overnight at 37° C. in a humidified 5% CO₂ incubator. The culture medium was replaced with serial dilution of homogeneous antibody and the corresponding antibody standard in triplicate. In each well, ADCC bioassay effector cells were added. Ratio of effector cell to target cell was 3:1. We performed induction for 6 hours and then added Bio-Glo luciferase assay buffer. After 15 minutes, luminescence (RLU, relative light unit) was determined using microplate reader (SpectraMax L, Molecular Devices, Sunnyvale, CA). The fold change of luminescence induction was calculated by the ratio of relative light unit (RLU) (induced) to RLU (no antibody control). EC₅₀ was determined by plotting x (concentration in μg/mL)-y (the induction of fold change) and fitting the data in a 4PL nonlinear regression model by PRISM 6 Software. Relative potency was estimated by parallel line analysis using Gen5 Microplate Reader and Imager Software (BioTek Instruments).

### Example 8: Cleavage and Conjugation of Herceptin

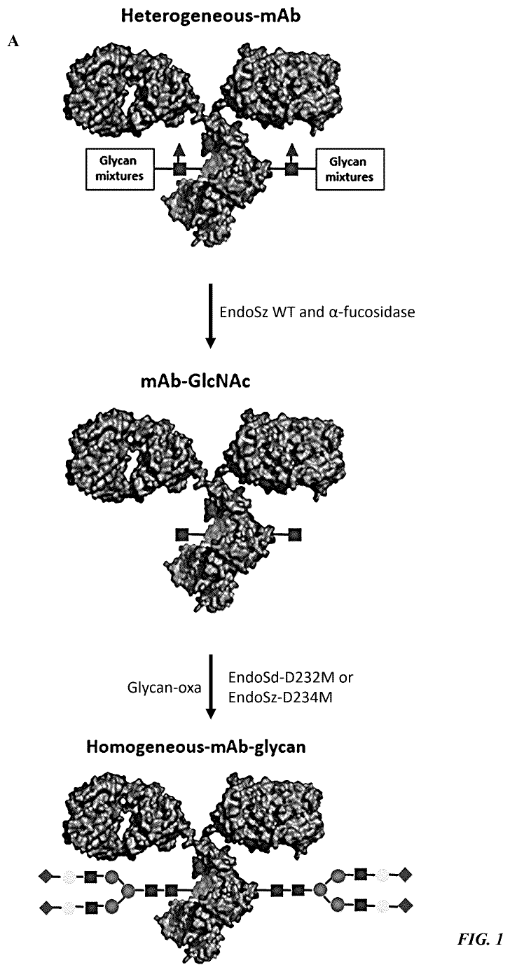

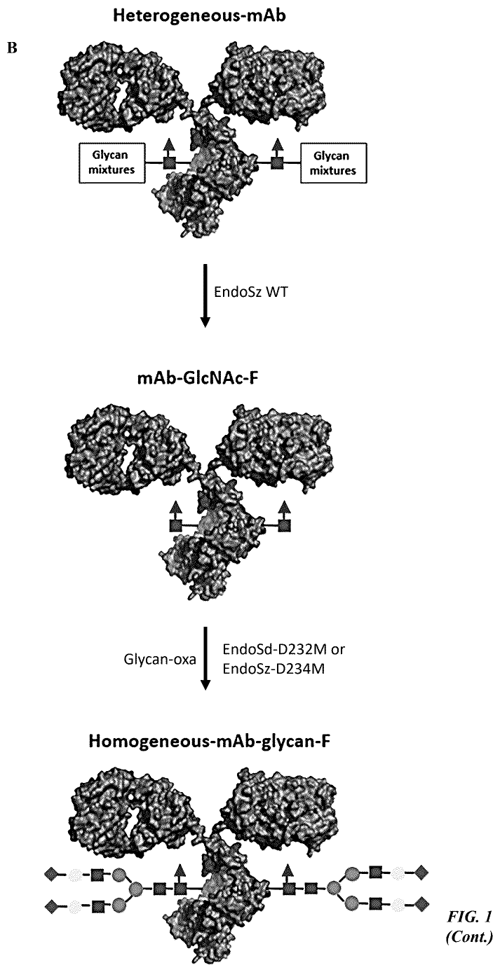

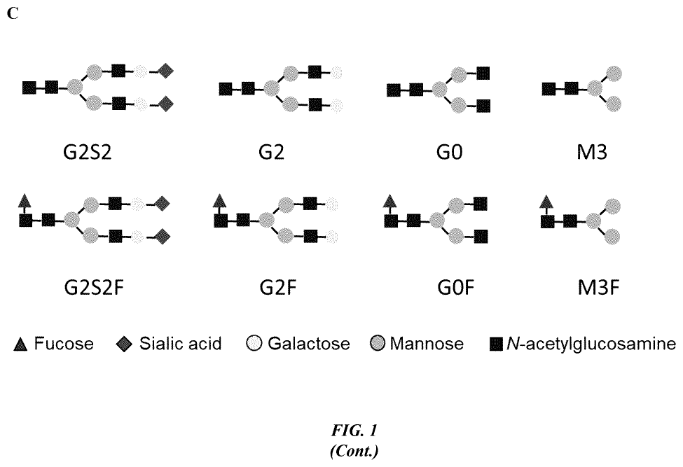

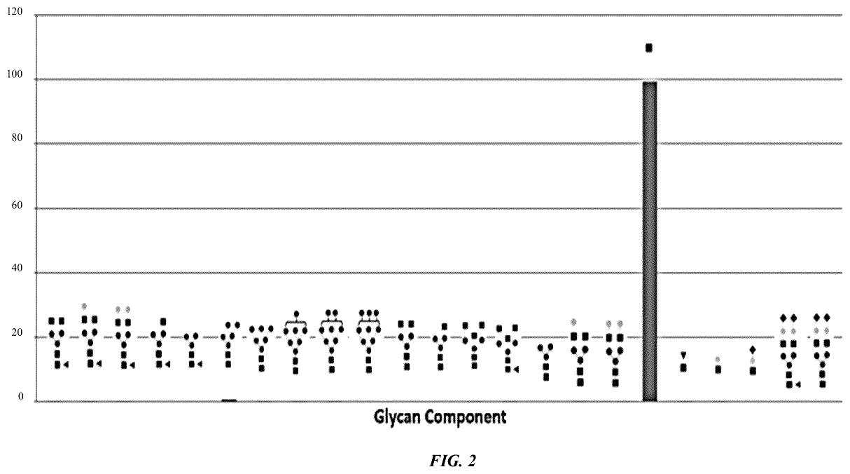

The antibody produced by CHO cell always contains heterogeneous glycans on the N297 position of Fc. To generate homogeneous mAbs and enhance ADCC, enzymatic modification of glycan cleavage and transglycosylation are essential steps in homogeneous platform (FIG. 1). For the glycan cleavage, it has reported that EndoSd-WT hydrolyzed biantennary glycans, but it is not a general chitinase (Shadnezhad, A. et al. (2016) Future Microbiol 11: 721-736). To illustrate the ability of EndoSz-WT, it was used to hydrolyze native Herceptin and detected the N-glycan profile. The result showed EndoSz-WT could hydrolyzed biantennary hybrid and high mannose glycans. It was generated >99% Herceptin with only one N-acetylglycosamine (Herceptin-GlcNAc) on N297 by EndoSz-WT and α-fucosidase enzyme (FIG. 2). However, for the mAb containing glycosylated Fab, we combined additional enzyme, EndoH or EndoM (Shadnezhad, A. et al. (2016) Future Microbiol 11: 721-736; Kadowaki, S. et al. (1990) Agric. Biol. Chem. 54: 97-106), in the cleavage step for the glycan removal completely.

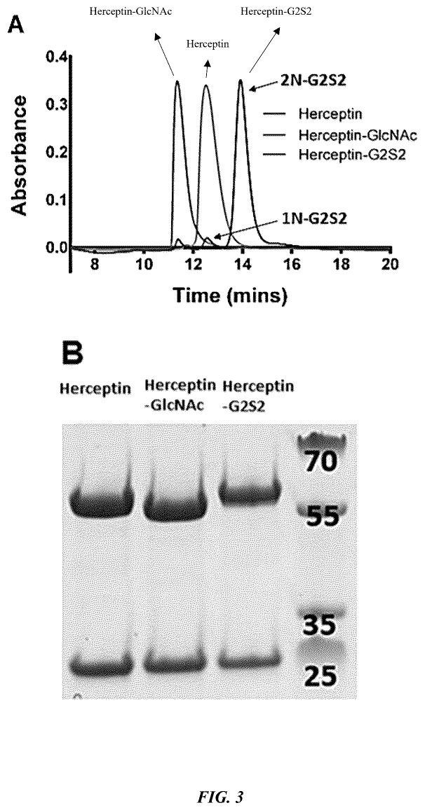

ENGases also have glycan conjugation function with proper mutations (Huang, W. et al. (2012) J. Am. Chem. Soc. 134: 12308-12318; Li., T., Tong, et al. (2016) J. Biol. Chem. 291: 16508-16518). We have established the HPLC method with amide column for precisely monitoring the transglycosylation process. As the example shown in FIG. 3, native Herceptin had retention time at 12.5 minutes. After EndoSz-WT and α-fucosidase cleavage, the retention time of Herceptin with one GlcNAc (Herceptin-GlcNAc) was shifted to 11.4 minute and had the calculated molecular mass MW=145,572 Da by intact mass analysis. In the transglycosylation step of oxazoline sialylated complex type N-glycan (NSCT-oxa) to Herceptin-GlcNAc (demonstrated by EndoSz-D234M), it is interesting to note that the column could clearly identify hemi-glycosylated Herceptin (Herceptin-1N-G2S2) and fully-glycosylated Herceptin (Herceptin-2N-G2S2) that could not be distinguished in the SDS-PAGE. Herceptin-1N-G2S2 was found in the retention time at 12.6 minute with MW=147,574 Da, whereas Herceptin-2N-G2S2 had retention time at 13.9 minute with M=149,576 Da. The HPLC method is able to apply to all mAbs. Therefore, we used HPLC assay for further investigation.

### Example 9: Transglycosylation Investigation of EndoSz-D234M and EndoSd-D232M

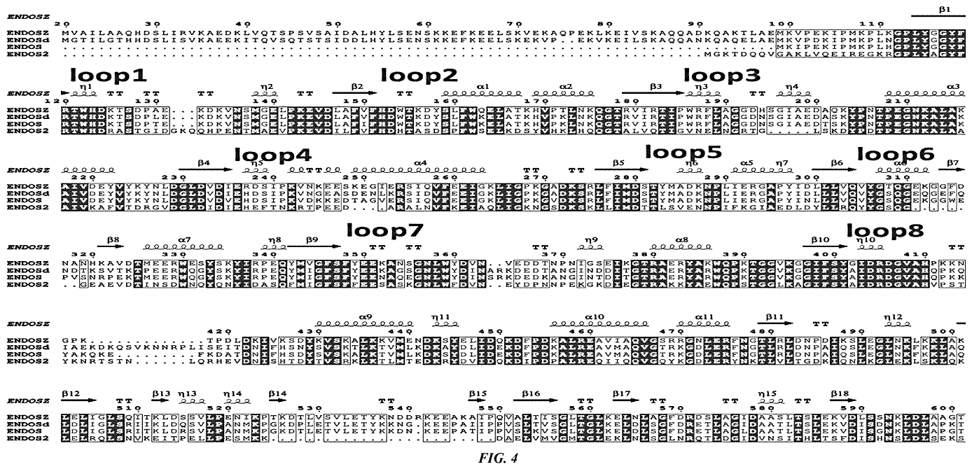

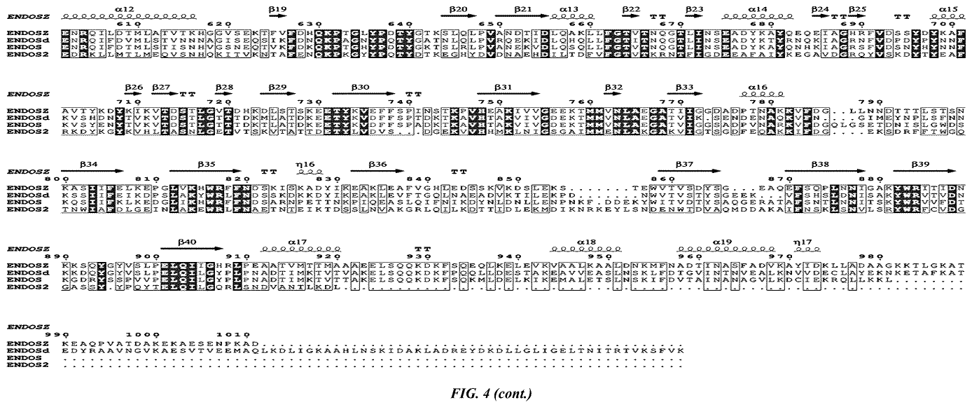

Transglycosylation is the most important step in homogeneous platform that decides the quality of homogeneous mAbs. According to previous report (Li., T., Tong, et al. (2016) J. Biol. Chem. 291: 16508-16518), EndoS2 employed D184M mutation had high transglycosylation activity. We first generated EndoSz-D234M and EndoSd-D232M for the transglycosylation investigation by multiple sequence alignment (FIG. 4). In general, a higher sugar ratio would generate higher conjugation efficiency. However, in practice, decreasing sugar amount to save cost and processing with a reasonable time frame were the goals for process optimization. Herceptin-GlcNAc and NSCT-oxa were used as an investigate model to evaluate the transglycosylation activity of EndoSz-D234M and EndoSd-D232M within 60 minutes and expected the formation of >90% Herceptin-2N-G2S2.

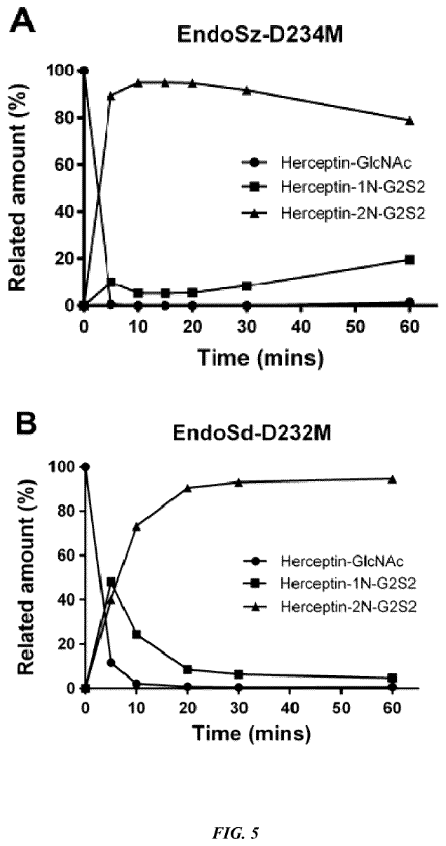

Starting with NSCT-oxa/Herceptin molar ratio of 40:1, >90% Herceptin-2N-G2S2 was obtained with EndoSz-D234M. Decrease the amount of NSCT-oxa/Herceptin molar ratio of 30:1, >90% Herceptin-2N-G2S2 can be reached at 10 minutes. With 20:1 of NSCT-oxa/Herceptin ratio, Herceptin-2N-G2S2 reached 89.31% at 5 minutes, reached 91.85% at 10 minutes and stayed until 20 minutes before deglycosylating (FIG. 5A). With 10:1 of NSCT-oxa/Herceptin (molar ratio), 62.61% of Herceptin-2N-G2S2 were obtained at 5 minutes, 61.48% at 10 minutes and deglycosylation started. To determine the limit of NSCT-oxa usage, increase amount of NSCT-oxa/Herceptin to 15:1 (molar ratio). 80.8% of Herceptin-2N-G2S2 was attained in 5 minutes, and deglycosylation started at 10 minutes. Therefore, the final condition of transglycosylation with EndoSz-D234M enzyme was NSCT-oxa:antibody=20:1 (molar ratio) with 20 minutes reaction time.

The time-dependent graph of EndoSz-D234M (FIG. 5A) clearly illustrated the enzyme behavior. The enzyme quickly bound to Fc and conjugated glycan to N297 position of Fc as Herceptin-GlcNAc decreased almost to 0% within five minutes and most of fully glycosylated antibody was found. However, the high efficiency transglycosylation enzyme also contributes to hydrolysis activity. In all trials, deglycosylation occurred while the reaction reached the peak efficiency of transglycosylation and stayed for a period of time (20 minutes), which indicated the importance of controlling the reaction time in the process. Notably, the data showed decreasing percentage of Herceptin-2N-G2S2 accompanied by increasing major percentage of Herceptin-1N-G2S2 and minor percentage of Herceptin-GlcNAc, implying the enzyme has priority to select the targets in the hydrolysis reaction.

In contrast, applying the final transglycosylation condition of EndoSz-D234M onto EndoSd-D232M enzyme showed inconsistent results. Only 46.06% Herceptin-2N-G2S2 was generated by EndoSd-D232M with molar ratio 20:1 (NSCT-oxa/antibody), which demonstrated a better glycosynthase activity of EndoSz-D234M compares to that of EndoSd-D232M. To obtain higher fully glycosylated antibody, increasing amount of EndoSd-D232M and NSCT-oxa were employed to enhance transglycosylation efficiency. When five times of EndoSd-D232M enzyme was used, only 60% Herceptin-2N-G2S2 was produced. When NSCT-oxa amount was increased to 80:1 (molar ratio), an unstable result in the range of 80-90% Herceptin-2N-G2S2 was produced. Finally, 150:1 (molar ratio) of NSCT-oxa gave a stable and repeatable data. The time dependent graph (FIG. 5B) showed that the EndoSd-D232M slowly transferred NSCT-oxa to N297 (Fc region) despite larger amount of substrate was used. In 5 minutes, the Herceptin-1N-G2S2 (~50%) has larger amount than Herceptin-2N-G2S2 (~40%). Herceptin-2N-G2S2 reached to 80% at 10 minutes and 94% at 20 minutes. The deglycosylation was not found within 60 minutes.

Besides using Herceptin-GlcNAc as acceptor, we also studied transglycosylation activity of fucosylated Herceptin-GlcNAc (Herceptin-GlcNAc-F) that can potentially apply in ADC. Using the best transglycosylation conditions described previously, 94.29% and 94.75% Herceptin-2N-G2S2F were obtained by EndoSz-D234M and EndoSd-D232M, respectively (Table 5). This demonstrated that two enzymes have transglycosylation activity on fucosylated substrate.

TABLE 5
The transglycosylation results of EndoSz-D234M
and EndoSd-D232M in different acceptors.

| Enzyme | Acceptor | GlcNAc(F) | 1N-G2S2 | 2N-G2S2 |
|---|---|---|---|---|
| EndoSz- | Herceptin-GlcNAc | 3.35% | 4.8% | 91.85% |
| D234M | Herceptin-GlcNAc-F | 1.72% | 4.00% | 94.29% |
| EndoSd- | Herceptin-GlcNAc | 0% | 2.11% | 97.88% |
| D232M | Herceptin-GlcNAc-F | 0% | 3.17% | 94.75% |

In conclusion, EndoSz-D234M has a better transglycosylation activity than EndoSd-D232M. EndoSz-D234M stable produced >90% Herceptin-2N-G2S2 (Herceptin-2N-G2S2F) with only 20:1 molar ratio (NSCT-oxa:antibody), whereas EndoSd-D232M needs 7.5 folds of substrate

### Example 10: Transglycosylation Investigation of EndoSz and EndoSd Mutates

The previous reports that several mutant sites, such as EndoS-D233Q and EndoS2-D184M could increase transglycosylation activity for glycan (Huang, W. et al. (2012) J. Am. Chem. Soc. 134: 12308-12318; Li., T., Tong, et al. (2016) J. Biol. Chem. 291: 16508-16518). Recently, Shivatare et al. reported that the mutation of EndoS2-T138Q increases better activity than EndoS2-D184M (Shivatare, S. S. et al. (2018) Chem. Commun. 54, 6161-6164). According to multiple sequence alignment (FIG. 4), the equivalent positions T183, D232, D234, D280, S281 and T282 of EndoSz, and T181, D230, D232, D278, S279 and T280 of EndoSd were selected as targets for site-directed mutagenesis investigation, in which EndoSz D234 and EndoSd D232 sites were generated by several different types, including positive/negative charge and polar/non-polar. The transglycosylation activity was assayed using the best conditions described previously.

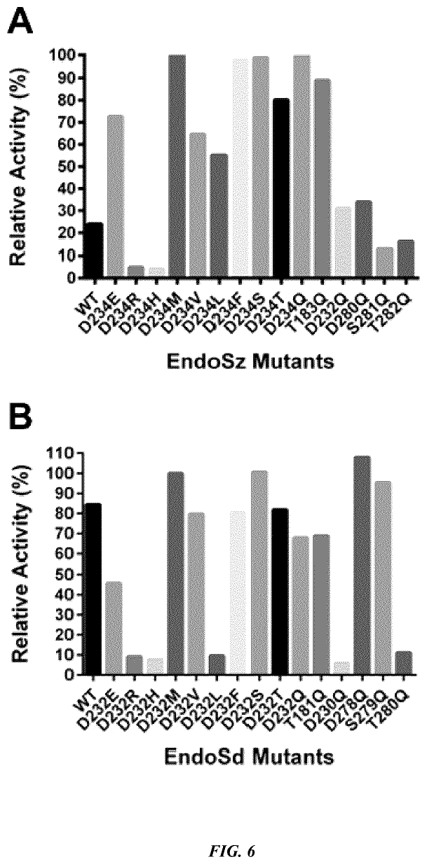

The activity assay results of EndoSz mutants (FIG. 6A) showed that EndoSz-D234M has highest activity (set to 100%) and EndoSz-D234Q (99.9%), EndoSz-D234S (98.8%) and D234F (98.6%) have competitive high activities. In contrast, EndoSz-D234R (4.9%) and EndoSz-D234H (4.5%) have low activities. EndoSz wild type also has sight transglycosylation activity (24.3%). Beside the D234 position, EndoSz-T183Q (89.1%), EnodSz-D232Q (31.4%), EndoSz-D280Q (34.0%), EndoSz-S281Q (12.9%) and EndoSz-T282Q (16.3%) had no significant increasing transglycosylation activity compared to EndoSz-D234M.

In EndoSd mutants (FIG. 6B), EndoSd-D232M (set to 100%), EndoSd-D232S (100.8%) and EndoSd-D278Q (108.0%) have equally high transglycosylation activity, whereas EndoSd-D232R (9.2%) and EndoSd-D232H (8.1%) have low activities. The EndoSd wild type had relatively high transglycosylation activity (84%).

In conclusion, EndoSz-D234M and EndoSd-D278Q were shown to exhibit relatively better transglycosylation activity on Herceptin-GlcNAc to produce homogeneous Herceptin bearing with NSCT-oxa at the Fc region.

### Example 11: Conjugation Investigation of EndoSz-D234M on Various Sugars

The homogeneous platform was designed to conjugate various glycans on Fc region of Herceptin. Glycans, M3, G0 and G2, were used for the investigation by EndoSz-D234M. The results showed all of the glycans were successfully conjugated onto the Fc region with 20:1 molar ratio (NSCT-oxa:antibody). G0 and G2 except M3 reached to >90% fully glycosylated Herceptin (Table 6).

TABLE 6
The transglycosylation results of EndoSz-D234M
in different acceptor with different glycans.

| Acceptor | Glycan type | GlcNAc(F) | 1N-Glycan | 2N-Glycan |
|---|---|---|---|---|
| Herceptin- | M3 (40eq) | 1.11% | 6.51% | 92.37% |
| GlcNAc | G0 | 0.91% | 2.58% | 96.50% |
| | G2 | 0.61% | 4.52% | 94.87% |
| Herceptin- | M3F | 0.62% | 4.95% | 94.44% |
| GlcNAc-F | G0F | 0.00% | 2.45% | 97.55% |
| | G2F | 0.21% | 2.64% | 97.14% |

M3 was able to obtain 78% fully glycosylated Herceptin at 5 minutes and deglycosylation started. To optimize the conjugation rate, increasing M3 to 30:1 (molar ratio) to attain a result of 86.3% at minute 10 before deglycosylation, and increasing to 40:1 (molar ratio) resulting in 90% of fully glycosylated Herceptin. Final condition of conjugating with M3 was 40:1 with 10 minutes reaction time to obtain 92.37% fully glycosylated Herceptin. Homogeneous platform not only can be applied on Herceptin-GlcNAc but also applied on Herceptin-GlcNAc-F to conjugating various glycans on Fc region and with results of above 90% conjugation efficiency.

### Example 12: Transglycosylation on Various Antibodies

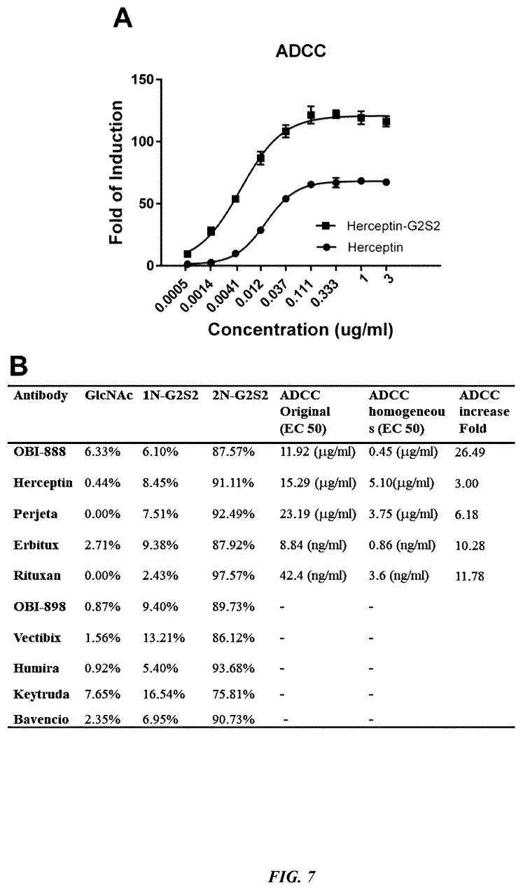

The homogeneous platform is a powerful process to establish homogeneous mAbs. Several other mAbs were selected for the conjugation investigation by EndoSz-D234M, including OBI-888, Perjeta, Erbitux, Rituxan, OBI-898, Vectibix, Humira, Keytruda, Bavencio. With the condition of 20:1 (molar ratio) of NSCT-oxa to antibody, the results demonstrated the effectiveness of homogeneous platform (FIG. 7). The percentage of fully glycosylated mAbs are OBI-888-G2S2: 87.57%, Perjeta-G2S2: 92.49%, Erbitux-G2S2: 87.92%, Rituxan-G2S2: 97.57%, OBI-898-G2S2: 89.73%, Vectibix-G2S2: 86.12%, Humira-G2S2: 93.68%, Keytruda-G2S2: 75.81% and Bavercio-G2S2: 90.73%.

To evaluate the activities of homogeneous glycan on different mAbs, we have selected five antibodies for the ADCC bioassay (FIG. 7B). The EC50 of all homogeneous mAbs were increased by comparing with original mAbs. OBI-888 increased 26 folds had the best ADCC improvement.

### Example 13: Overall Architecture of EndoSz-D234M and Complex with Glycan

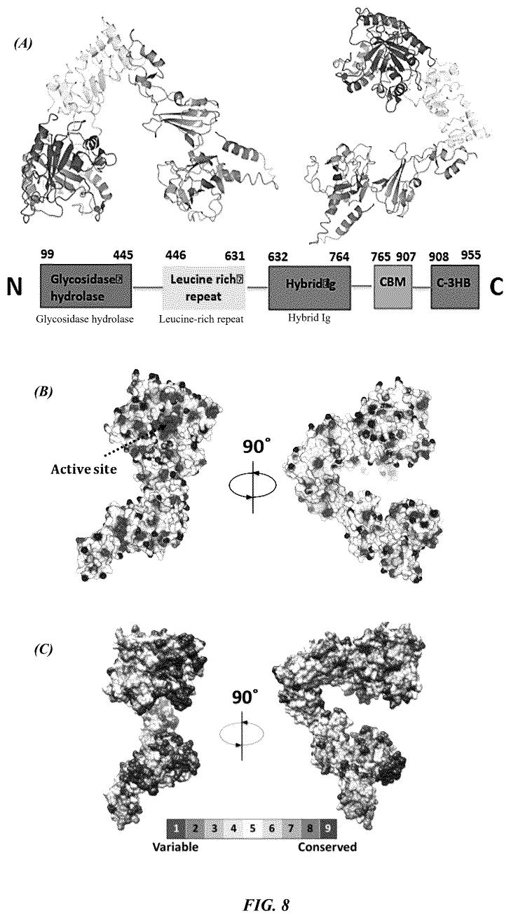

In our study, EndoSz-D234M has been identified to possess a superior transglycosylation activity on therapeutic antibodies IgGs and play a vital role on our homogenous mAbs platform. To understand the transglycosylation mechanism, we performed crystallization and structure determination of the mutant EndoSz-D234M. We designed a truncated EndoSz-D234M with amino acids 99-974 for crystallization and structural analysis. As a result, the truncation of EndoSz-D234M successfully led to acquisition of diffraction-quality crystals of EndoSz-D234M with two forms of the space groups P2₁2₁2₁ and P2₁. Crystals of P2₁2₁2₁ diffracted to a higher resolution ~2.2 Å, compared to crystals of P2₁ (~3.1 Å). The determined structures from two crystal forms showed very similar structural architectures (RMSD 0.75 Å, calculated by SSM) and no significant conformational variation between the two structures noted. Therefore, the higher resolution structure of EndoSz-D234M with space group P2₁2₁2₁ was referred throughout the manuscript. The crystal structure of EndoSz-D234M revealed a monomeric V-shaped architecture, comprising five major domains: a glycosidase hydrolase (GH) domain (a.a. 99-445), a leucine-rich repeat domain (a.a. 446-631), a hybrid Ig domain (a.a. 632-764), a carbohydrate-binding module (CBM) (a.a. 765-907), and a C-terminal 3-helix bundle domain (a.a. 908-955) (FIG. 8A). One calcium ion was coordinated in the CBM domain, of which the function is discussed in the next session. The active site of the GH domain showed a highly negatively charged surface area with conserved residues (FIG. 8B), resulting in possessing highly conserved catalytic residues and the environment for the specific Endo-β-N-acetylglucosaminidases activity (FIG. 8C). However, some loops surrounding the active site of the GH domain and the CBM domain showed more variable residues on the surface (FIG. 8C). Together with that the GH and CBM domains, located at the opposite terminus of the V-shaped architecture respectively, face to the same plane (FIG. 8A), the CBM domain might facilitate or orientate EndoSz to target N-glycans of IgG, via binding to the given location on the IgG protein or one of the two N-linked glycans for the further N-glycan cleavage or conjugation.

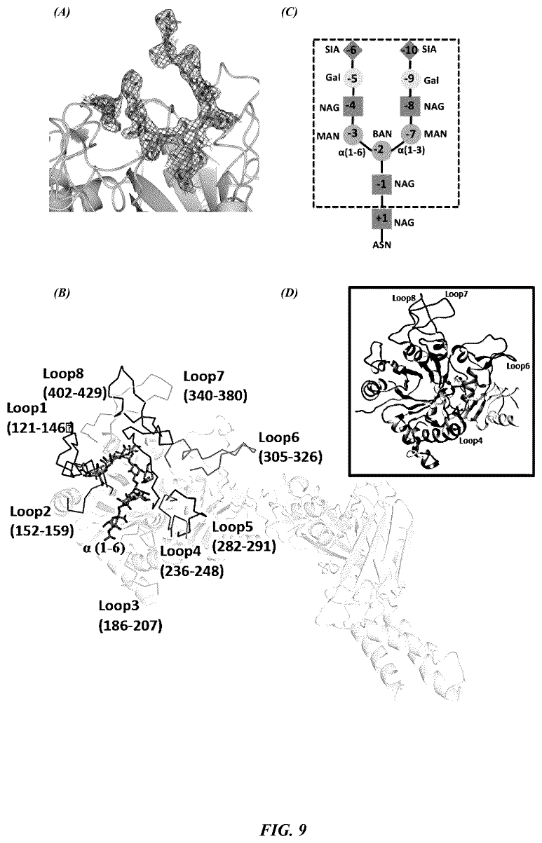

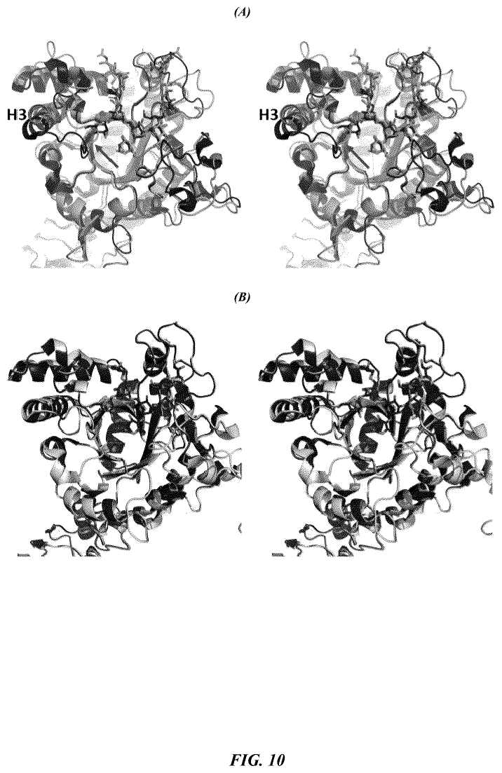

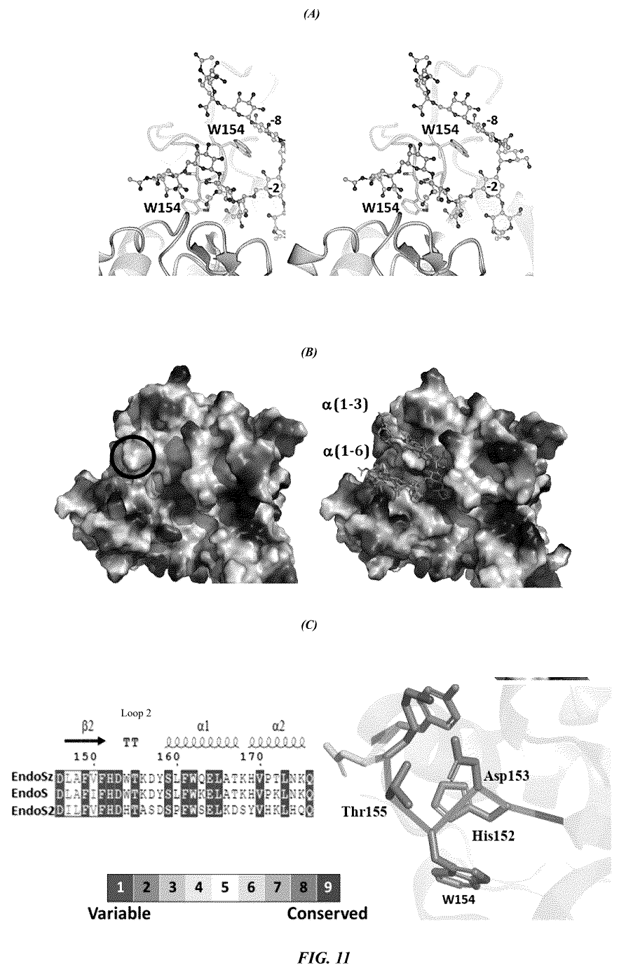

With the high-resolution data, the extra density at the GH domain was clearly identified with sufficient quality to allow us to build all 10 moieties of the CT N-glycan (FIG. 9A). As mentioned previously, the structure of the GH domain of EndoSz revealed a typical (α/β)₈ TIM barrel fold, a cyclic 8-fold repeat of β-strand/loop/α-helix composition. The bound complex-type (CT) N-glycan consisted of Manβ1-4GlcNAc disaccharides and two glycan antennas α (1-3) and α (1-6) in the (α/β)₈-barrel surrounded by the loops on the top barrel (FIG. 9B). The nomenclature of CT N-glycan utilized in the soaking experiment was shown in FIG. 9C. The two sugar moieties, Manβ1-4GlcNAc, of the CT N-glycan sat on the cavity formed by the flanked loops and the β-barrel core (FIG. 9B). As shown in FIG. 9B, the loops connecting α-helices and β-strands could be annotated loop1 (01-02; a.a. 121-146), loop2 (β2-α1; a.a. 152-159), loop3 (β3-α2; 186-207), loop4 (β4-α3; a.a. 236-248), loop5 (β5-α4; 282-291), loop6 (β6-α5; 305-326), loop7 (β9-α8; 348-380) and loop8 (β10-α9; 402-429). In addition to Manβ1-4GlcNAc disaccharides, two glycan antennas a (1-3) and α (1-6) interacted with the flanked loops, respectively. Compared to the antenna α (1-3), the antenna α (1-6) sat more closely near the cavity edge formed by the GH domain and possesses more interactions with the GH domain (FIG. 9B). The loops 3 and 4 of the GH domain interacted with the antenna α (1-6), whereas the loops 1, 2 and 7 interacted with the antenna α (1-3). As shown in FIG. 10, the varied H3 and the loop4 resulted in the inherent structure variation according to a structural comparison between EndoSz-D234M and EndoS2. The inherent structural variation indeed hindered EndoSz-D234M to bind the triantennary high-mannose triantennary N-glycan. Moreover, the conservation analysis of the flanked loops (FIG. 9D) showed that the loop4, the loop6 the loop7 and the loop8 were the more variable regions. In summary, the loop4 was suggested to play an important role in the substrate selectivity in terms of biantennary rather than triantennary glycans.

A structural comparison of apo and holo EndoSz-D234M shows that the loop2 (a.a. 152-159) exhibited the dramatic conformational variations (FIGS. 11A and B). In the apo EndoSz-D234M structure, Trp154 and Arg182 formed a typical cation-pi interaction. However, upon the structural alteration of loop2, Trp154 flipped its side chain to interact with the Man (−2) of the core disaccharides and NAG (−8) with a hydrogen bond and the weak pi stacking interaction, respectively, in the holo EndoSz-D234M structure. The dramatic movement of the loop2 reshaped the asymmetric grooves in the GH domain to accommodate two antennas of the CT N-glycans (FIG. 11B). EndoSz-D234M and EndoS both possessed the same tryptophan (Trp154 of EndoSz) in the loop2, whereas the analogue residue of EndoS2 was histidine (FIG. 11C), which could result in different substrate-binding mechanisms. In addition, the flanked residues of Trp154 (His152, Asp153 and Thr155) were highly conserved (FIG. 11C), highlighting the importance of Trp154 of EndoSz-D234M.

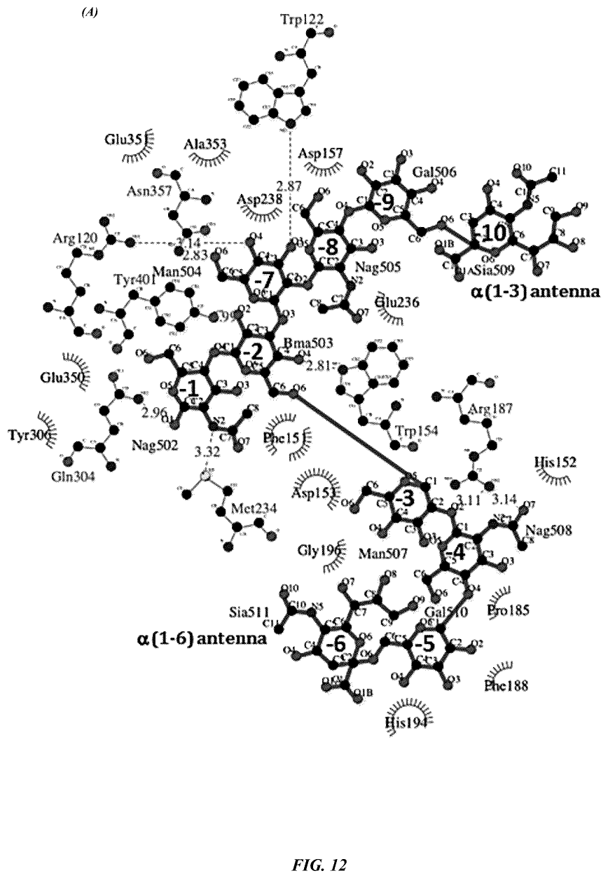

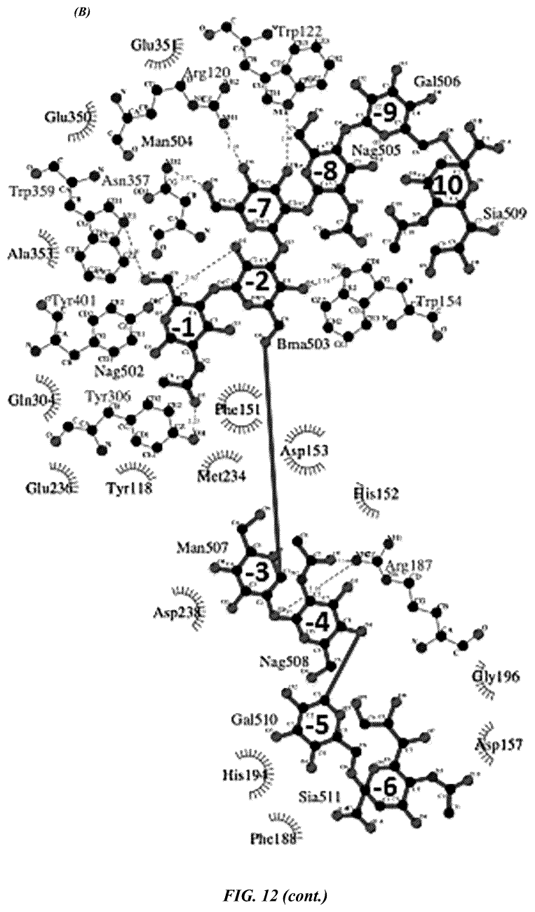

The bound CT N-glycan utilized the reducing-end GlcNAc and the trimmanose core (~2, -3 and -7) to make major contacts with EndoSz-D234M. Herein, the first GlcNAc (~1) exhibited two conformations A and B. The conformation A was the major conformation as shown in FIG. 12A. In the conformation A, the 01 atom of GlcNAc (~1) interacted with Gln304 and the O6 and O7 atoms of GlcNAc (−1) had no interaction with EndoSz. In addition, the N2 atom of the acetamido group of GlcNAc (−1) had the hydrogen bond with the side chain of Met234. In the conformation B (FIG. 12B), the 01 atom of GlcNAc (−1) had no interaction and the O6 and O7 atoms of GlcNAc (−1) interacted with Trp359 and Tyr306 respectively. The O2 and O4 atoms of Man (~2) had hydrogen bonds with the side chain of Tyr401 and the indole nitrogen of Trp154, respectively. In addition, the sugar ring of Man (−2) had the pi-stacking interaction with Phe151. The O2 atom of Man (−3) has the hydrogen bond with the side chain of Arg187. The O3, O4 and O6 atoms of the Man (−7) had hydrogen bonds with the indole nitrogen of Trp122, the side chains of Arg120 and Asn357, respectively. The major hydrogen bond interactions between the CT N-glycan and EndoSz resulted from the four moieties—GlcNAc (−1), Man (−2), Man (−3) and Man (−7) of the pentasaccharide core as our substrate in the crystallization experiment did not contain the moiety of GlcNAc (+1). In addition, GlcNAc (−8) on the antenna α (1-3) possessed the pi-pi interaction with Trp154, whereas GlcNAc (~4) on the antenna α (1-6) possessed the weak pi-pi interaction with His194 and the hydrogen bond with Arg187, which made the minor contacts between EndoSz-D234M and the bound CT N-glycan. In summary, the amino acid residues Arg 120, Trp122, Phe 151, Trp154, Arg 187, His 194, Met234, Gln304, Tyr306, Asn 357, Trp359 and Tyr401 were the binding sites of EndoSz-D234M.

### Example 14: Preparation of NSCT-2 (Oxazoline-NSCT-N3)

NSCT-1 (235 mg, 0.097 mmol; purchased from Glytech, Inc. Catalog No. GT-25261; HPLC purity >90%) and triethylamine (605 μL, 0.44 mmol) were dissolved in water (10 mL) and cooled to 0° C. 2-chloro-1,3-dimethyl-1H-benzimidazol-3-ium chloride aqueous solution (1 M, 1.44 mL) was added slowly and the resulting mixture was stirred at 0 to 5° C. for 4 hours. NaOH solution was added (0.01 M, 1 mL) and the resulting mixture was concentrated under reduced pressure. After most of the triethylamine was evaporated, the residual mixture was purified by Sephadex® g-15 column. Using 0.01M NaOH as eluent to stabilize NSCT-2. Fractions with desired product were combined and freeze-dried to afford NSCT-2 (170 mg) as a white solid. 1H NMR (D₂O): δ 6.10 (d, J=7.26 Hz, 1H, H1 of oxazoline), 5.24 (s, 1H, H1 of GluNac), 4.97 (s, 1H, H1 of GluNac), 4.76 (s, 1H, H1 of Man), 4.64-4.60 (m, 2H, H1 of two Gal), 4.46 (s, 1H, H1 of Neu5Ac), 4.45 (s, 1H, H1 of Neu5Ac), 4.40 (s, 1H, H3 of β-form Man), 4.18 (d, J=21 Hz, 4H, H2 of two Man and two GluNac), 3.99-3.48 (m), 2.71 (dd, J1=12.9 Hz, J2=4.2 Hz, 2H, H3eq of two Neu5Ac), 2.15-2.02 (m, 15H), 1.58 (dd, J1=J2=12.2 Hz, 2H, H3ax of two Neu5Ac).

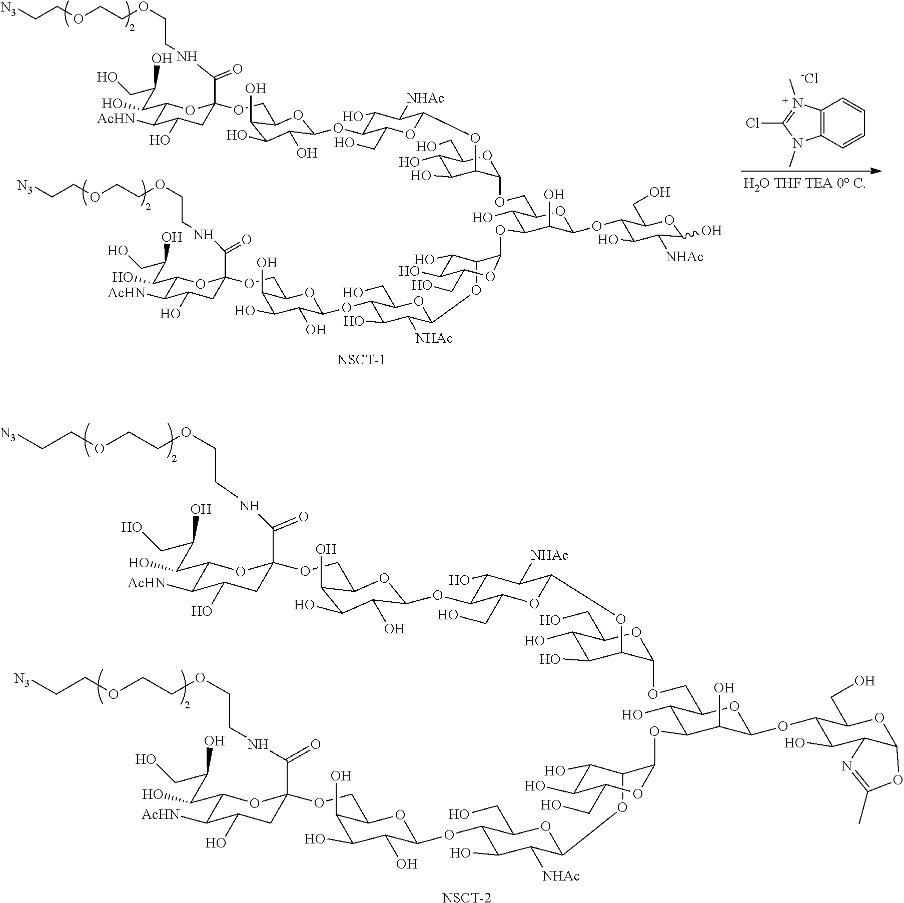

### Example 15: Deglycosylation, Transglycosylation and Purification of Monoclonal Antibodies

15-1. Deglycosylation of mAbs by EndoSz-D234M to Generate mAb-GlcNAc(Fuc)

Seven monoclonal antibodies were performed in the study. R4702 is an Anti-TROP2 monoclonal antibody and Enfortumab was internally generated and others are purchased. Herceptin (Roche), Perjeta (Roche), Erbitux (Meck), Rituxan (Roche), TX05 (Anti-HER2 mAb; Tanvex)]. The monoclonal antibodies were deglycosylated with EndoSz-D234M in 50 mM Tris pH 7.2 at 37° C. for 24-49 hours. Only the antibodies with high mannose N-glycan modification were further supplied with EndoH and incubated at 25° C. overnight to remove glycans completely and produced mAb-GlcNAc(Fuc). The complete cleavage of Fc N-glycans were analyzed by SDS-PAGE and CE-SDS.

The EndoSz-D234M for glycan ADC platform was applied to other mAbs for the deglycosylation and tranglycosylation investigation, including Herceptin, Perjeta, Rituxan, Erbitux, and Enfortumab. For the deglycosylation studies, the mAbs were incubated with EndoSz-D234M with weight ratio 1:30 (EndoSz-D234M:mAbs). Rituxan and Enfortumab were added additional EndoH to completely cleave high mannose glycans. Below results were the percentage of mAb-GlcNAc(Fuc) after deglycosylation treatment: Herceptin-GlcNAc(Fuc): 94.91%, Perjeta-GlcNAc(Fuc): 95.04%, Rituxan-GlcNAc(Fuc): 97.25%, Erbitux-GlcNAc(Fuc): 100%, and Enfortumab-GlcNAc(Fuc): 96.9%. All the mAb-GlcNAc(Fuc) reached to >90% by EndoSz-D234M cleavage. For the tranglycosylation investigation with NSCT-2, 20 or 38 equivalents of NSCT-2 was added to mAb-GlcNAc(Fuc) with EndoSz-D234M at 37° C. for 1.5 to 2 hours. The results showed that the percentage of mAb-(NSCT-di-N3)₂ were Herceptin-(NSCT-di-N3)₂: 89.27%, Perjeta-(NSCT-di-N3)₂: 94.62%, Rituxan-(NSCT-di-N3)₂: 94.88%, Erbitux-(NSCT-di-N3)₂: 93.36%, and Enfortumab-(NSCT-di-N3)₂: 95.8% by CE-SDS. These data indicate that EndoSz-D234M for glycan ADC platform can be applied to various mAbs

TABLE 7
Deglycosylation and transglycosylation of various mAbs

| | mAb | | | | | | | |
|---|---|---|---|---|---|---|---|---|
| | R4702 | | TX05 | | Herceptin | | Perjeta | |
| | mAb- | mAb- | mAb- | mAb- | mAb- | mAb- | mAb- | mAb- |
| Form | GlcNAc(Fuc) | (NSCT-di-N3)₂ | GlcNAc(Fuc) | (NSCT-di-N3)₂ | GlcNAc(Fuc) | (NSCT-di-N3)₂ | GlcNAc(Fuc) | (NSCT-di-N3)₂ |
| HC-GlcNAc | 96.42% | 2.54% | 94.5% | 1.3% | 94.91% | 8.72% | 95.04% | 3.53% |
| HC | 3.58% | 1.85% | 5.5% | 2.0% | 5.09% | 2.01% | 4.96% | 1.85% |
| HC-N3 | — | 95.61% | — | 96.7% | — | 89.27% | — | 94.62% |

| | mAb | | | | | |
|---|---|---|---|---|---|---|
| | Rituxan | | Erbitux | | Enfortumab | |
| | mAb- | mAb- | mAb- | mAb- | mAb- | mAb- |
| Form | GlcNAc(Fuc) | (NSCT-di-N3)₂ | GlcNAc(Fuc) | (NSCT-di-N3)₂ | GlcNAc(Fuc) | (NSCT-di-N3)₂ |
| HC-GlcNAc | 97.25% | 4.01% | 100.00%* | 7.34% | 96.9% | 3.3% |
| HC | 2.75% | 1.11% | — | — | 3.1% | 0.9% |
| HC-N3 | — | 94.88% | — | 93.36%* | — | 95.8% |

15-2. Transglycosylation of mAb-GlcNAc(Fuc) with Oxazoline-NSCT-N3 (NSCT-2) to Generate mAb-(NSCT-di-N3)₂

In general, mAb-GlcNAc(Fuc) were incubated with 20-38 equivalents of NSCT-2 at 37° C. for 1.5-2 hours to generate mAb-(NSCT-di-N3)₂. The transglycosylation efficiency was monitored by SDS-PAGE and CE-SDS.

15-3. Purification of mAb-(NSCT-di-N3)₂

Sodium chloride was added into the transglycosylation mixture to reach final 3M and then applied to PBS and 3M NaCl pre-equilibrated HiTrap Phenyl HP (Cytiva). The non-bound contaminations were washed by 5CV of equilibration buffer (PBS and 3M NaCl). mAb-(NSCT-di-N3)₂ was eluted with a 30-100% elution buffer (Sodium phosphate 20 mM and 20% IPA pH 7.2) in 20CV linear gradient. The eluted fractions were applied to a prepacked column, HiTrap Protein A HP (Cytiva). The impurities were washed by two steps pH gradient, 100 mM Sodium citrate pH 6.0 and pH 5.5, with 5CV in each step. 50 mM Sodium citrate pH 3.5 was employed to elute bound antibody. The eluted fractions were immediately neutralized with 1 M Tris-HCl pH 9.0 to natural pH and change buffer to 20 mM Sodium Acetate pH 5.0 with Amicon centrifugation membrane (30 kDa cutoff, Millipore). The purified mAb-(NSCT-di-N3)₂ were stored at −80° C.

### Example 16: ADC Preparation

### 16-1. MCCA-PEG₂₄-VA-PAB-Exatecan (Linker-Drug Compound 1) Preparation

### Step 1:

To a suspension of exatecan mesylate in DMF, N-PM-0015 and DIPEA were added at room temperature. The suspension became a clear brown solution within 5 minutes. This mixture was stirred at room temperature for 20 hours. After the reaction was completed, the reaction mixture was added to a stirring TBME over 30 minutes to get precipitate. After stirring for 30 minutes, the solids were collected by filtration and followed with high vacuum drying to obtain crude N-PM-0016. This crude product was used in the next step without further purification.

### Step 2:

A stirring suspension of N-PM-0016 in DCM was cooled to −20° C. A −10° C. pre-cooled TFA liquid was added to N-PM-0016 solution over 60 minutes. This mixture was stirred at −20° C. for 10 hours. After the reaction was completed, the reaction mixture was added to a stirring TBME over 30 minutes to get precipitate. After stirring for 30 minutes, the solids were collected by filtration and followed with high vacuum drying to obtain crude N-PM-0018. This crude product was used in the next step without further purification.

### Step 3:

To a solution of N-DT-0013 in DMF, HATU and NMM was added. This mixture was stirred at room temperature for 2 hours. A solution of N-PM-0018 and NMM in DMF was added to the N-DT-0013 solution at room temperature for over 30 minutes. This mixture was stirred at room temperature for a further 2 hours. After the reaction was completed, the reaction mixture was added to a stirring TBME over 30 minutes to get precipitate. After stirring for 30 minutes, the solids were collected by filtration and purified by reverse phase chromatography (eluent: ACN/Water). The pure fractions were combined and extracted with 10% MeOH/DCM to obtain N-PM-0017.

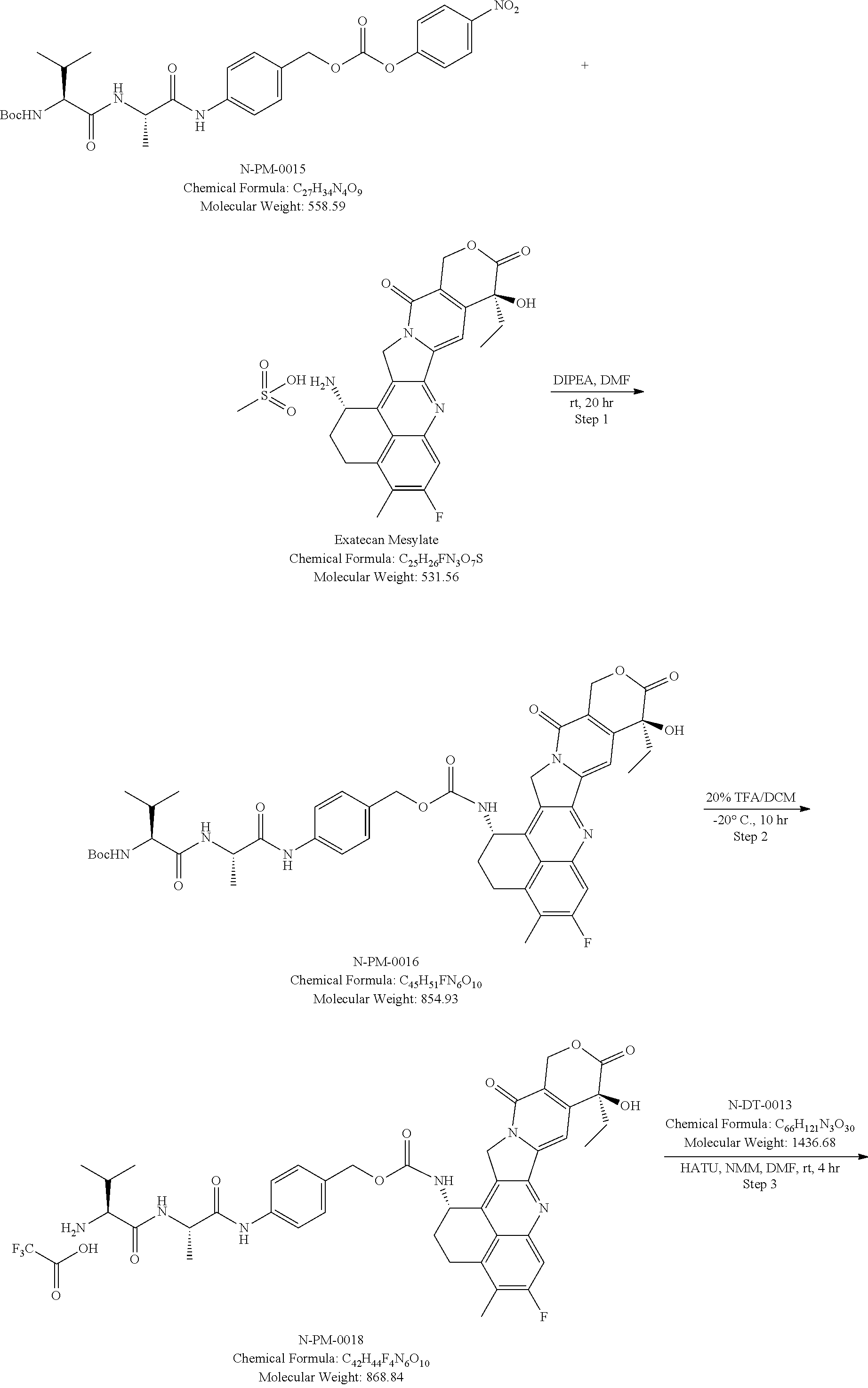

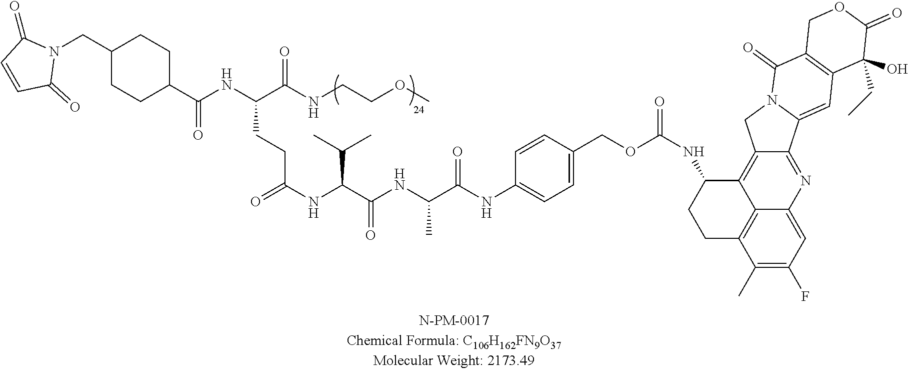

### 16-2. DBCO-PEG₂₄-VA-PAB-Exatecan (Linker-Drug Compound 2) Preparation

### Step 1:

N-(9-Fmoc)-L-glutamic acid γ-tert-butyl ester monohydrate (152.2 mg, 0.35 mmol), m-PEG24-amine (380.9 mg, 0.35 mmol) and HATU (159.7 mg, 0.42 mmol) were dissolved in DMF/CH₂Cl₂=1/1 (3.5 mL) under room temperature. NMM (115.8 μL, 1.05 mmol) was added. After the addition, the resulting mixture was stirred at room temperature for 18 hours. The reaction solution was concentrated in vacuo at 30-35° C., the residue was purified by flash silica gel column (CH₂Cl₂/MeOH=15/1 to 12/1) to obtain 502.5 mg of Glu-1 with 96.0% yield.

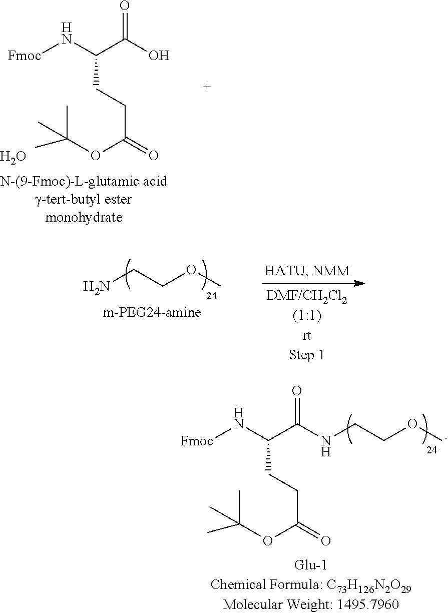

### Step 2:

To a solution of Glu-1 (413.8 mg, 0.27 mmol) in CH₂Cl₂/MeOH=1/1 (13.8 ml), Et₂NH (1.38 ml) was added. The mixture was stirred at room temperature for 24 hours. After reaction was completed, the reaction mixture was concentrated in the vacuo at 30-35° C. and then azeotroped with Toluene (5 mL×3) to remove excess Et₂NH. The resulting material was dried with high vacuum to obtain crude Glu-2. The crude product was used in next step without further purification.

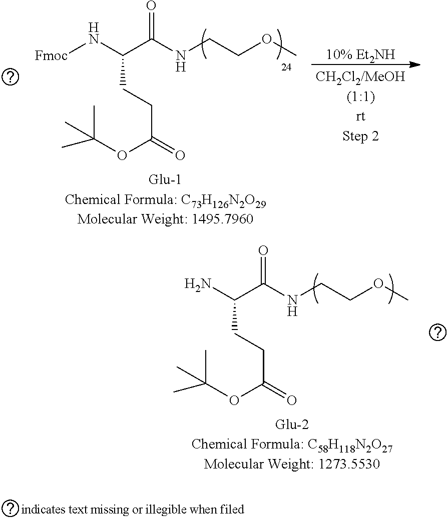

### Step 3:

To a solution of crude Glu-2 (352.3 mg, 0.27 mmol) and DBCO-acid (101.4 mg, 0.33 mmol) in DMF/CH₂Cl₂=1/1 (5.5 ml), HATU (157.8 mg, 0.45 mmol) and NMM (91.6 μL, 0.83 mmol) were added separately. The reaction mixture was stirred at room temperature for 16 hours. After reaction was completed, the reaction solution was concentrated in the vacuo at 30-35° C., the residue was purified with flash silica gel column (CH₂Cl₂/MeOH=15/1) to obtain 367.9 mg of DBCO-1 with 85.2% yield from Glu-1.

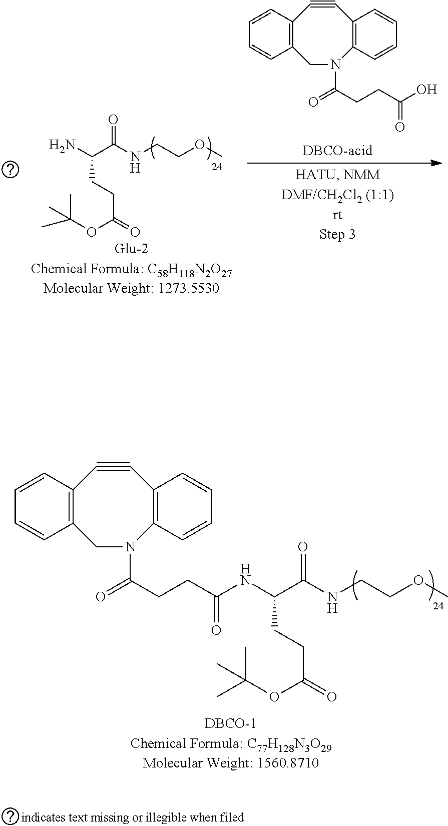

### Step 4:

To a solution of DBCO-1 (350.0 mg, 0.22 mmol) in CH₂Cl₂ (6.9 mL) was cooled to 0° C. TFA (1.8 mL) was added dropwise. The reaction mixture was stirred at 0° C. for 4-6 hours. After reaction was completed, the reaction mixture was concentrated in the vacuo at 30-35° C., the residue was purified with flash silica gel column (CH₂Cl₂/MeOH=12/1) to obtain 175.4 mg of DBCO-2 with 52.0% yield.

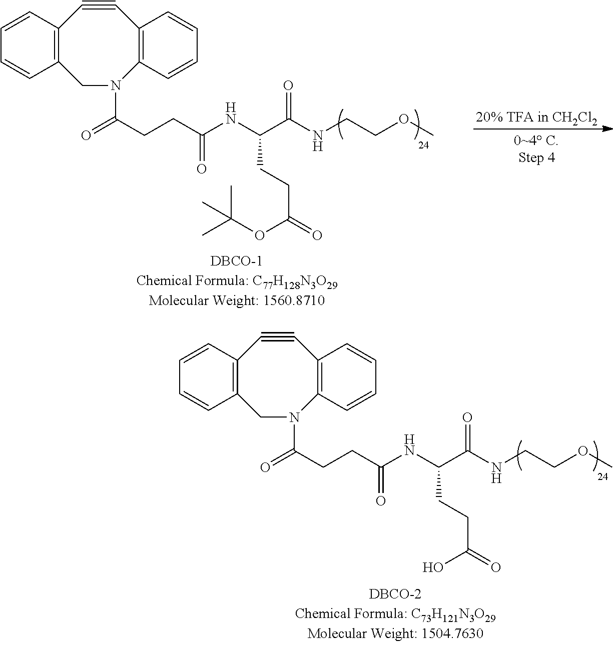

### Step 5:

N-PM-0018 (26.5 mg, 0.031 mmol), DBCO-2 (45.9 mg, 0.031 mmol) and HATU (13.9 mg, 0.037 mmol) were dissolved in DMF (0.61 mL). NMM (10.1 μL, 0.092 mmol) was added. The reaction mixture was stirred at room temperature for 20 hours. After reaction was completed, the resulting mixture was purified with preparative HPLC to obtain 48.9 mg of DL-2 with 71.5% yield. ¹H NMR (600 MHz, d-MeOH) δ 7.70-7.50 (m, 6H), 7.46-7.36 (m, 5H), 7.33-7.10 (m, 3H), 5.56 (d, 1H, J=16.0 Hz), 5.36 (dd, 1H, J=16.0, 6.2 Hz), 5.33-5.29 (m, 1H), 5.27 (d, 1H, J=20.2 Hz), 5.21-5.13 (m, 2H), 5.13-5.05 (m, 2H), 4.47-4.41 (m, 1H), 4.23-4.12 (m, 2H), 3.73-3.52 (m, 102H), 3.50-3.46 (m, 1H), 3.43-3.38 (m, 1H), 3.35 (s, 3H), 3.27-3.17 (m, 1H), 3.15-3.07 (m, 1H), 2.81-2.70 (m, 1H), 2.37 (s, 3H), 2.36-2.24 (m, 4H), 2.45-2.40 (m, 1H), 2.24-2.00 (m, 5H), 2.00-1.78 (m, 4H), 1.43 (d, 1H, J=7.1 Hz), 1.03-0.94 (m, 9H); HRMS (ESI) m/z found [(M+2H)/2]⁺, 1121.0605 C₁₁₃H₁₆₄FN₉O₃₆²⁺, required 1121.0553.

TABLE 8
HPLC condition (Column: YMC-Actus Trait C18
250 × 20 mm, 5 μL, 12 nm)
Gradient:

| | | | |
|---|---|---|---|
| Time | Flow (mL/minute) | H₂O | ACN |

| | | | | |
|---|---|---|---|---|
| 0 | min | 10 | 90% | 10% |
| 1 | min | 20 | 70% | 30% |
| 5 | min | 20 | 55% | 45% |
| 20 | min | 20 | 45% | 55% |
| 21 | min | 20 | 10% | 90% |
| 30 | min | 20 | 10% | 90% |

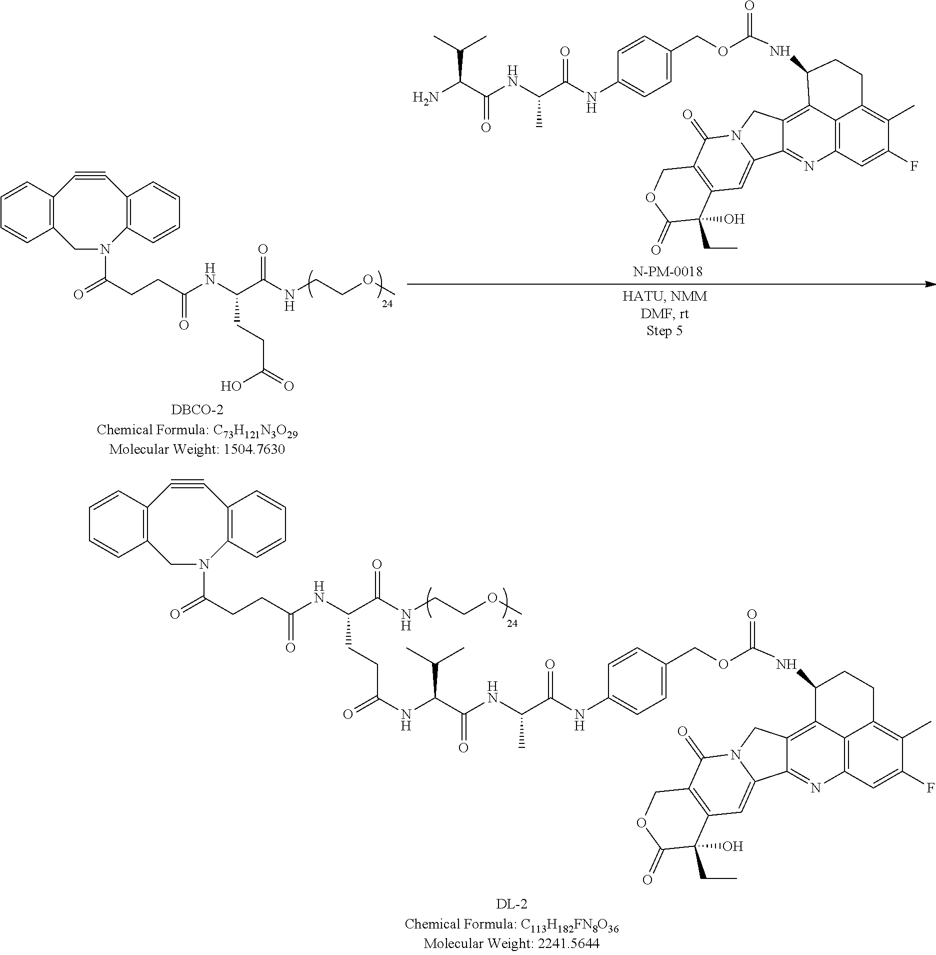

### 16-3. ADC Preparation by Using MCCA-PEG24-VA-PAB-Exatecan (Linker-Drug Compound 1)

R4702 and TX05 mAbs (10 mg/mL, total 50 mL) within reaction buffer (50 mM Histidine, 20 mM EDTA, pH 7.0) was cooled to 12-16° C. R4702 and TX05 were treated with TCEP-HCl (2.29 mg; 0.00799 mmol) in reaction buffer (0.46 mL) for 2-6 hours at 12-16° C. In order to reduce the antibody solution, payload-linker N-PM-0017 (39.94 mg; 0.0183 mmol) within DMSO was added and conjugated for one hour at 12-16° C. After conjugation completed, the buffer was changed to the storage buffer (20 mM Sodium acetate, pH 5.0 with 0.1% (w/w) polysorbate 80) via UF/DF dialysis membrane to achieve final concentration of 10.14 mg/mL and total 41.9 mL. The average drug-to-antibody ratio (DAR) value of final R4702-MCCA-ADC (ADC-1) and TX05-MCCA-ADC (ADC-3) is 4.5 and 4.7 determined by hydrophilic interaction chromatography (HIC).

### 16-4. ADC Preparation by Using DBCO-PEG₂₄-VA-PAB-Exatecan (Linker-Drug Compound 2)

DL-2 (16.35 mg) was dissolved in 1635 μL DMSO to form a DL-2 solution. The DL-2 solution (1614 μL) was added slowly to R4702-(NSCT-diN3)₂ solution (18 mL, antibody concentration 5 mg/mL in 20 mM NaOAc, pH 5.0) and shook at 25° C. for 6.5 hours. After the conjugation was completed, the residual DL-2 was partially removed by buffer exchange (20 mM NaOAc, pH 5.0) using PES Amicons. The crude ADC was further purified by HIC column to afford ADC-2. After exchanging the buffer to storage buffer (20 mM NaOAc, pH 5.0), the concentration of R4702-DBCO-ADC (ADC-2) was adjusted to 5.02 mg/mL and sterilized by passing through ProMax™ Syringe Filter (PVDF, 0.22 μm). Finally, 9.9 mL of ADC-2 was produced with drug-to-antibody ratio (DAR) value 3.8 (determined by HIC).

Furthermore, DL-2 (34.58 mg) was dissolved in 1729 μL DMSO to form a DL-2 solution. The DL-2 solution (1108 μL) was added slowly to TX05-(NSCT-diN3)₂ solution (25.33 mL, antibody concentration 4.88 mg/mL in 20 mM NaOAc, pH 5.0) and stirred at 25° C. for 6 hours. After the conjugation was completed, the crude ADC was further purified by using Spectrum® Hollow Fiber Filter Modules (buffer: 20 mM NaOAc, pH 5.0) to afford TX05-DBCO-ADC (ADC-4). ADC-4 was adjusted to around 5 mg/mL and sterilized by passing through ProMax™ Syringe Filter (PVDF, 0.22 μm). Finally, 17.7 mL of ADC-4 (concentration: 4.53 mg/mL) was produced with drug-to-antibody ratio (DAR) value 3.9 (determined by HIC).

### Example 17: Glycan Engineering ADC Analysis

### 17-1. CE-SDS Analysis for Deglycosylation and Glycosylation Determination

The CE-SDS analysis was conducted under reducing condition. Beckman Coulter PA800Plus system equipped with a UV photodiode array detector (220 nm wavelength employed) was used in this test. A bare fused-silica capillary (50 m ID×30 cm total length) with the 20 cm effective capillary separation length was rinsed with 0.1 M NaOH, 0.1 M HCl and SDS gel buffer prior to injection. Electrokinetic injection mode was applied at −5 kV for 20 s in reverse polarity and followed by applying a −15 kV voltage for capillary separation. The total separation time was 35 minutes. 60 μg of Test article was sampled and diluted in 120 μL sample buffer which was 1% SDS in diluted PBS, pH 7.0. Then diluted sample was mixed with 5 μL of 2ME, and 2 μL of 10 kDa internal standard, followed by incubation at 65° C. for 10 min. Finally, the sample was cooled down at room temperature for CE-SDS analysis.

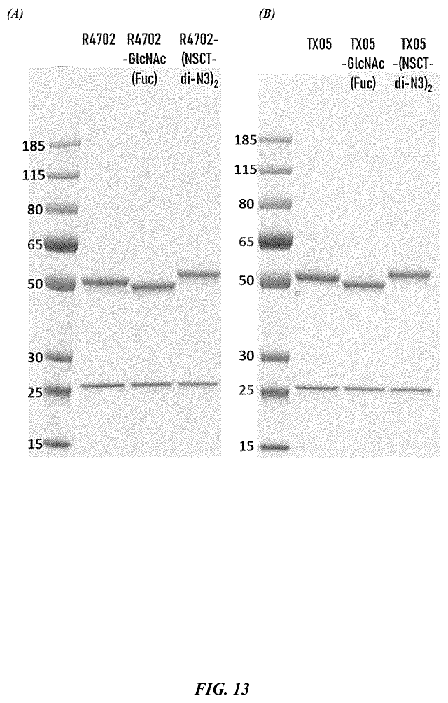

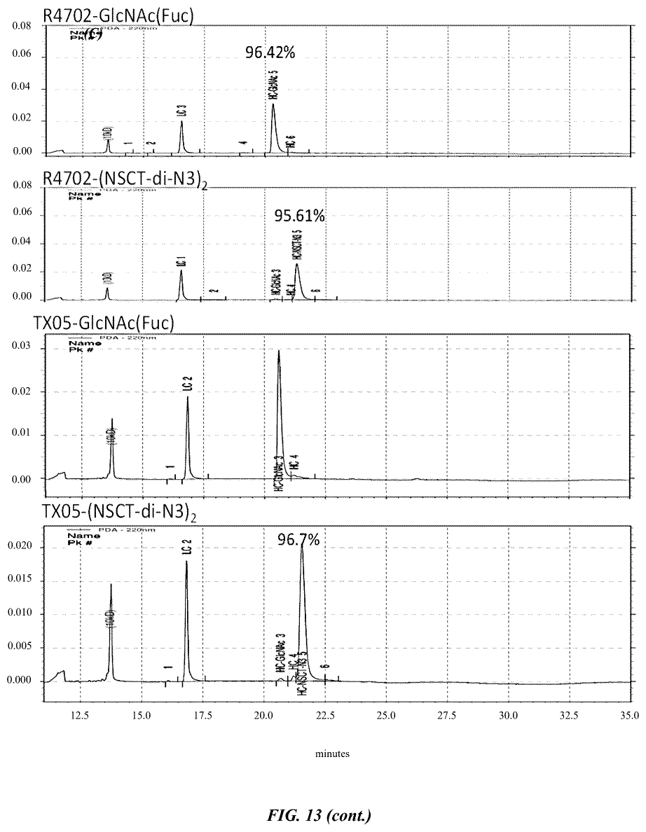

EndoSz-D234M showed high deglycosylation and transglycosylation activity for glycan ADC platform. We used R4702 and TX05 mAbs to produce R4702-DBCO-ADC (ADC-2) and TX05-DBCO-ADC (ADC-4) by EndoSz-D234M as a model of glycan ADC production. For ADC-2, R4702 mAb were deglycosylated by EndoSz-D234M with additional enzyme EndoH to cleave high mannose glycans. The yield of deglycosylated R4702 with one GlcNAc or potential fucose (R4702-GlcNAc(Fuc)) was about 96.42%. Next, R4702-GlcNAc(Fuc) were mixed with 20 equivalents of a modified complex type N-glycan (NSCT-2) at 37° C. for 1.5 hr to generate R4702-(NSCT-di-N3)₂. The CE-SDS results indicated that 96.51% R4702-(NSCT-di-N3)₂ was produced by EndoSz-D234M. For ADC-4, EndoSz-D234M was used to hydrolyze biantennary hybrid glycan on TX05 mAb and generate 94.5% TX05-GlcNAc(Fuc). In the tranglycosylation step, NSCT-2 were added to TX05-GlcNAc(Fuc) and EndoSz-D234M mixture. With 15 equivalents of NSCT-2 to TX05-GlcNAc(Fuc), 96.7% of TX05-(NSCT-di-N3)₂ obtained by EndoSz-D234M catalysis at 15° C. for 4.5 hr incubation (FIG. 13C). These data demonstrate that EndoSz-D234M effectively generates mAb-(NSCT-di-N3)₂ for glycan ADC production.

### 17-2. Intact MW Analysis by LC-MS

Intact MW analysis was conducted by Q-Exactive mass spectrometer (Thermo Scientific) coupled with vanquish HPLC system (Thermo). The LC separation was performed using Agilent PLRP-S column with the gradient program. The test sample was diluted to 0.5 mg/mL with H2O and no further deglycosylation or reduction process before LC-MS analysis. Full MS scans were performed with the ranges of m/z 1500-5000 for molecular weight analyses. Protein Deconvolution 4.0 was used to process the raw data, obtain the molecular weights.

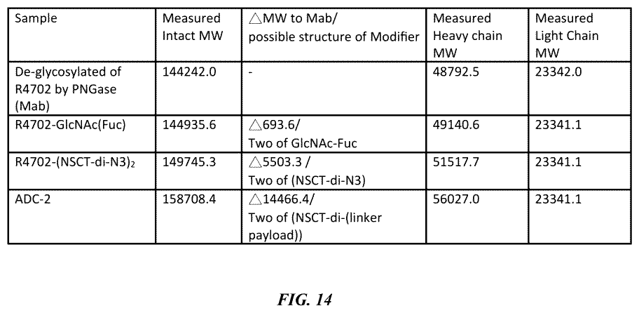

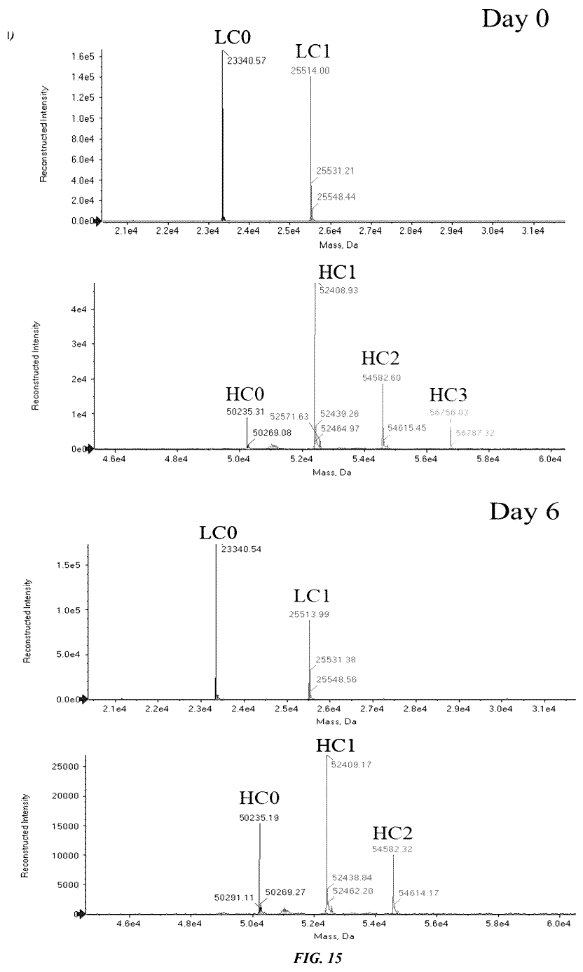

The results of intact molecular weights analysis and reduced MS analysis for each species are shown in FIG. 14. It demonstrates the deglycosylation and transglycosylation by EnddoSz-D234M. The mass difference between R4702-GlcNAc(Fuc) and R4702 indicated the N-Glycan structure on R4702-GlcNAc(Fuc) was GlcNAc(Fuc). The N-glycan of R4702 could be cleaved and formed R4702-GlcNAc(Fuc) which was evaluated by the intact MW analysis. The MW of R4702-(NSCT-di-N3)2 illustrated NSCT-2 was successfully transferred to R4702-GlcNAc(Fuc) by EndoSz-D234M. The MW of R4702-DBCO-ADC (ADC-2) demonstrated there were four payloads conjugated with R4702. Furthermore, the results of reduced-MW analysis also demonstrated that the modification was located on heavy chain.

### 17-3. DAR (Drug Antibody Ratio) Analysis in the Presence of Human Serum Albumin (HSA)

Maleimide linkers have been used extensively for conjugation of antibody and payloads. Nevertheless, the thioether connection undergoes deconjugation via a retro-Michael reaction, resulting in payload loss and lowered efficacy. Subsequently, the maleimide-linked payload can attach to plasma thiols (such as human serum albumin, HSA), causing off target toxicity.1,2 To comparison the DAR change in the present of HSA, R4702-MCCA-ADC (ADC-1) and R4702-DBCO-ADC (ADC-2) were added to 3% HSA in PBS to a final concentration of 500 μg/mL and incubated at 37° C. for 0, 24, 144 hours in a shaking water bath. The incubated samples were purified with anti-idiotype antibody coated streptavidin magnetic beads. The mixture was gently shaken for 1.5 hour at room temperature then washed three times with HEPES buffered salt solution, two times with deionized water and eluted with 2% formic acid. For DAR change calculation, the eluted ADCs were reduced with TCEP (final concentration, 20 mM) at room temperature for 30 minutes to form light chain (LC) and heavy chain (HC) fragments and followed by LC-HRMS analysis. The following equation was used for average DAR calculation:

$$( L C 1 / ( L C 0 + L C 1 ) ) \times 2 + ( H C 1 / ( H C 0 + H C 1 + H C 2 + H C 3 ) ) \times 2 + ( H C 2 / ( H C 0 + H C 1 + H C 2 + H C 3 ) ) \times 4 + HC 3 / ( H C 0 + H C 1 + H C 2 + H C 3 ) ) \times 6 .$$

where LC0 and LC1 is the reconstruction area of the signal for the light chain with zero and one payloads, respectively, and HC0, HC1, HC2, and HC3 is the reconstruction area of the signal for the heavy chain with 0, 1, 2, 3 payloads, respectively.

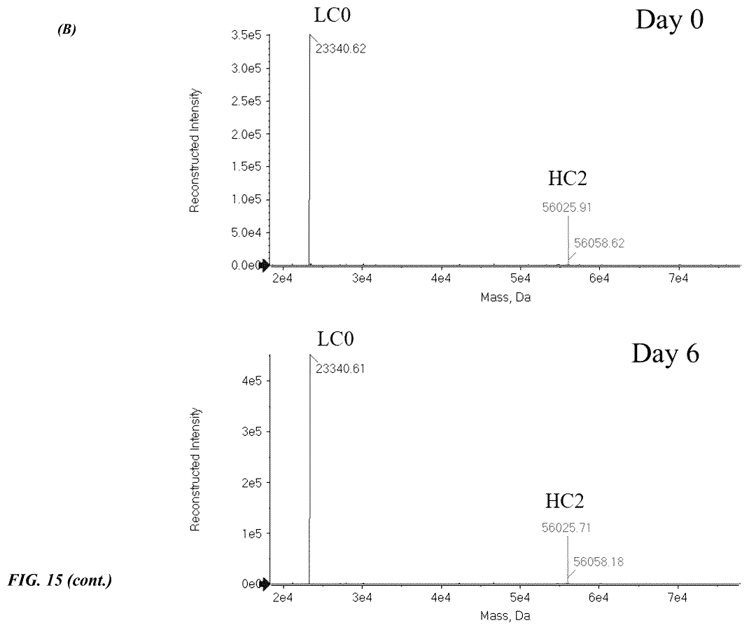

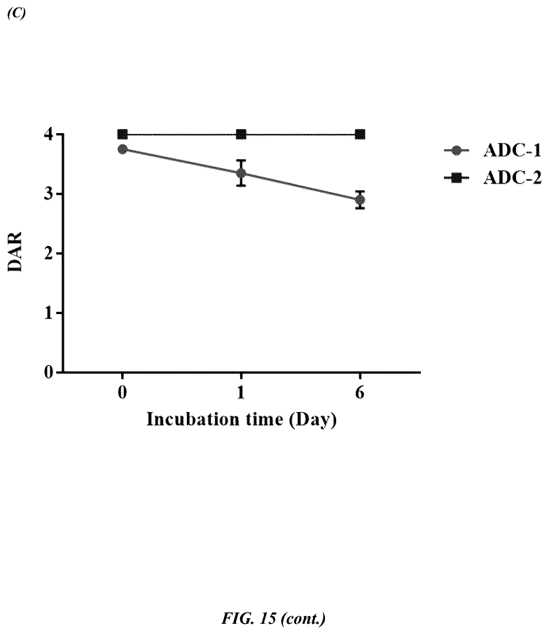

The deconvoluted mass spectrum of ADC-1 and ADC-2 which incubated in HSA for 0, 144 hours were shown in FIG. 15. After 144 hours (six days) incubation, the maleimide linker-payload loss was observed of ADC-1 (FIG. 15A). However, no extra peaks corresponding to HC2 degradation products were detected of ADC-2 (FIG. 15B). According to the formula of DAR, a time-dependent decrease in the average DAR was observed of ADC-1 (FIG. 15C). The result indicated ADC-2 didn't undergo deconjugation via a retro-Michael reaction.

### 17-4. Measurement of In-Vitro Human Plasma Stability

R4702-MCCA-ADC (ADC-1) and R4702-DBCO-ADC (ADC-2) were added to the pooled human plasma (prepared by heparin) to a final concentration of 200 μg/mL and incubated at 37° C. for 0, 24, 96, 168, 336 hours in a shaking water bath. For determination of released payload, the incubated samples were deproteinized using acetonitrile (ACN) with internal standard (IS) and followed by LC-MS/MS analysis. The percentage of theoretical maximum is calculated by released payload amount/theoretical amount of ADC DAR4 payload×100%.

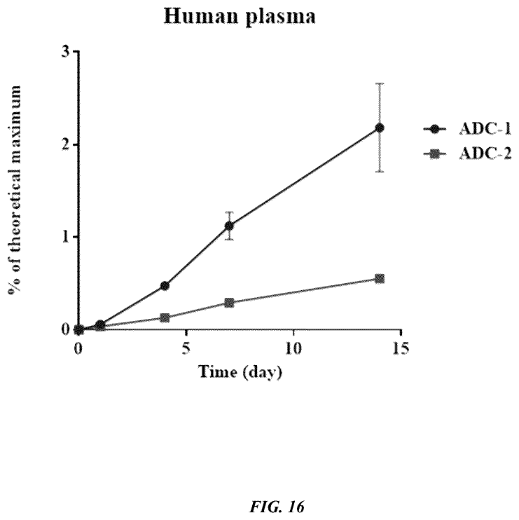

The stability of ADC-1 and ADC-2 in human plasma was monitored over 14 days at 37° C. Based on the proportion of released payload in theoretical amount of payload (DAR 4), the released payload percentage increased over the time of ADC-1 and ADC-2 as shown in FIG. 16. After 14 days of incubation in human plasma, the released payload percentage of ADC-1 and ADC-2 were around 2.2% and 0.6%. This result indicated that the ADC-2 released less payload than ADC-1 in human plasma.

### Example 18: In-Vitro Cytotoxicity Assay and of the Glycan Engineered ADCs

The Tumor cells (2×10³ cells/well) were seeded in 96 well plate and treated with ADCs for 6 days. CellTiter-Glo® Reagent (Cat. G7572, Promega) was prepared by adding CellTiter-Glo® Buffer into lyophilized CellTiter-Glo® Substrate. Reconstituted CellTiter-Glo® Reagent was added into the culture medium with cells at 1:1 ratio after treatment for 6 days. The plate was placed on an orbital shaker for 2 minutes to induce cell lysis and then incubated at room temperature for 10 minutes before recording the luminescent signals by Luminometer. The viability of each treated sample was compared to non-treated control. The IC₅₀ of each ADC was calculated by Prism.

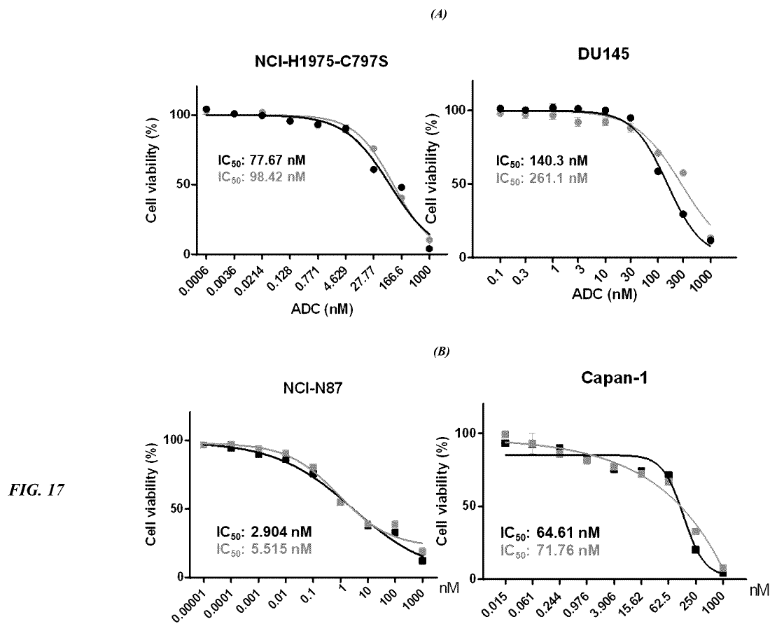

The in vitro efficacies of ADCs in several tumor cell lines were evaluated by cytotoxicity assay (FIG. 17). The IC₅₀ of R4702-MCCA-ADC (ADC-1) and R4702-DBCO-ADC (ADC-2) in NCI-H1975-C797S lung cancer cells were similar (77.64 and 98.42 nM). In DU145 prostate cancer cells, the IC₅₀ of ADC-1 was slightly lower than ADC-2 (140.3 and 261.1 nM). Similar results were observed in TX05-MCCA-ADC (ADC-3) and TX05-DBCO-ADC (ADC-4). The IC₅₀ of ADC-3 was slightly lower than ADC-4 in NCI-N87 gastric cells (2.904 and 5.515 nM), while ADC-3 and ADC-4 showed similar IC₅₀ in Capan-1 pancreatic cancer cells (64.61 and 71.76 nM). These data suggest that site-specific glycan conjugated ADCs (ADC-2 and ADC-4) exhibited similar or slightly reduced cytotoxicity than cysteine conjugated ADCs (ADC-1 and ADC-3).

### Example 19: In-Vivo Efficacy Study in NCI-H1975-C797S Lung Carcinoma Cell-Derived Xenograft of the Glycan Engineered ADCs

NCI-H1975-C797 human lung cancer cells were used to evaluate the in vivo efficacy of R4702-MCCA-ADC (ADC-1) and R4702-DBCO-ADC (ADC-2). Female BALB/c nude mice were housed in specific pathogen-free condition. Mice were acclimated for at least 3 days before the study initiation. The food (LabDiet 5010, PMI, USA) and water (sterile RO water) were provided ad libitum throughout the whole study period. All animal studies were approved by the Institutional Animal Care and Use Committee at National Laboratory Animal Center in Taiwan.

Tumor cells were washed and re-suspend in PBS. Viable cells (1×10⁷ cells/mouse) were mixed with the same volume of Matrigel (Cat. 356234, BD) and subcutaneously injected into right flank of female BALB/c nude mice (200 μL/mouse). Tumor-bearing mice were divided into distinct groups when the average tumor volume reached 150-200 mm³. ADCs or vehicle control were treated as a single dose thorough tail vein injection. The day of administration was denoted as Day 1. Tumor growth and mouse body weight were monitored twice weekly until Day 22. The efficacy of ADCs were evaluated as Tumor Growth Inhibition (TGI). The TGI was calculated by the following formula: TGI (%)=[1−(Ti−T1)/(Ci−C1)]×100%. Ti and Ci indicate the mean tumor volume in the treatment groups and vehicle group at the end of the study (Day 22), while T1 and C1 indicate the mean tumor volumes in the treatment group and vehicle group at the beginning of test item administration. The experimental design, test articles, dose concentrations, dosing frequencies, route of administration and animal numbers are listed in Table 9.

TABLE 9
Dosing regimen and sampling

| | Route and | Dose of | Volume of | |
|---|---|---|---|---|
| | administration | injection | injection | Animal |
| Groups | of test item | (mg/kg) | (mL/kg) | Number |
| Vehicle | IV | — | 5 | 5 |
| ADC-1 (10 mg/kg) | IV | 10 | 5 | 5 |
| ADC-2 (10 mg/kg) | IV | 10 | 5 | 5 |
| ADC-1 (3 mg/kg) | IV | 3 | 5 | 5 |
| ADC-2 (3 mg/kg) | IV | 3 | 5 | 5 |

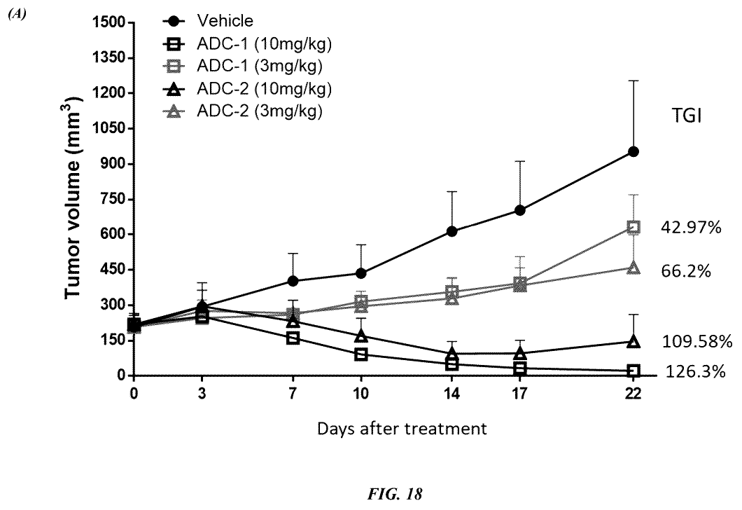

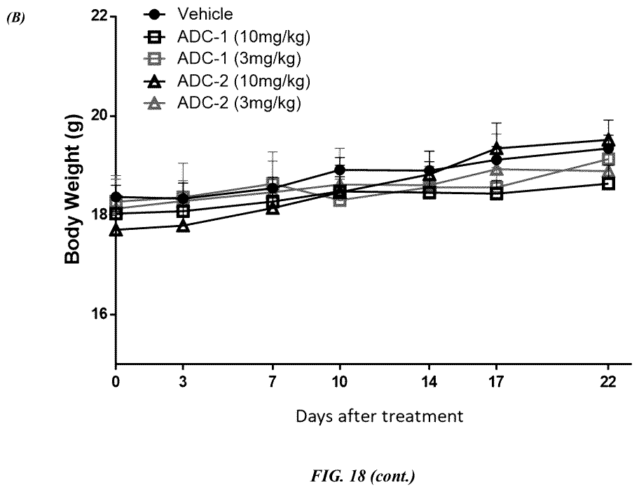

The in vivo efficacy of ADC-1 and ADC-2 were evaluated by NCI-H1975-C797S lung cancer xenograft mouse model. NCI-H1975-C797S cancer cells were implanted into BALB/c nude mice. The tumor growth (FIG. 18A) and mouse body weight (FIG. 18B) were recorded throughout the study. The tumor growth inhibition (TGI) was used to evaluate antitumor efficacy. TGI was calculated by comparing treated groups to vehicle control based on the tumor size on Day 1 and 22. Both ADC-1 and ADC-2 treated at 10 mg/kg exhibited similar excellent antitumor efficacies (TGI>100%, P=0.24). At 3 mg/kg, ADC-1 and ADC-2 showed similar partial inhibition of tumor growth (TGI: 42.7% and 66.2%, respectively, P=0.37). The results suggest that site-specific glycan conjugated ADC-2 exhibited similar antitumor efficacy as cysteine conjugated ADC-1.

The present disclosure discloses selected glycosynthase variants that show excellent transglycosylation activities with a broad range of N-glycans, including high mannose, hybrid, and complex types.

In preferred embodiments, N-glycans of high mannose, hybrid and complex types are in an active oxazoline form.

In some embodiments, the high mannose type N-glycans described herein are selected from group consisting of Man₃GlcNAc, Man₅GlcNAc, Man₆GlcNAc, Man₇GlcNAc, Man₅GlcNAc, and Man₉GlcNAc. In preferred embodiments, the high mannose type N-glycan is Man₅GlcNAc.

In some embodiments, the hybrid type N-glycans described herein comprise at least one α-2,6- or α-2,3 terminal sialic acid on the alpha-1,3 arm, wherein the alpha-1,6 arm contains the trimannose residues.

In some embodiments, the hybrid type N-glycans described herein comprise at least one terminal galactose on the alpha-1,3 arm, wherein the alpha-1,6 arm contains the trimannose residues.

In some embodiments, the hybrid type N-glycans described herein comprise at least one terminal GlcNAc on the alpha-1,3 arm, wherein the alpha-1,6 arm contains the trimannose residues.

In some embodiments, the complex type glycans are of bi-, tri- and tetra-antennary complex types.

In some embodiments, the bi-antennary complex type N-glycans described herein comprise at least one α-2,6 or α-2,3 terminal sialic acid. In preferred embodiments, the N-glycans comprise two α-2,6 and/or α-2,3 terminal sialic acids.

In some embodiments, the bi-antennary complex type N-glycans described herein comprise at least one terminal galactose or GlcNAc. In preferred embodiments, the N-glycans comprise two terminal galactose and/or GlcNAc.

In some embodiments, the bi-antennary complex type N-glycans described herein comprise at least one alpha-1,2-fucose. In preferred embodiments, the N-glycans comprise two alpha-1,2-fucoses.

In some embodiments, the bi-antennary complex type N-glycans described herein comprise at least one alpha-1,3-fucose. In preferred embodiments, the N-glycans comprise two alpha-1,3-fucose.

In some embodiments, the bi-antennary complex type N-glycans described herein comprise bisecting GlcNAc.

In some embodiments, the bi-antennary complex type N-glycans described herein comprise at least one LacNAc repeat unit. In preferred embodiments, the N-glycans comprise two LacNAc repeat units.

In some embodiments, the tri-antennary complex type N-glycans described herein comprise at least one α-2,6 or α-2,3 terminal sialic acid. In preferred embodiments, the N-glycans comprise three α-2-6 and/or α-2,3 terminal sialic acids.

In some embodiments, the tri-antennary complex type N-glycans described herein comprise at least one terminal galactose or GlcNAc. In preferred embodiments, the N-glycans comprise three terminal galactose and/or GlcNAc.

In some embodiments, the complex type glycans are of bi-, and triantennary complex types comprising asymmetric antennae on either the alpha-1,3 or alpha-1,6 arm.

In some embodiments, the hybrid and bi-, and triantennary complex type N-glycans described herein comprise α-2,6 or α-2,3 terminal sialic acid. In other embodiments, the hybrid and bi-, and triantennary complex type N-glycan comprises α-2,6 terminal sialic acid.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

Unless defined otherwise, all technical and scientific terms used herein have the same meanings as commonly understood by one of ordinary skills in the art to which this invention belongs. Although any methods and materials similar or equivalent to those described herein can be used in the practice or testing of the present disclosure, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Before the present materials and methods are described, it is understood that this invention is not limited to the particular methodology, protocols, materials, and reagents described, as these may vary. It is also to be understood that the terminology used herein is for the purpose of describing particular embodiments only and is not intended to limit the scope of the present disclosure, which will be limited only by the appended claims.

### ADDITIONAL EMBODIMENTS

Embodiment 1: A method for preparing an engineered bioconjugate, comprising contacting a biomolecule with a glycosynthase and a modified glycan thereby obtaining a first engineered bioconjugate, wherein the biomolecule further comprises a N-linked initial glycan, wherein the glycosynthase comprises SEQ ID NO. 1 or SEQ ID NO. 2, and the glycosynthase comprises a mutation located within residues 176-186, residues 225-237, residues 273-289 in the sequence of SEQ ID NO. 1 or within residues 178-188, residues 227-239, residues 275-291 in the sequence of SEQ ID NO. 2, wherein the modified glycan comprises a substrate moiety and a first reactive moiety, wherein the substrate moiety is configured to react with the glycosynthase, and wherein the biomolecule comprises an antibody or antigen binding fragment thereof, a protein, or a peptide.
Embodiment 2: The method of embodiment 1, wherein the biomolecule comprises the antibody or antigen binding fragment thereof, and the N-linked initial glycan is located at a constant region of the antibody or antigen-binding fragment.
Embodiment 3: The method of embodiment 1 or embodiment 2, wherein the biomolecule comprises the antibody or antigen binding fragment thereof, and the N-linked initial glycan is located at a Fc region of the antibody or antigen-binding fragment.
Embodiment 4: The method of embodiment 3, wherein the N-linked initial glycan is located at N297 site of the Fc region.
Embodiment 5: The method of any one of embodiments 1 to 4, wherein contacting the biomolecule with the glycosynthase and the modified glycan comprises coupling the modified glycan with the N-linked initial glycan.
Embodiment 6: The method of any one of embodiments 1 to 5, wherein the substrate moiety of the modified glycan is an oxazoline moiety.
Embodiment 7: The method of any one of embodiments 1 to 6, wherein the first reactive moiety is configured to react with unsaturated moiety in a biorthogonal reaction.
Embodiment 8: The method of embodiment 7, wherein the biorthogonal reaction is a copper-free click chemistry.
Embodiment 9: The method of any one of embodiments 1 to 8, wherein the first reactive moiety comprises an azido group.
Embodiment 10: The method of any one of embodiments 1 to 9, wherein the modified glycan is a PEGylated glycan modified with a first polyethylene glycol (PEG) moiety.
Embodiment 11: The method of embodiment 10, wherein the first polyethylene glycol (PEG) moiety comprises from 2 to 72 OCH₂CH₂ subunits.
Embodiment 12: The method of embodiment 10 or embodiment 11, wherein the first PEG moiety is a linear PEG, a branched PEG, or a star PEG.
Embodiment 13: The method of any one of embodiments 10 to 12, wherein a first end of the PEGylated glycan is covalently coupled with the first reactive moiety.
Embodiment 14: The method of embodiment 13, wherein the first reactive moiety is covalently coupled with the PEG moiety.
Embodiment 15: The method of any one of embodiments 10 to 14, wherein a second end of the PEGylated glycan is covalently coupled with the substrate moiety.
Embodiment 16: The method of embodiment 15, wherein the substrate moiety is covalently coupled with a glycol moiety of the PEGylated glycan.
Embodiment 17: The method of any one of embodiments 1 to 16, wherein the modified glycan is a glycan oxazoline.
Embodiment 18: The method of embodiment 17, wherein the glycan oxazoline comprises a formula of:

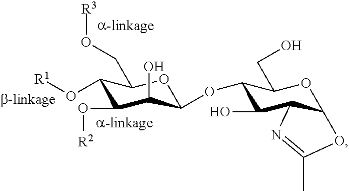

wherein R¹ is —H or N-acetyl glucosamine attached via a β-1,4 linkage, and R² and R³ are same or different and are independently selected from the group consisting of:

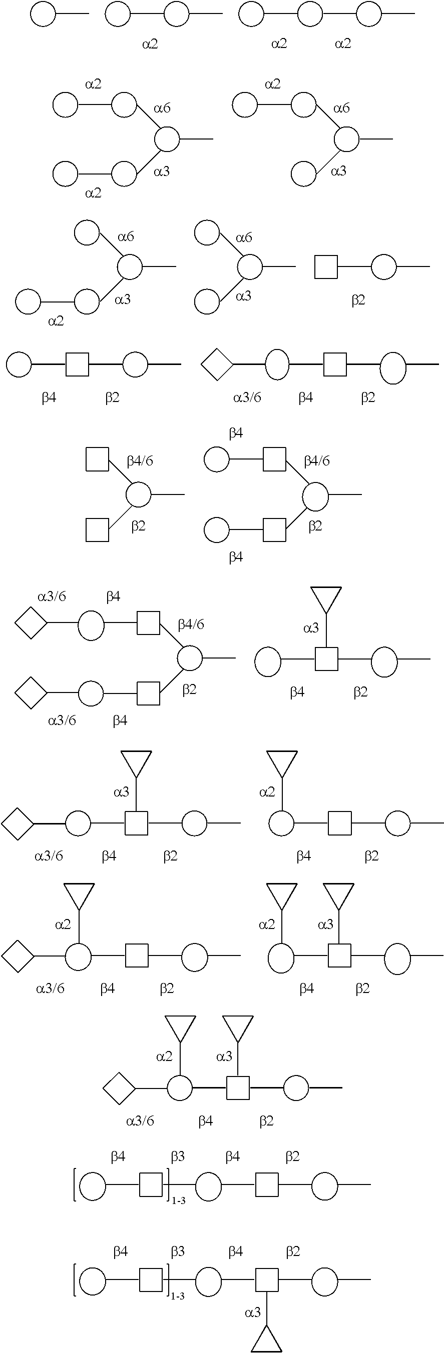

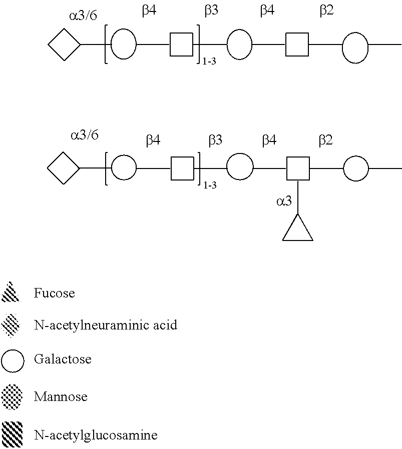

Embodiment 19: The method of any one of embodiments 1 to 18, wherein contacting the biomolecule with the glycosynthase and the modified glycan comprises: removing the N-linked initial glycan of the biomolecule thereby obtaining a deglycosylated biomolecule; and contacting the deglycosylated biomolecule with the glycosynthase in presence of the modified glycan.
Embodiment 20: The method of embodiment 19, wherein removing the N-linked initial glycan of the biomolecule thereby obtaining a deglycosylated biomolecule comprises, in absence of the modified glycan, mixing the glycosynthase and the biomolecule at a ratio of from 1:500 to 1:1, from 1:500 to 1:10, from 1:500 to 1:20, from 1:500 to 1:30, from 1:500 to 1:50, from 1:100 to 1:1, from 1:100 to 1:10, from 1:100 to 1:20, from 1:100 to 1:30, from 1:100 to 1:50, from 1:50 to 1:1, from 1:50 to 1:10, from 1:50 to 1:20, or from 1:50 to 1:30.
Embodiment 21: The method of embodiment 19 or embodiment 20, wherein the deglycosylated biomolecule comprises a GlcNAc monosaccharide.
Embodiment 22: The method of embodiment 21, wherein the GlcNAc monosaccharide is fucosylated.
Embodiment 23: The method of embodiment 21, wherein the GlcNAc monosaccharide is non-fucosylated.
Embodiment 24: The method of any one of embodiments 1 to 23, wherein contacting the biomolecule with the glycosynthase and the modified glycan comprising contact a plurality of the biomolecules with the glycosynthase and the modified glycan thereby obtaining a plurality of the first engineered bioconjugates; wherein a homogeneity of the plurality of the first engineered bioconjugate is at least or above 80%, 85%, 90%, 95%, or 99%.
Embodiment 25: The method of any one of embodiments 1 to 24, further comprising contacting the first engineered bioconjugate with a payload conjugate or a salt thereof thereby obtaining a second bioconjugate, wherein the payload conjugate comprises a formula:

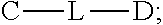

wherein C is a second reactive moiety, configured to react with the first reactive moiety of the modified glycan in a biorthogonal reaction; wherein L is a linker unit comprising a hydrophilic moiety; and wherein D is a payload.
Embodiment 26: The method of embodiment 25, wherein the second reactive moiety comprises an unsaturated moiety (e.g., an alkene moiety or an alkyne moiety).
Embodiment 27: The method of embodiment 25 or embodiment 26, wherein the second reactive moiety is a non-native and nonperturbing chemical group.
Embodiment 28: The method of any one of embodiments 25 to 27, wherein the second reactive moiety is a dibenzocyclooctyne group (DBCO), a bicyclononyne (BCN), a cyclic alkyne, a maleimide group, a α,β-unsaturated carbonyl group, or a sulfonyl pyrimidine.
Embodiment 29: The method of any one of embodiments 25 to 28, wherein the hydrophilic moiety comprises a second polyethylene glycol (PEG) moiety.
Embodiment 30: The method of embodiment 29, wherein the second PEG moiety comprises from 2 to 72 OCH₂CH₂ subunits.
Embodiment 31: The method of embodiment 29 or embodiment 30, wherein the second PEG moiety is a linear PEG, a branched PEG, or a star PEG.
Embodiment 32: The method of any one of embodiments 25 to 31, wherein the linker unit further comprises a cleavable moiety.
Embodiment 33: The method of embodiment 32, wherein the cleavable moiety is a protease sensitive peptide or a glycosidase sensitive sugar unit.
Embodiment 34: The method of any one of embodiments 25 to 33, wherein the linker unit further comprises a spacer comprising an aromatic group or amino methylene.
Embodiment 35: The method of any one of embodiments 25 to 34, wherein the linker unit comprises a formula of:

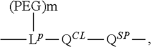

wherein (PEG)m is a PEG moiety, and m is an integer selected from 2 to 72, Q^{SP} is a spacer comprising an aromatic group or amino methylene, Q^{CL} is a cleavable moiety and is configured to link to the payload, L^{P} is a connector unit configured to link to the second reactive moiety.
Embodiment 36: The method of embodiment 35, wherein the PEG moiety comprises a formula of

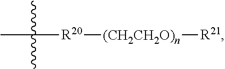

wherein the wavy line indicates the site of covalent attachment to L^{P}, wherein R²⁰ is a PEG attachment unit, wherein the PEG attachment unit is —C(O)—, —O—, —S—, —NH—, —C(O)O—, alkyl-C(O)—NH—, alkyl-NH—C(O)—, alkyl-CO₂—, alkyl-S—, or

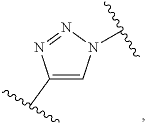

wherein R²¹ is a PEG capping unit; wherein the PEG capping unit is select from H, SO₃H, PO₃H₂, a sugar derivative, C₁-C₁₀ (hetero) alkyl group, C₃-C₁₀ (hetero) cycloalkyl group, C₂-C₁₀ alkyl-NH₂, C₁-C₁₀ alkyl-COOH, C₂-C₁₀ alkyl-NH(C₁-C₃ alkyl), C₂-C₁₀ alkyl-N (C₁-C₃ alkyl)₂, and n is selected from 8 to 72.
Embodiment 37: The method of any one of embodiments 25 to 36, wherein the linker unit has a structure of:

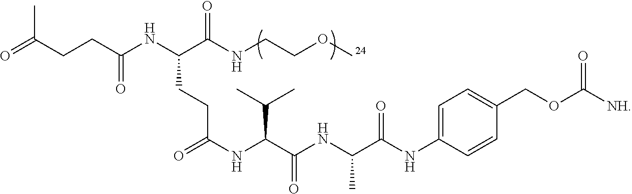

Embodiment 38: The method of any one of embodiments 25 to 37, wherein the payload is a therapeutic agent.
Embodiment 39: The method of embodiment 38, wherein the therapeutic agent comprises an anti-viral agent, an anti-bacterial agent, an immunoregulatory, an immunostimulatory agent, an anti-tumor agent, or a combination thereof.
Embodiment 40: The method of any one of embodiments 25 to 39, wherein the payload comprises a toxin, a cytokine, a growth factor, a radionuclide, a hormone, or a combination thereof.
Embodiment 41: The method of any one of embodiments 25 to 40, wherein the payload is selected from pyrrolobenzodiazepine (e.g. PBD), auristatin (e.g. MMAE, MMAF), maytansinoid (e.g. maytansine, DM1, DM4, DM21), duocarmycin, nicotinamide phosphoribosyltransferase (NAMPT) inhibitor, tubulysin, enediyne (e.g. calicheamicin), anthracycline derivative (PNU) (e.g. doxorubicin), pyrrole-based kinesin spindle protein (KSP) inhibitor, cryptophycin, drug efflux pump inhibitor, sandramycin, amanitin (e.g. alpha-amanitin), and camptothecin (e.g. exatecan, deruxtecan).
Embodiment 42: The method of any one of embodiments 25 to 41, wherein the payload conjugate comprises a structure of:

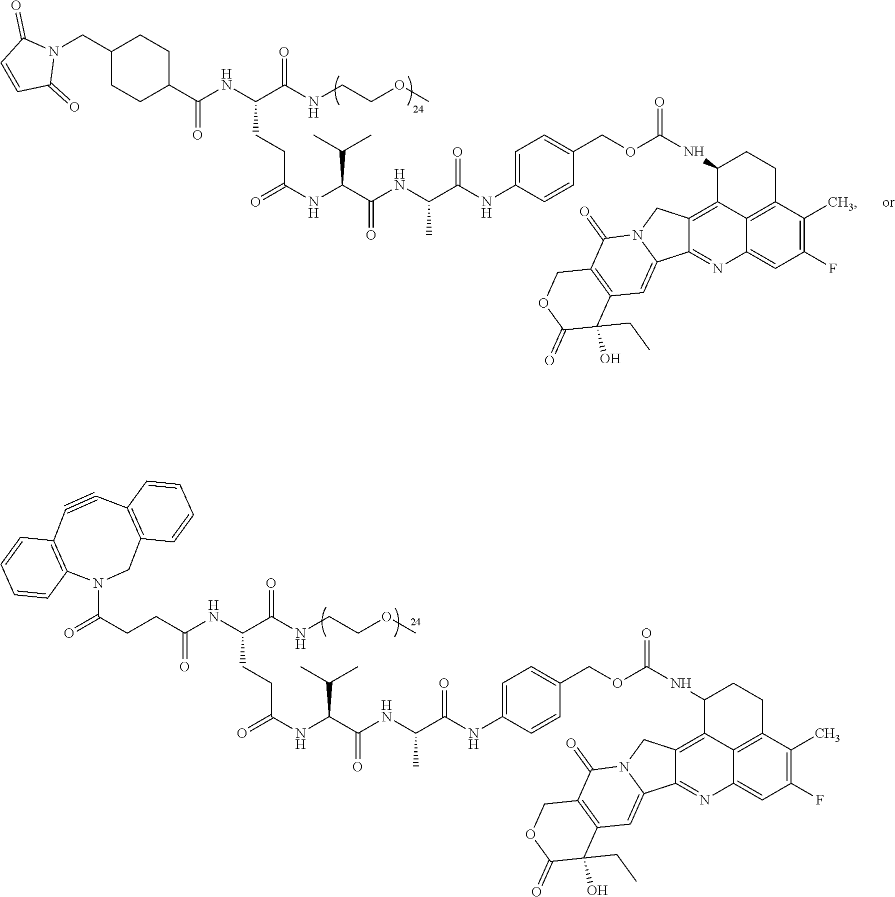

Embodiment 43: The method of any one of embodiments 1 to 42, wherein the mutation comprises an alternation at residue 183, 232, 234, 280, 281, or 282 of SEQ ID NO. 1 or residue 181, 230, 232, 278, 279, or 280 of SEQ IN NO. 2.
Embodiment 44: The method of embodiment 43, wherein the mutation comprises, in SEQ ID NO. 1, D234E, D234R, D234H, D234M, D234V, D234L, D234F, D234T, D234Q, T183Q, D232Q, D280Q, S281Q, T282Q, or in SEQ ID NO. 2, D232E, D232R, D232H, D232M, D232V, D232L, D232F, D232T, D232Q, T181Q, D230Q, D278Q, S279Q, T280Q.
Embodiment 45: The method of any one of embodiments 1 to 44, wherein the biomolecule is an anti-Globo series antigen antibody or antigen-binding fragment thereof, an anti-HER2 antibody or antigen-binding fragment thereof, an anti-CD20 antibody or antigen-binding fragment thereof, an anti-TNF-alpha antibody or antigen-binding fragment thereof, an anti-PD-1 antibody or antigen-binding fragment thereof, an anti-PD-L1 antibody or antigen-binding fragment thereof, an anti-TROP2 antibody or antigen-binding fragment thereof, an anti-EGFR antibody or antigen-binding fragment thereof, an anti-Nectin-4 antibody or antigen-binding fragment thereof, an anti-HER3 antibody or antigen-binding fragment thereof, an anti-cMet antibody or antigen-binding fragment thereof, an anti-B7H3 antibody or antigen-binding fragment thereof, an anti-B7H4 antibody or antigen-binding fragment thereof, an anti-VEGF antibody or antigen-binding fragment thereof, an anti-Claudin 18.2 antibody or antigen-binding fragment thereof, an anti-Sirp-Alpha antibody or antigen-binding fragment thereof, an TROP2×HER2 bispecific antibody, or a combination thereof.
Embodiment 46: The method of embodiment 45, wherein the Globo series antigen comprises Globo H, stage-specific embryonic antigen 4 (SSEA-4) or stage-specific embryonic antigen 3 (SSEA-3).
Embodiment 47: The method of any one of embodiments 1 to 46, wherein the biomolecule is selected from Herceptin (trastuzumab), TX05 (trastuzumab biosimilar), Perjeta (pertuzumab), Erbitux (cetuximab), Rituxan (rituximab), Vectibix (panitumumab), Humira (adalimumab), Keytruda (pembrolizumab) Bavencio (avelumab), and BSI04702 (anti-TROP2 antibody).
Embodiment 48: An engineered bioconjugate, comprising: a biomolecule; and a modified glycan, coupled with the biomolecule, wherein the modified glycan comprises (i) a first polyethylene glycol (PEG) moiety and (ii) a first reactive moiety or a resultant moiety thereof from a biorthogonal reaction; wherein the biomolecule comprises an antibody or antigen binding fragment thereof, a protein, or a peptide.
Embodiment 49: The engineered bioconjugate of embodiment 48, wherein the biomolecule comprises the antibody or antigen binding fragment thereof, and the modified glycan is coupled with the antibody or antigen-binding fragment thereof at a Fc region thereof.
Embodiment 50: The engineered bioconjugate of embodiment 49, wherein the modified glycan is coupled with the antibody or antigen-binding fragment thereof at N297 site of the Fc region.
Embodiment 51: The engineered bioconjugate of embodiment 50, wherein the modified glycan is coupled with a GlcNAc monosaccharide at the N297 site of the Fc region.
Embodiment 52: The engineered bioconjugate of embodiment 51, wherein the GlcNAc monosaccharide is fucosylated.
Embodiment 53: The engineered bioconjugate of embodiment 51, wherein the GlcNAc monosaccharide is non-fucosylated.
Embodiment 54: The engineered bioconjugate of any one of embodiments 48 to 53, wherein the first reactive moiety is configured to interact with an alkyne moiety in a biorthogonal reaction.
Embodiment 55: The engineered bioconjugate of any one of embodiments 48 to 54, wherein the biorthogonal reaction is a copper-free click chemistry.
Embodiment 56: The engineered bioconjugate of any one of embodiments 48 to 55, wherein the first reactive moiety comprises an azido group.
Embodiment 57: The engineered bioconjugate of any one of embodiments 48 to 56, wherein the first PEG moiety comprises from 2 to 72 (OCH₂CH₂) subunits.
Embodiment 58: The engineered bioconjugate of embodiment 57, wherein the first PEG moiety is a linear PEG, a branched PEG, or a star PEG.
Embodiment 59: The engineered bioconjugate of any one of embodiments 48 to 58, wherein the modified glycan is a first modified glycan, and the engineered bioconjugate further comprises a first payload moiety, wherein the first payload moiety is coupled with the first modified glycan via the resultant moiety thereof.
Embodiment 60: The engineered bioconjugate of embodiment 59, wherein the first payload moiety has a formula of:

-L-D;

wherein L is a linker unit comprising a hydrophilic moiety and is linked to the resultant moiety, and D is a payload.
Embodiment 61: The engineered bioconjugate of embodiment 59 or embodiment 60, wherein the resultant moiety comprises a triazole moiety.
Embodiment 62: The engineered bioconjugate of embodiment 61, wherein the resultant moiety comprises a DBCO-derived moiety or a maleimide-derived moiety.
Embodiment 63: The engineered bioconjugate of any one of embodiments 60 to 62, wherein the hydrophilic moiety comprises a second polyethylene glycol (PEG) moiety.
Embodiment 64: The engineered bioconjugate of embodiment 63, wherein the second PEG moiety comprises from 2 to 72 OCH₂CH₂ subunits.
Embodiment 65: The engineered bioconjugate of embodiment 63 or embodiment 64, wherein the second PEG moiety is a linear PEG, a branched PEG, or a star PEG.
Embodiment 66: The engineered bioconjugate of any one of embodiments 60 to 65, wherein the linker unit further comprises a cleavable moiety.
Embodiment 67: The engineered bioconjugate of embodiment 66, wherein the cleavable moiety is a protease sensitive peptide or a glycosidase sensitive sugar unit.
Embodiment 68: The engineered bioconjugate of any one of embodiments 60 to 67, wherein the linker unit further comprises a spacer comprising an aromatic group or amino methylene.
Embodiment 69: The engineered bioconjugate of any one of embodiments 60 to 68, wherein the linker unit comprises a formula of:

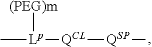

wherein (PEG)m is a PEG moiety, and m is an integer selected from 2 to 72, Q^{SP} is a spacer comprising an aromatic group or amino methylene, Q^{CL} is a cleavable moiety linked to the payload, L^{P} is a connector unit linked to the resultant moiety.
Embodiment 70: The engineered bioconjugate of embodiment 69, wherein the PEG moiety comprises a formula of

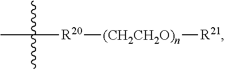

wherein the wavy line indicates the site of covalent attachment to L^{P}, wherein R²⁰ is a PEG attachment unit, wherein the PEG attachment unit is —C(O)—, —O—, —S—, —NH—, —C(O)O—, alkyl-C(O)—NH—, alkyl-NH—C(O)—, alkyl-CO₂—, alkyl-S—, or

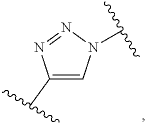

wherein R²¹ is a PEG capping unit; wherein the PEG capping unit is select from H, SO₃H, PO₃H₂, a sugar derivative, C₁-C₁₀ (hetero) alkyl group, C₃-C₁₀ (hetero) cycloalkyl group, C₂-C₁₀ alkyl-NH₂, C₁-C₁₀ alkyl-COOH, C₂-C₁₀ alkyl-NH(C₁-C₃ alkyl), C₂-C₁₀ alkyl-N (C₁-C₃ alkyl)₂, and n is selected from 8 to 72.
Embodiment 71: The engineered bioconjugate of any one of embodiments 60 to 70, wherein the linker unit has a structure of:

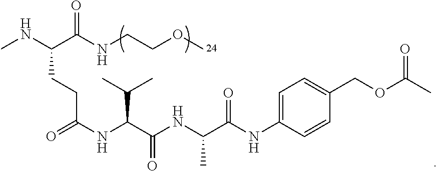

Embodiment 72: The engineered bioconjugate of any one of embodiments 59 to 71, wherein the payload is a therapeutic agent.
Embodiment 73: The engineered bioconjugate of embodiment 72, wherein the therapeutic agent comprises an anti-viral agent, an anti-bacterial agent, an immunoregulatory, an immunostimulatory agent, an anti-tumor agent, a chemotherapeutic agent, or a combination thereof.
Embodiment 74: The engineered bioconjugate of any one of embodiments 59 to 73, wherein the payload comprises a toxin, a cytokine, a growth factor, a radionuclide, a hormone, or a combination thereof.
Embodiment 75: The engineered bioconjugate of any one of embodiments 59 to 74, wherein the payload is selected from pyrrolobenzodiazepine (e.g. PBD), auristatin (e.g. MMAE, MMAF), maytansinoid (e.g. maytansine, DM1, DM4, DM21), duocarmycin, nicotinamide phosphoribosyltransferase (NAMPT) inhibitor, tubulysin, enediyne (e.g. calicheamicin), anthracycline derivative (PNU) (e.g. doxorubicin), pyrrole-based kinesin spindle protein (KSP) inhibitor, cryptophycin, drug efflux pump inhibitor, sandramycin, amanitin (e.g. alpha-amanitin), and camptothecin (e.g. exatecan, deruxtecan).
Embodiment 76: The engineered bioconjugate of any one of embodiments 59 to 75, further comprising a second payload moiety, wherein the second payload is coupled with the engineered bioconjugate via a second modified glycan.
Embodiment 77: The engineered bioconjugate of embodiment 76, wherein the first payload moiety and the second payload moiety are different.
Embodiment 78: The engineered bioconjugate of embodiment 76 or embodiment 77, wherein the first payload moiety comprises a first payload (D1) and the second payload moiety comprises a second payload (D2), and the first payload (D1) is different from the second payload (D2).
Embodiment 79: The engineered bioconjugate of any one of embodiments 76 to 78, wherein the first modified glycan is different from the second modified glycan.
Embodiment 80: The engineered bioconjugate of any one of embodiments 48 to 79, wherein the biomolecule is an anti-Globo series antigen antibody or antigen-binding fragment thereof, an anti-HER2 antibody or antigen-binding fragment thereof, an anti-CD20 antibody or antigen-binding fragment thereof, an anti-TNF-alpha antibody or antigen-binding fragment thereof, an anti-PD-1 antibody or antigen-binding fragment thereof, an anti-PD-L1 antibody or antigen-binding fragment thereof, an anti-TROP2 antibody or antigen-binding fragment thereof, an anti-EGFR antibody or antigen-binding fragment thereof, an anti-Nectin-4 antibody or antigen-binding fragment thereof, an anti-HER3 antibody or antigen-binding fragment thereof, an anti-cMet antibody or antigen-binding fragment thereof, an anti-B7H3 antibody or antigen-binding fragment thereof, an anti-B7H4 antibody or antigen-binding fragment thereof, an anti-VEGF antibody or antigen-binding fragment thereof, an anti-Claudin 18.2 antibody or antigen-binding fragment thereof, an anti-Sirp-Alpha antibody or antigen-binding fragment thereof, an TROP2×HER2 bispecific antibody, or a combination thereof.
Embodiment 81: The engineered bioconjugate of embodiment 80, wherein the Globo series antigen comprises Globo H, stage-specific embryonic antigen 4 (SSEA-4) or stage-specific embryonic antigen 3 (SSEA-3).
Embodiment 82: The engineered bioconjugate of any one of embodiments 48 to 81, wherein the biomolecule is selected from Herceptin (trastuzumab), TX05 (trastuzumab biosimilar), Perjeta (pertuzumab), Erbitux (cetuximab), Rituxan (rituximab), Vectibix (panitumumab), Humira (adalimumab), Keytruda (pembrolizumab) Bavencio (avelumab), and BSI04702 (anti-TROP2 antibody).
Embodiment 83: The engineered bioconjugate of any one of embodiments 48 to 82, prepared by the method of any one of embodiments 1 to 46.
Embodiment 84: A plurality of engineered bioconjugates, each is of the engineered bioconjugate of any one of embodiments 48 to 83, wherein a homogeneity of the plurality of engineered bioconjugates is at least or above 80%, 85%, 90%, 95%, or 99%.
Embodiment 85: A pharmaceutical composition, comprising the plurality of engineered bioconjugates of embodiment 84 and a pharmaceutically acceptable carrier.
Embodiment 86: A method for treating cancer, comprising administering to a patient in need thereof an effective amount of the pharmaceutical composition according embodiment 85.
Embodiment 87: The method of embodiment 86, wherein the cancer is a Globo series antigen, HER2, TROP2, Nectin-4, HER3, cMet, B7H3, B7H4, VEGF, Claudin 18.2, or Sirp-Alpha expressing cancer.
Embodiment 88: The method of embodiment 86 or embodiment 87, wherein the cancer is selected from the group consisting of sarcoma, skin cancer, leukemia, lymphoma, brain cancer, glioblastoma, lung cancer, breast cancer, oral cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testicular cancer, buccal cancer, oropharyngeal cancer, laryngeal cancer, prostate cancer, thyroid cancer and oral cancer.
Embodiment 89: A therapeutic conjugate comprising a formula of:

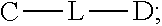

wherein C is a reactive moiety, configured to react in a biorthogonal reaction; wherein L is a linker unit comprising a hydrophilic moiety, a cleavable moiety, and a spacer; and wherein D is a therapeutic agent.
Embodiment 90: The therapeutic conjugate of embodiment 89, wherein the reactive moiety comprises an unsaturated moiety.
Embodiment 91: The therapeutic conjugate of embodiment 90, wherein the unsaturated moiety is an alkene moiety or an alkyne moiety.
Embodiment 92: The therapeutic conjugate of any one of embodiments 89 to 91, wherein the reactive moiety is a non-native and nonperturbing chemical group.
Embodiment 93: The therapeutic conjugate of any one of embodiments 89 to 92, wherein the reactive moiety is a dibenzocyclooctyne group (DBCO), a bicyclononyne (BCN), a cyclic alkyne, a maleimide group, a α,β-unsaturated carbonyl group, or a sulfonyl pyrimidine.
Embodiment 94: The therapeutic conjugate of any one of embodiments 89 to 93, wherein the hydrophilic moiety comprises a polyethylene glycol (PEG) moiety.
Embodiment 95: The therapeutic conjugate of embodiment 94, wherein the PEG moiety comprises from 2 to 72 OCH₂CH₂ subunits.
Embodiment 96: The therapeutic conjugate of embodiment 94 or embodiment 95, wherein the first PEG moiety is a linear PEG, a branched PEG, or a star PEG.
Embodiment 97: The therapeutic conjugate of any one of embodiments 89 to 96, wherein the cleavable moiety is a protease sensitive peptide, including Val-Cit, Val-Ala, Phe-Lys, Glu-Val-Cit, Glu-Val-Ala, Glu-Gly-Cit, Glu-Gly-Ala, Gly-Gly-Phe-Gly, Gly-Gly-Val-Cit, Gly-Gly-Val-Ala, or a glycosidase sensitive sugar unit, including glucuronic acid, Iduronic acid, or galactose.
Embodiment 98: The therapeutic conjugate of any one of embodiments 89 to 97, wherein the spacer comprises an aromatic group, including a 1,4-phenyl group, a 2,5-pyridyl group, a 3,6-pyridyl group, a 2,5-pyrimidyl group, 2,5-thienyl group, or amino methylene (e.g., —NH—CH₂—).
Embodiment 99: The therapeutic conjugate of any one of embodiments 89 to 98, wherein the linker unit comprises a formula of:

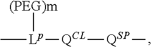

wherein (PEG)m is a PEG moiety, and m is an integer selected from 2 to 72, Q^{SP} is a spacer comprising an aromatic group or amino methylene, Q^{CL} is a cleavable moiety and is configured to link to the payload, L^{P} is a connector unit configured to link to the second reactive moiety.
Embodiment 100: The therapeutic conjugate of embodiment 99, wherein the PEG moiety comprises a formula of

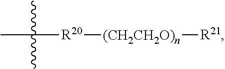

wherein the wavy line indicates the site of covalent attachment to L^{P}, wherein R²⁰ is a PEG attachment unit, wherein the PEG attachment unit is —C(O)—, —O—, —S—, —NH—, —C(O)O—, alkyl-C(O)—NH—, alkyl-NH—C(O)—, alkyl-CO₂—, alkyl-S—, or

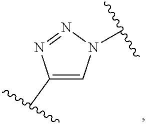

wherein R²¹ is a PEG capping unit; wherein the PEG capping unit is select from H, SO₃H, PO₃H₂, a sugar derivative, C₁-C₁₀ (hetero) alkyl group, C₃-C₁₀ (hetero) cycloalkyl group, C2-C₁₀ alkyl-NH₂, C₁-C₁₀ alkyl-COOH, C₂-C₁₀ alkyl-NH(C₁-C₃ alkyl), C₂-C₁₀ alkyl-N (C₁-C₃ alkyl)₂, and n is selected from 8 to 72.
Embodiment 101: The therapeutic conjugate of any one of embodiments 89 to 100, wherein the linker unit has a structure of:

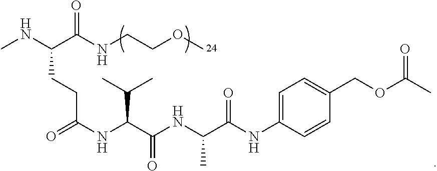

Embodiment 102: The therapeutic conjugate of any one of embodiments 89 to 111, wherein the therapeutic agent comprises a toxin, a cytokine, a growth factor, a radionuclide, a hormone, an anti-viral agent, an anti-bacterial agent, an immunoregulatory, an immunostimulatory agent, an anti-tumor agent, or a combination thereof.
Embodiment 103: The therapeutic conjugate of any one of embodiments 89 to 112, wherein the therapeutic agent is selected from pyrrolobenzodiazepine (e.g. PBD), auristatin (e.g. MMAE, MMAF), maytansinoid (e.g. maytansine, DM1, DM4, DM21), duocarmycin, nicotinamide phosphoribosyltransferase (NAMPT) inhibitor, tubulysin, enediyne (e.g. calicheamicin), anthracycline derivative (PNU) (e.g. doxorubicin), pyrrole-based kinesin spindle protein (KSP) inhibitor, cryptophycin, drug efflux pump inhibitor, sandramycin, amanitin (e.g. alpha-amanitin), and camptothecin (e.g. exatecan, deruxtecan).
Embodiment 104: The therapeutic conjugate of any one of embodiments 89 to 103, having a structure of:

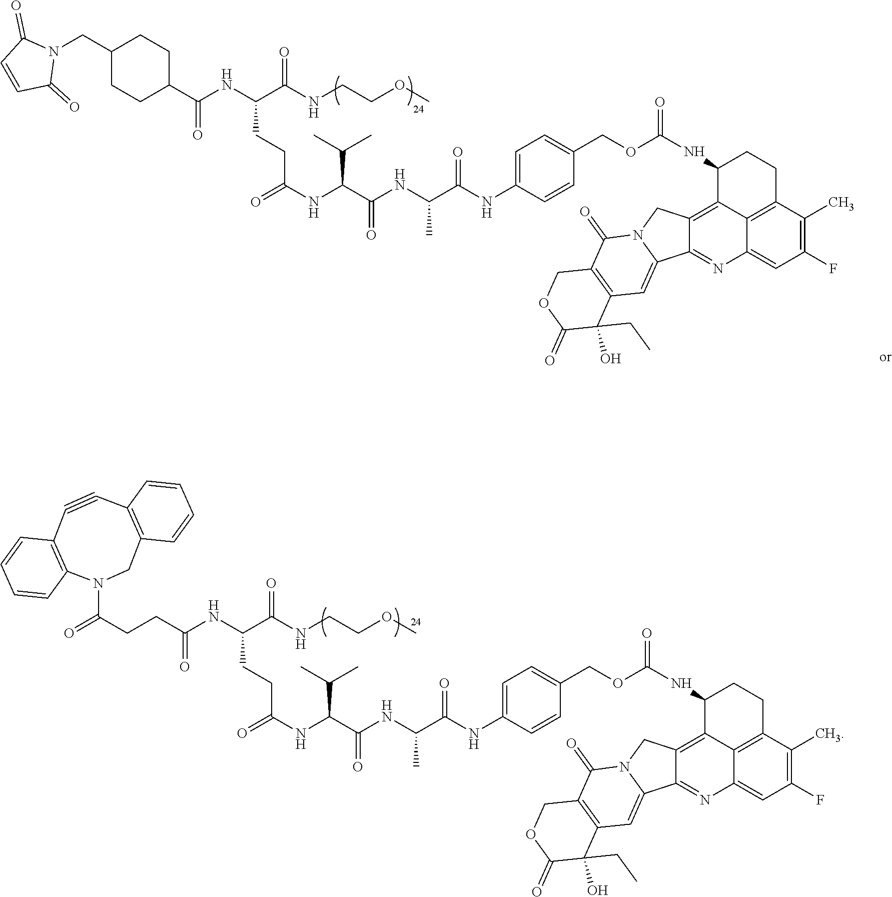

### SEQUENCE LISTING

SEQ ID NO 1:
Name: EndoSd-D232M amino acid sequence
Organism: Streptococcus dysgalactiae subsp. Dysgalactiae
MGTILGTHHDSLISVKAEEKITQVSQTSTSIDDLHYLSENSKKEFKEELSKEKVPEKVKEILSKAQQANK
QAQELAEMKVPDKIPMKPLNGPLYGGYFRTWHDKTSDPLEKDKVNSMGELPKEVDLAFVFHDWTKDYSLF
WKELATKHVPKLNKQGTRVIRTIPWRFLAGGDNSGIAEDASKYPNTPEGNKALAKAIVDEYVYKYNLDGL
DVMIEHDSIPKVNGEASDENLKRSIDVFEEIGKLIGPKGADKSRLFIMDSTYMADKNPLIERGAPYIDLL
LVQVYGSQGEQGEFQNDTKSVTKTPEERWQGYSKYIRPEQYMIGFSFYEEKAGSGNLWYDINARKDEDTA
NGINDDITGTRAERYARWQPKTGGVKGGIFSYAIDRDGVAHQPKQIAEKDKQSVKNNRPLISEITDNIFH
SNYSVSKTLKTVMLKDKAYDLIDEKDFPDKALREAVMAQVGTRKGDLERFNGTLRLDNPAIQSLEGLNKF
KKLAQLDLIGLSRIIKLDQSVLPANMKPGKDPLETVLETYKKNGKEEPAIIPPVSLTVSGLTGLKELDLS
GFDRETLAGIDAATLTSLEKVDISDNKLDLAPKTENRQIFDVMLSTVNNNAGISEQSIKFDNQKPAGNYP
QTYGATNLQLPVRQEKIDLQHQLLFGTITNQGTLINSEADYKTYRNQKIAGRNFVDPDYPYNNFKVSHDN
YTVKVTDSTLGTTTDKMLATDKEETYKVDFFSPTDKTKAVHTAKVIVGDEKTMMVNLAEGATVIKSENDE
NAQKVENGIMEYNPLSFNNKSSIIFEIKDPSLAKYWRLENDSSKDKKDYIKEAKLEVFTGQLNAEADVKT
ILEKPDNWVTVSTYSGEEKVFSHSLDNISAKYWRVTVDNKKDQYGYVSLPELQILGYPLPNADTIMKTVT
VAKELSQQKDKFPQQLLDESTAKEAVVEASLNSKLFDTGVINTNVEALKNVVDECLAYEKNKETAFKATE
DYRAAVNGVKAESVTVEEMAQLKDLIGKAAHLNSKIDAKLADREYDKDLLGLIGELTNITRTVKSFVKHH
HHHH
SEQ ID NO 2:
Name: EndoSz-D234M amino acid sequence
Organism: Streptococcus equi subsp. Zooepidemicus Sz105
MVAILAAQHDSLIRVKAEDKLVQTSPSVSAIDALHYLSENSKKEFKEELSKVEKAQPEKLKEIVSKAQQA
DKQAKTLAEMKVPEKIPMKPLKGPLYGGYFRTWHDKTSDPAEKDKVNSMGELPKEVDLAFVFHDWTKDYS
LFWQELATKHVPTLNKQGTRVIRTIPWRFLAGGDHSGIAEDAQKYPNTPEGNKALAKAIVDEYVYKYNLD
GLDVMIERDSIPKVNKEESKEGIERSIQVFEEIGKLIGPKGADKSRLFIMDSTYMADKNPLIERGAPYID
LLLVQVYGTQGEKGGFDNANHKAVDTMEERWESYSKYIRPEQYMVGFSFYEEKANSGNLWYDVNVEDDTN
PNIGSEIKGTRAERYAKWQPKTGGVKGGIFSYGIDRDGVAHPKKNGPKTPDLDKIVKSDYKVSKALKKVM
ENDKSYELIDQKDFPDKALREAVIAQVGSRRGNLERFNGTLRLDNPDIKSLEGLNKLKKLAKLELIGLSQ
ITKLDSSVLPENIKPTKDTLVSVLETYKNDDRKEEAKAIPQVALTISGLIGLKELNLAGFDRDSLAGIDA
ASLTSLEKVDLSSNKLDLAAGTENRQILDTMLATVTKHGGVSEKTFVFDHQKPTGLYPDTYGTKSLQLPV
ANDTIDLQAKLLFGTVTNQGTLINSEADYKAYQEQEIAGHRFVDSSYDYKAFAVTYKDYKIKVTDSTLGV
TDHKDLSTSKEETYKVEFFSPINSTKPVHEAKIVVGEEKTMMVNLAEGATIIGGDADPTNAKKVFDGLLN
NDTTTLSTSNKASIIFELKEPGLVKHWRFENDSKISKADYIKEAKLEAFVGHLEDSSKVKDSLEKSTEWV
TVSDYSGEAQEFSQPLNNIGAKYWRITIDNKKSQYGYVSLPELQIIGHRLPEAATVMTTMAAAEELSQQK
DKFSQEQLKELEVKVAALKAALDNKMFNADTINASFADVKAYIDKLLADAAGKKTLGKATKEAQPVATDA
KEKAESENPKADHHHHHH
SEQ ID NO 3:
Name: EndoSd-D232M nucleic acid sequence
Organism: Streptococcus dysgalactiae subsp. Dysgalactiae
ATGGGCACCATCCTGGGTACCCACCACGACAGCCTGATCAGCGTGAAGGCGGAGGAAAAAATTACCCAAG
TTAGCCAAACCAGCACCAGCATTGACGATCTGCACTACCTGAGCGAAAACAGCAAGAAAGAGTTCAAAGA
GGAGCTGAGCAAGGAGAAAGTGCCGGAAAAGGTTAAAGAGATCCTGAGCAAAGCGCAGCAAGCGAACAAG
CAGGCGCAAGAGCTGGCGGAAATGAAGGTGCCGGACAAAATTCCGATGAAGCCGCTGAACGGTCCGCTGT
ATGGTGGCTACTTTCGTACCTGGCACGACAAAACCAGCGATCCGCTGGAAAAGGACAAAGTTAACAGCAT
GGGCGAACTGCCGAAAGAGGTGGATCTGGCGTTCGTTTTTCACGACTGGACCAAAGATTATAGCCTGTTC
TGGAAAGAGCTGGCGACCAAGCACGTGCCGAAGCTGAACAAACAGGGTACCCGTGTTATCCGTACCATTC
CGTGGCGTTTTCTGGCGGGTGGCGACAACAGCGGTATTGCGGAAGATGCGAGCAAGTACCCGAACACCCC
GGAGGGTAACAAAGCGCTGGCGAAGGCGATTGTGGACGAATACGTTTATAAATACAACCTGGACGGTCTG
GATGTGATGATCGAGCACGATAGCATTCCGAAAGTTAACGGCGAAGCGAGCGACGAGAACCTGAAGCGTA
GCATCGATGTGTTCGAGGAAATCGGTAAACTGATTGGTCCGAAAGGCGCGGACAAGAGCCGTCTGTTTAT
TATGGACAGCACCTATATGGCGGATAAGAACCCGCTGATCGAACGTGGCGCGCCGTATATTGACCTGCTG
CTGGTGCAGGTTTACGGTAGCCAGGGCGAGCAGGGTGAATTCCAAAACGATACCAAAAGCGTTACCAAGA
CCCCGGAGGAACGTTGGCAGGGCTATAGCAAATACATCCGTCCGGAGCAATATATGATTGGTTTCAGCTT
TTACGAGGAAAAGGCGGGTAGCGGCAACCTGTGGTACGACATCAACGCGCGTAAAGACGAAGATACCGCG
AACGGCATCAACGACGATATTACCGGTACCCGTGCGGAGCGTTATGCGCGTTGGCAGCCGAAAACCGGTG
GCGTGAAGGGTGGCATCTTTAGCTACGCGATTGACCGTGATGGTGTTGCGCACCAGCCGAAGCAAATCGC
GGAAAAGGACAAACAAAGCGTGAAAAACAACCGTCCGCTGATCAGCGAGATTACCGATAACATTTTCCAC
AGCAACTATAGCGTGAGCAAGACCCTGAAAACCGTTATGCTGAAGGACAAAGCGTACGACCTGATCGATG
AAAAAGACTTTCCGGATAAAGCGCTGCGTGAGGCGGTGATGGCGCAGGTTGGCACCCGTAAGGGTGACCT
GGAACGTTTCAACGGCACCCTGCGTCTGGATAACCCGGCGATCCAGAGCCTGGAGGGTCTGAACAAGTTT
AAGAAACTGGCGCAACTGGACCTGATTGGCCTGAGCCGTATCATTAAACTGGATCAAAGCGTGCTGCCGG
CGAACATGAAGCCGGGTAAAGACCCGCTGGAAACCGTTCTGGAGACCTACAAGAAAAACGGCAAAGAGGA
GCCGGCGATCATTCCGCCGGTTAGCCTGACCGTTAGCGGTCTGACCGGTCTGAAAGAACTGGACCTGAGC
GGCTTCGATCGTGAGACCCTGGCGGGTATCGATGCGGCGACCCTGACCAGCCTGGAAAAGGTGGACATTA
GCGATAACAAACTGGACCTGGCGCCGAAGACCGAGAACCGTCAGATCTTCGATGTGATGCTGAGCACCGT
TAACAACAACGCGGGTATCAGCGAGCAGAGCATTAAATTTGACAACCAAAAGCCGGCGGGCAACTATCCG
CAAACCTACGGTGCGACCAACCTGCAGCTGCCGGTTCGTCAAGAAAAAATCGACCTGCAGCACCAACTGC
TGTTCGGCACCATCACCAACCAGGGTACCCTGATTAACAGCGAGGCGGATTATAAAACCTACCGTAACCA
AAAGATTGCGGGTCGTAACTTCGTGGACCCGGATTATCCGTACAACAACTTTAAAGTTAGCCACGACAAC
TATACCGTGAAGGTTACCGATAGCACCCTGGGCACCACCACCGACAAAATGCTGGCGACCGATAAAGAGG
AAACCTACAAGGTGGACTTCTTTAGCCCGACCGATAAGACCAAAGCGGTTCACACCGCGAAAGTGATCGT
TGGCGACGAAAAGACCATGATGGTGAACCTGGCGGAGGGTGCGACCGTTATCAAAAGCGAGAACGATGAA
AACGCGCAGAAGGTTTTCAACGGTATTATGGAATATAACCCGCTGAGCTTCAACAACAAGAGCAGCATCA
TTTTTGAGATCAAAGACCCGAGCCTGGCGAAGTATTGGCGTCTGTTCAACGATAGCAGCAAAGACAAGAA
AGATTACATCAAGGAAGCGAAACTGGAAGTGTTTACCGGTCAGCTGAACGCGGAAGCGGACGTTAAAACC
ATTCTGGAGAAGCCGGATAACTGGGTGACCGTTAGCACCTATAGCGGCGAGGAAAAGGTGTTTAGCCACA
GCCTGGACAACATCAGCGCGAAATACTGGCGTGTGACCGTTGACAACAAGAAAGATCAGTATGGCTACGT
TAGCCTGCCGGAGCTGCAAATCCTGGGTTACCCGCTGCCGAACGCGGATACCATTATGAAAACCGTGACC
GTTGCGAAGGAACTGAGCCAGCAAAAGGACAAATTCCCGCAGCAACTGCTGGATGAGAGCACCGCGAAGG
AAGCGGTGGTTGAGGCGAGCCTGAACAGCAAACTGTTTGACACCGGTGTGATCAACACCAACGTTGAAGC
GCTGAAGAACGTGGTTGATGAGTGCCTGGCGTATGAAAAGAACAAAGAGACCGCGTTCAAGGCGACCGAA
GACTACCGTGCGGCGGTGAACGGTGTTAAAGCGGAGAGCGTGACCGTTGAGGAAATGGCGCAGCTGAAAG
ATCTGATCGGCAAGGCGGCGCACCTGAACAGCAAAATTGACGCGAAGCTGGCGGATCGTGAATACGACAA
AGATCTGCTGGGCCTGATCGGCGAGCTGACCAACATTACCCGTACCGTGAAAAGCTTTGTTAAGTGA
SEQ ID NO 4:
Name: EndoSz-D234M nucleic acid sequence
Organism: Streptococcus equi subsp. Zooepidemicus Sz105
ATGGTTGCGATCCTGGCGGCGCAACACGATAGCCTGATTCGTGTGAAGGCGGAGGACAAACTGGTGCAGA
CCAGCCCGAGCGTTAGCGCGATTGATGCGCTGCACTACCTGAGCGAAAACAGCAAGAAAGAATTCAAAGA
GGAACTGAGCAAGGTTGAAAAAGCGCAACCGGAGAAGCTGAAAGAAATCGTGAGCAAGGCGCAGCAAGCG
GACAAGCAGGCGAAAACCCTGGCGGAGATGAAGGTTCCGGAAAAAATTCCGATGAAGCCGCTGAAAGGCC
CGCTGTATGGTGGCTACTTTCGTACCTGGCACGATAAAACCAGCGACCCGGCGGAGAAGGATAAAGTGAA
CAGCATGGGCGAGCTGCCGAAAGAAGTGGACCTGGCGTTCGTTTTTCACGATTGGACCAAGGACTATAGC
CTGTTCTGGCAAGAACTGGCGACCAAACACGTTCCGACCCTGAACAAGCAGGGCACCCGTGTGATCCGTA
CCATTCCGTGGCGTTTTCTGGCGGGTGGCGATCACAGCGGTATCGCGGAGGACGCGCAGAAATACCCGAA
CACCCCGGAAGGCAACAAGGCGCTGGCGAAAGCGATTGTGGATGAGTACGTTTATAAGTACAACCTGGAC
GGTCTGGATGTTATGATCGAACGTGACAGCATTCCGAAGGTGAACAAAGAGGAAAGCAAAGAGGGTATCG
AACGTAGCATTCAGGTTTTCGAGGAAATCGGCAAGCTGATTGGTCCGAAGGGCGCGGATAAAAGCCGTCT
GTTTATCATGGATAGCACCTATATGGCGGACAAGAACCCGCTGATCGAGCGTGGTGCGCCGTATATTGAC
CTGCTGCTGGTGCAGGTTTACGGTACCCAGGGCGAAAAAGGTGGCTTCGATAACGCGAACCACAAGGCGG
TTGACACCATGGAGGAACGTTGGGAGAGCTATAGCAAATACATCCGTCCGGAACAATATATGGTGGGTTT
CAGCTTTTACGAGGAAAAGGCGAACAGCGGCAACCTGTGGTACGACGTGAACGTTGAGGACGATACCAAC
CCGAACATCGGTAGCGAGATTAAAGGCACCCGTGCGGAACGTTATGCGAAGTGGCAGCCGAAAACCGGTG
GCGTTAAGGGTGGCATCTTTAGCTACGGTATTGACCGTGATGGCGTGGCGCACCCGAAGAAAAACGGTCC
GAAAACCCCGGACCTGGATAAGATCGTGAAAAGCGATTATAAAGTTAGCAAAGCGCTGAAGAAAGTTATG
GAGAACGACAAGAGCTACGAACTGATCGACCAAAAGGATTTCCCGGACAAAGCGCTGCGTGAGGCGGTGA
TTGCGCAGGTTGGTAGCCGTCGTGGCAACCTGGAACGTTTTAACGGTACCCTGCGTCTGGATAACCCGGA
CATCAAAAGCCTGGAGGGCCTGAACAAACTGAAGAAACTGGCGAAGCTGGAACTGATCGGTCTGAGCCAA
ATTACCAAGCTGGATAGCAGCGTTCTGCCGGAGAACATTAAGCCGACCAAAGACACCCTGGTGAGCGTTC
TGGAAACCTACAAAAACGACGATCGTAAGGAAGAGGCGAAAGCGATCCCGCAGGTGGCGCTGACCATTAG
CGGTCTGACCGGCCTGAAGGAGCTGAACCTGGCGGGTTTCGACCGTGATAGCCTGGCGGGTATTGATGCG
GCGAGCCTGACCAGCCTGGAGAAAGTGGATCTGAGCAGCAACAAGCTGGACCTGGCGGCGGGTACCGAAA
ACCGTCAAATTCTGGACACCATGCTGGCGACCGTGACCAAACACGGTGGCGTTAGCGAGAAGACCTTCGT
GTTTGATCACCAGAAACCGACCGGTCTGTATCCGGACACCTACGGCACCAAGAGCCTGCAGCTGCCGGTT
GCGAACGATACCATCGACCTGCAAGCGAAACTGCTGTTCGGTACCGTGACCAACCAGGGCACCCTGATCA
ACAGCGAAGCGGACTATAAGGCGTACCAGGAGCAAGAAATTGCGGGCCACCGTTTCGTTGATAGCAGCTA
TGACTACAAAGCGTTTGCGGTGACCTACAAGGATTACAAGATCAAGGTTACCGACAGCACCCTGGGTGTG
ACCGATCACAAAGACCTGAGCACCAGCAAAGAGGAGACCTATAAAGTTGAGTTCTTTAGCCCGATCAACA
GCACCAAACCGGTGCACGAAGCGAAGATTGTGGTTGGCGAGGAAAAGACCATGATGGTTAACCTGGCGGA
GGGCGCGACCATCATTGGTGGCGACGCGGACCCGACCAACGCGAAGAAAGTGTTCGATGGCCTGCTGAAC
AACGACACCACCACCCTGAGCACCAGCAACAAAGCGAGCATCATTTTTGAGCTGAAAGAACCGGGTCTGG
TGAAGCACTGGCGTTTCTTTAACGATAGCAAGATCAGCAAAGCGGACTACATTAAAGAGGCGAAACTGGA
AGCGTTCGTTGGTCACCTGGAAGATAGCAGCAAGGTGAAAGACAGCCTGGAGAAAAGCACCGAATGGGTG
ACCGTTAGCGATTATAGCGGCGAGGCGCAGGAATTTAGCCAACCGCTGAACAACATCGGCGCGAAGTACT
GGCGTATCACCATTGACAACAAGAAAAGCCAGTATGGTTACGTTAGCCTGCCGGAGCTGCAAATCATTGG
TCATCGTCTGCCGGAAGCGGCGACCGTGATGACCACCATGGCGGCGGCGGAGGAACTGAGCCAGCAAAAG
GATAAATTCAGCCAGGAGCAACTGAAGGAGCTGGAAGTGAAAGTTGCGGCGCTGAAGGCGGCGCTGGATA
ACAAAATGTTCAACGCGGACACCATCAACGCGAGCTTTGCGGACGTTAAAGCGTACATTGATAAGCTGCT
GGCGGACGCGGCGGGTAAGAAAACCCTGGGCAAAGCGACCAAAGAGGCGCAGCCGGTGGCGACCGATGCG
AAGGAAAAAGCGGAGAGCGAAAACCCGAAGGCGGACTAA

What is claimed is:

1. A method for preparing an engineered bioconjugate, comprising contacting a biomolecule with a glycosynthase and a modified glycan thereby obtaining a first engineered bioconjugate,
wherein the biomolecule further comprises a N-linked initial glycan;
wherein the glycosynthase comprises SEQ ID NO. 1 or SEQ ID NO. 2, and the glycosynthase comprises a mutation located within residues 176-186, residues 225-237, residues 273-289 in the sequence of SEQ ID NO. 1 or within residues 178-188, residues 227-239, residues 275-291 in the sequence of SEQ ID NO. 2;
wherein the modified glycan comprises a substrate moiety and a first reactive moiety;
wherein the substrate moiety is configured to react with the glycosynthase;
wherein the biomolecule comprises an antibody or antigen binding fragment thereof, a protein, or a peptide; and
wherein contacting the biomolecule with the glycosynthase and the modified glycan comprises coupling the modified glycan with the N-linked initial glycan.

2. The method of claim 1, wherein the biomolecule comprises the antibody or antigen binding fragment thereof, and the N-linked initial glycan is located at a N297 site of a Fc constant region of the antibody or antigen-binding fragment.

3. (canceled)

4. (canceled)

5. (canceled)

6. The method of claim 1, wherein the first reactive moiety is configured to react with unsaturated moiety in a biorthogonal reaction, and the first reactive moiety comprises an azido group.

7. (canceled)

8. (canceled)

9. The method of claim 1, wherein the modified glycan is a PEGylated glycan modified with a first polyethylene glycol (PEG) moiety comprising from 2 to 72 OCH₂CH_{2 subunits.}

10. (canceled)

11. (canceled)

12. The method of claim 9, wherein a first end of the PEGylated glycan is covalently coupled with the first reactive moiety, and a second end of the PEGylated glycan is covalently coupled with the substrate moiety.

13. (canceled)

14. The method of claim 1, wherein the modified glycan is a glycan oxazoline, comprising a formula of: 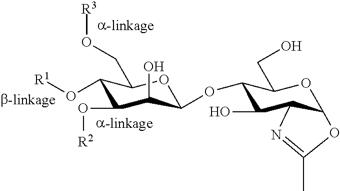
wherein R¹ is —H or N-acetyl glucosamine attached via a β-1,4 linkage, and R² and R³ are same of different and are independently selected from the group consisting of:

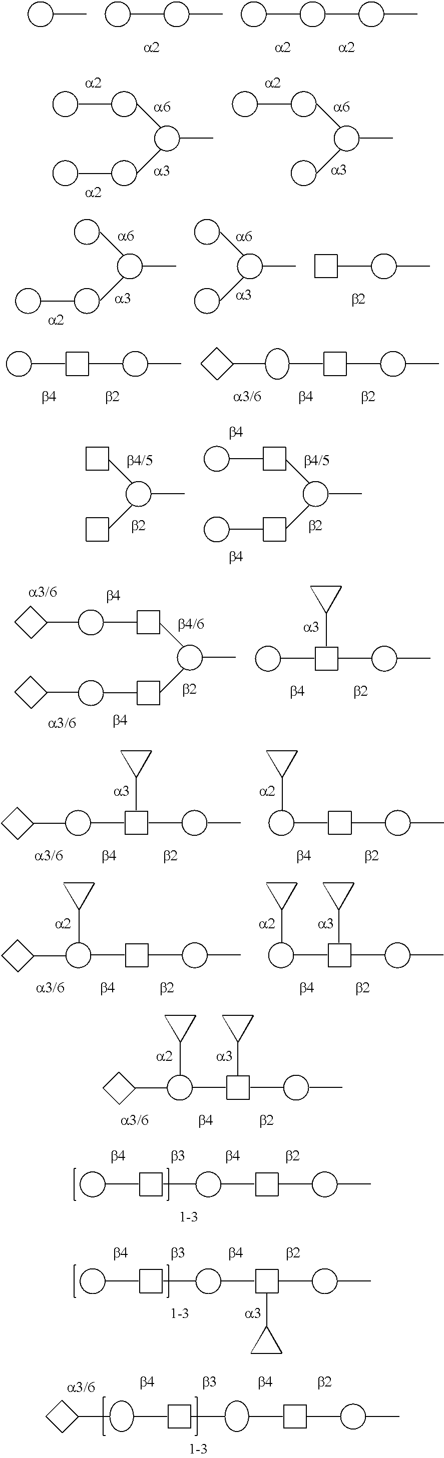

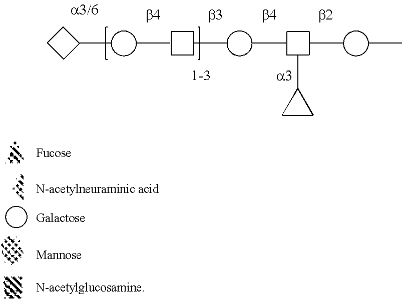

15. (canceled)

16. The method of claim 1, wherein contacting the biomolecule with the glycosynthase and the modified glycan comprises:
removing the N-linked initial glycan of the biomolecule thereby obtaining a deglycosylated biomolecule; and
contacting the deglycosylated biomolecule with the glycosynthase in presence of the modified glycan.

17. The method of claim 16, wherein removing the N-linked initial glycan of the biomolecule thereby obtaining a deglycosylated biomolecule comprises, in the absence of the modified glycan, mixing the glycosynthase and the biomolecule at a ratio of from 1:500 to 1:1.

18. The method of claim 16, wherein the deglycosylated biomolecule comprises a GlcNAc monosaccharide, which is fucosylated or non-fucosylated.

19. (canceled)

20. The method of claim 1, wherein contacting the biomolecule with the glycosynthase and the modified glycan comprising contact a plurality of the biomolecule with the glycosynthase and the modified glycan thereby obtaining a plurality of the first engineered bioconjugates; wherein a homogeneity of the plurality of the first engineered bioconjugates is above 80%.

21. The method of claim 1,
wherein the mutation comprises an alternation at residue 183, 232, 234, 280, 281, or 282 of SEQ ID NO. 1, and wherein the alteration at 183 comprises T183Q, the alteration at 232 comprises D232Q, the alteration residue 234 comprises D234E, D234R, D234H, D234M, D234V, D234L, D234F, D234T, or D234Q, the alteration at residue 280 comprises D280Q, the alteration at residue 281 comprises S281Q, or the alteration at residue 282 comprises T282Q, or
wherein the mutation comprises an alternation at residue 181, 230, 232, 278, 279, or 280 of SEQ IN NO. 2, and wherein the alteration at 181 comprises T181Q, the alteration at residue 230 comprises D230Q, the alteration at residue 232 comprises D232E, D232R, D232H, D232M, D232V, D232L, D232F, D232T, or D232Q, the alteration at residue 278 comprises D278Q, the alteration at residue 279 comprises S279Q, or the alteration at residue 280 comprises T280Q.

22. (canceled)

23. The method of claim 1, wherein the biomolecule is an anti-Globo series antigen antibody or antigen-binding fragment thereof, an anti-HER2 antibody or antigen-binding fragment thereof, an anti-CD20 antibody or antigen-binding fragment thereof, an anti-TNF-alpha antibody or antigen-binding fragment thereof, an anti-PD-1 antibody or antigen-binding fragment thereof, an anti-PD-L1 antibody or antigen-binding fragment thereof, an anti-TROP2 antibody or antigen-binding fragment thereof, an anti-EGFR antibody or antigen-binding fragment thereof, an anti-Nectin-4 antibody or antigen-binding fragment thereof, an anti-HER3 antibody or antigen-binding fragment thereof, an anti-cMet antibody or antigen-binding fragment thereof, an anti-B7H3 antibody or antigen-binding fragment thereof, an anti-B7H4 antibody or antigen-binding fragment thereof, an anti-VEGF antibody or antigen-binding fragment thereof, an anti-Claudin 18.2 antibody or antigen-binding fragment thereof, an anti-Sirp-Alpha antibody or antigen-binding fragment thereof, an TROP2×HER2 bispecific antibody, or a combination thereof.

24. (canceled)

25. The method of claim 1, wherein the biomolecule is selected from Herceptin (trastuzumab), TX05 (trastuzumab biosimilar), Perjeta (pertuzumab), Erbitux (cetuximab), Rituxan (rituximab), Vectibix (panitumumab), Humira (adalimumab), Keytruda (pembrolizumab) Bavencio (avelumab), and R4702 (anti-TROP2 antibody).

26. An engineered bioconjugate, comprising:
a biomolecule and
a modified glycan, coupled with the biomolecule;
wherein the modified glycan comprises:
(i) a first polyethylene glycol (PEG) moiety, and
(ii) a first reactive moiety or a resultant moiety thereof from a biorthogonal reaction;
wherein the resultant moiety comprises a triazole moiety, a DBCO-derived moiety, or a maleimide-derived moiety;
wherein the biomolecule comprises an antibody or antigen binding fragment thereof, a protein, or a peptide;
wherein the first reactive moiety comprises an azido group and is configured to interact with an alkyne moiety in a biorthogonal reaction; and
wherein the biorthogonal reaction is a copper-free click chemistry.

27. (canceled)

28. (canceled)

29. (canceled)

30. (canceled)

31. (canceled)

32. (canceled)

33. The engineered bioconjugate of a claim 26, further comprising a payload moiety, wherein the payload moiety is coupled with the modified glycan via the resultant moiety.

34. (canceled)

35. (canceled)

36. (canceled)

37. The engineered bioconjugate of claim 33, wherein the payload moiety is selected from pyrrolobenzodiazepine, auristatin, maytansinoid duocarmycin, nicotinamide phosphoribosyltransferase (NAMPT) inhibitor, tubulysin, enediyne, anthracycline derivative (PNU), pyrrole-based kinesin spindle protein (KSP) inhibitor, cryptophycin, drug efflux pump inhibitor, sandramycin, amanitin, and camptothecin.

38. The engineered bioconjugate of claim 26, wherein the biomolecule is an anti-Globo series antigen antibody, an anti-HER2 antibody, an anti-CD20 antibody, an anti-TNF-alpha antibody, an anti-PD-1 antibody, an anti-PD-L1 antibody, an anti-TROP2 antibody, an anti-EGFR antibody, an anti-Nectin-4 antibody, an anti-HER3 antibody, an anti-cMet antibody, an anti-B7H3 antibody, an anti-B7H4 antibody, an anti-VEGF antibody, an anti-Claudin 18.2 antibody, an anti-Sirp-Alpha antibody, an TROP2×HER2 bispecific antibody, or a combination thereof.

39. (canceled)

40. (canceled)

41. The engineered bioconjugate of claim 26 prepared by the method of claim 1.

42. A pharmaceutical composition, comprising a plurality of engineered bioconjugates and a pharmaceutically acceptable carrier, wherein each engineered bioconjugate of the plurality is the engineered bioconjugate of claim 26.

43. A method for treating cancer, comprising administering to a patient in need thereof an effective amount of the pharmaceutical composition according claim 42.

44. The method of claim 43, wherein the cancer is a Globo series antigen, HER2, TROP2, Nectin-4, HER3, cMet, B7H3, B7H4, VEGF, Claudin 18.2, Sirp-Alpha expressing cancer.

45. The method of claim 43, wherein the cancer is selected from the group consisting of sarcoma, skin cancer, leukemia, lymphoma, brain cancer, glioblastoma, lung cancer, breast cancer, oral cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testicular cancer, buccal cancer, oropharyngeal cancer, laryngeal cancer, prostate cancer, thyroid cancer and oral cancer.

46. A therapeutic conjugate comprising a formula of: 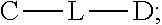
wherein C is a reactive moiety, configured to react in a biorthogonal reaction;
wherein L is a linker unit comprising a hydrophilic moiety, a cleavable moiety, and a spacer;
wherein D is a therapeutic agent; and
wherein the reactive moiety comprises an unsaturated moiety; optionally wherein the unsaturated moiety is an alkene moiety or an alkyne moiety; and/or wherein the reactive moiety is a non-native and nonperturbing chemical group; optionally wherein the reactive moiety is a dibenzocyclooctyne group (DBCO), a bicyclononyne (BCN), a cyclic alkyne, a maleimide group, a α,β-unsaturated carbonyl group, or a sulfonyl pyrimidine.

47. (canceled)

48. (canceled)

49. (canceled)

50. (canceled)

51. (canceled)

52. (canceled)

53. (canceled)

54. (canceled)

55. The therapeutic conjugate of claim 46, wherein the linker unit comprises a formula of: 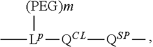
wherein (PEG)m is a polyethylene glycol moiety, and m is an integer selected from 2 to 72, Q^{SP} is a spacer comprising an aromatic group or amino methylene, Q^{CL} is a cleavable moiety and is configured to link to the payload, L^{P} is a connector unit configured to link to the second reactive moiety; or
wherein the linker unit has a structure of

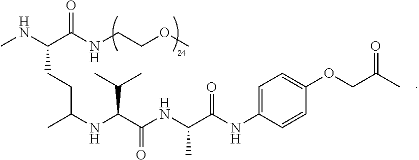

56. (canceled)

57. (canceled)

58. (canceled)

59. (canceled)

60. The therapeutic conjugate of claim 46, having a structure of: [chemical structure]
